(19) United States
(12) Patent Application Publication
(10) Pub. No.: US 2021/0198734 A1
(43) Pub. Date: Jul. 1, 2021

(54) DIRECT NUCLEIC ACID SEQUENCING METHOD

(71) Applicant: New York Institute of Technology, Old Westbury, NY (US)

(72) Inventors: Shenglong Zhang, Fort Lee, NJ (US); Ning Zhang, New York, NY (US)

(21) Appl. No.: 17/058,167

(22) PCT Filed: May 24, 2019
(86) PCT No.: PCT/US2019/033920

§ 371 Date: Nov. 24, 2020

Related U.S. Application Data
(60) Provisional application No. 62/676,703, filed on May 25, 2018.
(60) Provisional application No. 62/730,592, filed on Sep. 13, 2018.
(60) Provisional application No. 62/800,054, filed on Feb. 1, 2019.
(60) Provisional application No. 62/833,964, filed on Apr. 15, 2019.

(51) Int. Cl. C12Q 1/6872; C12N 15/10

(52) U.S. Cl. CPC C12Q 1/6872; C12N 2310/3517; C12N 15/101

(57) ABSTRACT

The present disclosure relates generally to novel methods for nucleic acid sequencing. Specifically, the invention relates to a liquid chromatography-mass-spectrometry (LC-MS) based technique for direct sequencing of RNA without cDNA. The technique allows one to simultaneously read an RNA sequence with single nucleotide resolution while determining the presence, type and location of a wide spectrum of RNA modifications.

### CROSS-REFERENCE TO RELATED APPLICATIONS

This application claims benefit and priority to U.S. Provisional Application Nos. 62/676,703, filed May 25, 2018; 62/730,592, filed Sep. 13, 2018; 62/800,054, filed Feb. 1, 2019; and 62/833,964 filed Apr. 15, 2019, which are all incorporated herein by reference in their entireties.

### TECHNICAL FIELD

The present disclosure relates generally to novel methods for nucleic acid sequencing. Specifically, the invention relates to a liquid chromatography-mass-spectrometry (LC-MS) based technique for direct sequencing of RNA without prior complementary DNA (cDNA) synthesis. The technique allows one to simultaneously read target RNA sequences with single nucleotide resolution while detecting the presence, type, location and quantity of a wide spectrum of target RNA modifications.

### BACKGROUND

Mass spectrometry (MS) is an essential tool for studying protein modifications (1), where peptide fragmentation produces “ladders” that reveal the identity and position of various amino acid modifications. As of yet, a similar approach is not yet feasible for nucleic acids, because in situ fragmentation techniques providing satisfactory sequence coverage do not exist. A number of major challenges are associated with such nucleic acids sequencing methods. One is that the process of preparing mass ladders needed for RNA sequencing also leads to the generation of other non-mass ladder fragments and mass adducts—where impurities or other molecules or their metal ions which are not related to RNA sequencing, can come along with the RNA mass ladder fragments and obscure the true masses of the ladder fragments.

Ideally, ladder cleavage should be highly uniform with one random cut on each RNA strand, without sequence preference/specificity. However, the structural/cleavage uniformity of ladder sequences generated by the prerequisite RNA degradation is often mixed with undesired fragments with multiple cuts on each RNA strand (internal fragments), complicating downstream data analysis. The presence of both internal fragments and mass adducts results in “noise” in the data that can interfere with data analysis for sequencing, because it is very challenging to single out the desired ladder fragments needed for sequencing from the entire mass data even for a single stranded RNA. Thus, methods to date do not efficiently permit the efficient sequencing of mixtures of RNA molecules such as those derived from a biological sample.

Aberrant nucleic acid modifications, especially methylations and pseudouridylations in RNA, have been correlated to the development of major diseases like breast cancer, type-2 diabetes, and obesity (2,3), each of which affects millions of people around of the world. Despite their significance, the available tools to reliably identify, locate, and quantify modifications in RNA are very limited. As a result, the function of most of such modifications remains largely unknown.

Accordingly, methods are needed to facilitate the efficient sequencing of RNA molecules, including, for example, tRNAs, siRNAs, therapeutic synthetic oligoribonucleotides having pharmacokinetic properties, mixtures of RNA molecules, as well as detection of modifications of such RNA molecules.

### SUMMARY

The current disclosure is related to a direct, liquid-chromatography-mass spectrometry (herein referred to as LC-MS) based RNA sequencing method which can be used to directly sequence RNA without the need of prior cDNA synthesis, simultaneously determine the nucleotide sequence of an RNA molecule with single nucleotide resolution, as well as, reveal the presence, type, location and quantity of RNA modifications. The disclosed method can be used to determine the type, location and quantity of each modification within the RNA sample. Such techniques can be used advantageously to correlate the biological functions of any given RNA molecule with its associated modifications and for quality control of RNA-based therapeutics.

The LC-MS-based RNA sequencing methods disclosed herein, advantageously provide methods that enable sequencing of purified RNA samples, as well as samples containing multiple RNA species, including mixtures of RNA derived from a biological sample. This strategy can be applied to the de novo sequencing of RNA sequences carrying both canonical and structurally atypical nucleosides. The methods provide a simplified means for analyzing LC-MS-based data through efficient labeling of RNA at its 3′ and/or 5′ ends, thus enabling separation of 3′ ladder and 5′ ladder RNA pools for MS-based analysis.

In an embodiment, an RNA sequencing method, for determining the primary RNA sequence and the presence/identification of RNA modifications, is provided comprising the steps of: (i) labeling of the 5′ and/or 3′ end of the RNA; (ii) random degradation of the RNA; (iii) optionally, physical separation of resultant RNA fragments based on 5′ and 3′ end labeling; (iv) separation and detection of the resultant RNA fragment properties; and (v) data analysis resulting in sequence/modification identification.

In an embodiment, an RNA sequencing method, for determining the primary RNA sequence and the presence/identification of RNA modifications, is provided comprising the steps of: (i) treatment of RNA to be sequenced with N-cyclohexyl-N′-(2-morpholinoethyl)-carbodiimide metho-p-toluenesulfonate (CMC); (ii) affinity labeling of the 5′ and/or 3′ end of the RNA; (iii) random degradation of the RNA into mass ladders; (iv) optionally, physical separation of resultant RNA fragments based on an affinity interaction; (v) measurement of resultant RNA fragments using reverse-phase high performance liquid chromatography (HPLC) or capillary electrophoresis (CE) or other separation methods coupled with mass spectrometry; and (v) MS data analysis resulting in sequence/modification identification.

In specific aspects, the 5′ and 3′ end of the RNA are labeled with affinity-based moieties and/or size shifting moieties. In another aspect, the fragment properties are detected through the use of one or more separation methods including, for example, high performance liquid chromatography, capillary electrophoresis coupled with mass spectrometry.

A hydrophobic end-labelling strategy was used via introducing 2-D mass-retention time (RT) shifts for ladder identification. Specifically, mass-RT labels were added to the 5′ and/or 3′ end of the RNA to be sequenced, and at least one of these moieties results in a retention time shift to longer times, causing all of the 5′ and/or 3′ ladder fragments to have a markedly delayed RT, which clearly distinguished the 5′ ladder from the 3′ ladder. The hydrophobic label tags not only result in mass-RT shifts of labelled ladders, making it much easier to identify each of the 2-D mass ladders needed for LC-MS sequencing of RNA and thus simplifying base-calling procedures, but labelled tags also inherently increase the masses of the RNA ladder fragments so that the terminal bases can even be identified, thus allowing the complete reading of a sequence from one single ladder, rather than requiring paired-end reads.

In certain aspects of the invention, the RNA sequencing method is based on the formation and sequential physical separation of two ladder pools of degraded RNA fragments, referred to herein as 5′ and 3′ ladder pools, which are then subjected to LC/MS for HPLC and MS determination of the RNA sequence as well as the presence of RNA modifications. The physical separation of the 5′ and 3′ ladder pools can be accomplished through the use of a variety of different molecular affinity interactions, such as for example, the affinity of biotin for streptavidin.

In one aspect, the RNA sequencing method disclosed herein comprises the steps of: (i) affinity labeling of the 5′ and/or 3′ end of the RNA molecules; (ii) random degradation of the labeled RNA; (iii) 5′ and/or 3′ end labeled fragment separation based on the affinity labeling; and (iv) sequential performance of liquid chromatography HPLC with high-resolution mass spectrometer (MS) for sequence/modification identification.

In a specific aspect, the method consists of (i) chemical labeling of 5′ and/or 3′ RNA ends for physical separation of ladder fragments based on a biotin/streptavidin affinity (ii) formic acid-mediated RNA degradation, (iii) physical separation of 5′ and/or 3′ labeled RNA (iv) high-performance liquid chromatography (HPLC)-mediated separation of fragments, (v) sequential ESI-Quadrupole-Time-of-Flight (Q-TOF)-MS-based mass detection, and (iv) data analysis based on a simple computational algorithm that extracts, aligns and processes relevant mass peaks from the mass spectrum.

In another specific example, the method consists of (i) 5′ end chemical labeling of RNA with a bulky hydrophobic tag, like Cy3, which is designed to increase the size of the RNA fragment to increase retention time, and 3′ end labeling with an affinity tag like biotin, or vice versa, thus permitting sequence identification without the need for physical separation (ii) formic acid-mediated RNA degradation, (iii) high-performance liquid chromatography (HPLC)-mediated separation of fragments, and sequential ESI-Quadrupole-Time-of-Flight (Q-TOF)-MS-based mass detection, and (iv) data analysis based on a simple computational algorithm that extracts, aligns and processes relevant mass peaks from the mass spectrum.

Further details and aspects of exemplary embodiments of the disclosure are described in more detail below with reference to the appended figures. Any of the above aspects and embodiments of the disclosure may be combined without departing from the scope of the disclosure.

### BRIEF DESCRIPTION OF THE DRAWINGS

Various embodiments of the present methods for RNA sequencing and modification identification are described herein with reference to the drawings wherein:

FIG. 1 shows workflow for introducing a biotin label to the 3′ end and 5′ end of RNA, respectively, followed by acid degradation and biotin/streptavidin capture release to generate mass ladders for direct sequencing by LC-MS;

FIG. 2 shows secondary cloverleaf structure of tRNA^{Phe} from yeast, T₁ ribonuclease only cut single stranded RNA G position;

FIG. 3 shows partial T₁ ribonuclease digestion of tRNA to generate three overlapping fragments;

FIG. 4 demonstrates 3′ tRNA portion labeling using T₄ ligase with 5′-adenylated biotin-methyl-ddC as substrate and subsequent 3′ ladder formation after streptavidin fishing, acid degradation, and LC/MS;

FIG. 5 shows middle portion of tRNA labeling using T4 polynucleotide kinase (PNK) followed by thio transfer with Biotin (long arm) Maleimide and subsequent 5′ ladder formation after streptavidin fishing, acid degradation, and LC/MS;

FIG. 6 demonstrates 5′ tRNA portion labeling using 5′ phosphatase to remove 5′ phosphate group and replace with 5′-OH group, with ladder generation following previous 5′ procedure;

FIG. 7 shows LC/MS sequence determination of a bead separated 5′ labeled RNA;

FIG. 8 demonstrates direct LC-MS sequencing of 5′-biotin labeled 21-nt RNA before isolation using the computational algorithm defined by their mass, chromatographic RT and abundance; the degradation time is 15 min;

FIG. 9 shows MALDI-TOF mass spectra of 3′-end biotin labeling reaction products with the starting molecule 21-nt RNA producing m/z 6784 and the 3′-end biotin labeled 21-nt RNA producing m/z 7541, respectively;

FIG. 10 shows MALDI-TOF mass spectra of 5′-end biotin labeling reaction products with the starting molecule 21-nt RNA producing m/z 6784 and the 3′-end biotin labeled 21-nt RNA producing m/z 7353, respectively;

FIG. 11 shows direct LC-MS sequencing of 5′-biotin labeled 21-nt RNA using the computational algorithm defined by their mass, chromatographic RT and abundance, without bead separation; the degradation time is 5 min;

FIG. 12. Shows workflow without bead-aided physical separation by introducing a biotin label to the 3′ end and a hydrophobic Cy3 tag to the 5′ end of RNA, respectively, followed by acid degradation to generate mass ladders for direct sequencing by LC-MS;

FIG. 13. Depicts known masses of modified ribonucleosides;

FIG. 14A. HPLC profile showing the high yield of labeling of a 21 nt RNA with 5′-sulfo-Cy3. FIG. 14B. the structure of A(5′)pp(5′)Cp-TEG-biotin-3′ which is synthesized to afford higher 3′-labeling efficiency;

FIG. 15A. Simultaneous sequencing of 5 RNAs after biotin labeling at the 3′ end and sulfo-Cy3 labeling at the 5′ end. FIG. 15B Simultaneous sequencing of 12 RNAs after biotin labeling at the 3′ end and sulfo-Cy3 labeling at the 5′ end. *Retention time was adjusted by adding 2 min for each ladder for better visualization of the different sequence readouts;

FIG. 16A. Method for introducing a biotin label to the 3′ end of RNA. FIG. 16B. Separation of the 3′ladder from the 5′ ladder and other undesired fragments on a mass-retention time (RT)-plot based on systematic changes in RT of 3′-biotin-labeled mass-RT ladders of RNA #1. The sequences were de novo generated automatically by an algorithm described in the SI; FIG. 16C. Simultaneous sequencing of two RNAs of different lengths (RNA #1 and RNA #2) after 5′biotin labeling. The sequences presented were manually acquired based on the mass-RT ladders identified from the automatically-generated filtered and processed data;

FIG. 17A. General strategy to differentiate two series of ladder fragments (5′ vs. 3′) from each other by introducing a hydrophobic cyanine 3 (Cy3) to the 5′ end and biotin to the 3′ end, respectively, of any RNA. FIG. 17B. Mass-RT plot of a sample containing all the ladder fragments needed for sequencing from 5′-Cy3-labeled and 3′-biotin-labeled RNA #1; Differentiation of the ladders can occur due to significant changes in the RTs afforded by the two tags. The sequence was manually read from both mass-RT ladders identified from the filtered and processed data from the automatically-generated mass-RT plot;

FIG. 18A. HPLC profile for the high yield of labeling of RNA #11 with sulfo-Cy3 at the 5′end. FIG. 18B. HPLC profile for the high yield of labeling of RNA #11 with biotin at the 3′ end using A(5′)pp(5′)Cp-TEG-biotin-3′. FIG. 18C. Structure of sulfo-Cy3 maleimide and A(5′)pp(5′)Cp-TEG-biotin-3′, applied to achieve a higher labeling efficiency at the 5′ and 3′ ends, respectively;

FIG. 19A. chemical conversion of pseudouridine (ψ) by reaction with N-cyclohexyl-N′-(2-morpholinoethyl)-carbodiimide metho-p-toluenesulfonate (CMC) to form CMC-ψ, shifting CMC-ψ-containing mass-RT ladders in both mass and RT compared to mass-RT ladders containing unconverted ψ. FIG. 19B. sequencing of RNA #12, which contains 1 ψ. The CMC-converted ψ(depicted as ψ*) results in a shift in both RT and mass, allowing facile identification and location of ψ at this position due to a single drastic jump in the mass-RT ladder. FIG. 19C. sequencing of RNA #13, which contains 2 ψ. Each of the CMC-converted ψ (depicted as ψ*) results in a drastic jump in the mass-RT ladder, corresponding to the locations of the ψ in the RNA sequence. For ease of visualization, only the sequences of 5′ mass-RT ladders are presented;

FIG. 20. Simultaneous sequencing of a mixed sample containing 12 RNAs with either a single FIG. 20A. biotin label at the 3′ end or a FIG. 20B. sulfo-Cy3 labeling at the 5′ end of each RNA (RNA #12 was only in the 3′-biotin-labeled sample mixture, and thus FIG. 20A contains one additional sequence compared to FIG. 20B. RT was normalized for ease of visualization (Methods);

FIG. 21A-B. LC/MS sequencing and quantification. FIG. 21A. Sequencing of a mixture containing 20% m⁵C modified RNA (RNA #14) and 80% of non-modified RNA (RNA #3). Both curves share the identical sequence until the first C is reached; the RT of the m⁵C-terminated ladder fragment was shifted up (due to the hydrophobicity increase from the methyl group) and the mass slightly increased (due to the 14 Da mass increase from the additional methyl group) compared to its non-modified counterpart. Both sequences were read manually from mass-RT ladders identified from the algorithm-processed data. FIG. 21B. Quantifying the stoichiometry/percentage of RNA with modifications vs. its canonical counterpart RNA. The relative percentages are quantified by integrating the extracted ion current (EIC) of different labeled product species, and they match well with ratios of the absolute amounts initially used for labeling these RNA samples, i.e., percentages of m⁵C modified RNA in the mixed samples were 10%, 20%, 30%, 40%, 50% and 100%, respectively, which was calculated from their mole ratios initially used for labeling;

FIG. 22A. Unlabeled 3′ and 5′mass ladders of a synthetic, unmodified A10 (10-mer of polyadenine) sequence generated in silico. FIG. 22B. 5′ and 3′mass ladders of a synthetic, 5′-Cy3-labeled A10 (10-mer of polyadenine) sequence generated in silico;

FIG. 23. Mass-RT plot of a sample containing complete sets of ladder fragments from 5′-sulfo-Cy3-labeled RNA #1 and its 3′-unlabeled ladder fragments containing manually-read sequence data from automatically-generated mass-RT plots containing mass-RT ladders identified from filtered and processed data;

FIG. 24. HPLC profile of the crude products after conversion of pseudouridine (w) to its N-cyclohexyl-N′-(2-morpholinoethyl)-carbodiimide metho-p-toluenesulfonate (CMC) adduct in FIG. 24A a 20 nt RNA (RNA #12) containing 1 ψ base and FIG. 24B a 20 nt RNA (RNA #13) containing 2 ψ bases; and

FIG. 25. Utilization of internal fragments without either original 5′ or 3′end to fill gaps in the 5′ladders ladder before reporting the final sequence of a 20 nt RNA, thus increasing the method's accuracy by combining three pieces of information including FIG. 25A the 5′ladder, FIG. 25B the 3′ladder, and FIG. 25C internal fragments whose observed masses match with a list of theoretical masses from the proposed sequence.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The current disclosure is related to a direct, liquid-chromatography-mass spectrometry (herein referred to as LC-MS) based RNA sequencing method which can be used to directly sequence RNA without cDNA synthesis, simultaneously determine the nucleotide sequence of RNA molecules with single nucleotide resolution as well as detection of the presence of target RNA modifications. The disclosed method can be used to determine the type, location and quantity of modifications within the RNA sample. The RNA to be sequenced may be a purified RNA sample of limited diversity, as well as samples of RNA containing complex mixtures of RNA, such as RNA derived from a biological sample. Such techniques can be used to determine the nucleotide sequence of an RNA molecule and to advantageously correlate the biological functions of any given RNA molecule with its associated modifications.

As used herein, ribonucleic acid (RNA) refers to oligoribonucleotides or polyribonucleotides as well as analogs of RNA, for example, made from nucleotide analogs. The RNA will typically have a base moiety of adenine (A), guanine (G), cytosine (C) and uracil (U), a sugar moiety of a ribose and a phosphate moiety of phosphate bonds. RNA molecules include both natural RNA and artificial RNA analogs. The RNA can be synthetic or can be isolated from a particular biological sample using any number of procedures which are well known in the art, wherein the particular chosen procedure is appropriate for the particular biological sample. RNA samples include for example, mRNA, tRNA, antisense-RNA, and siRNA, to name a few. No limitations are imposed on the base length of RNA. The LC-MS-based sequencing methods disclosed herein enable the sequencing of not only purified RNA samples, but also more complicated RNA samples containing mixtures of different RNAs.

In a specific embodiment, the structure of synthetic oligoribonucleotides of therapeutic value can be determined using the sequencing methods disclosed herein. Such methods will be of special valuable to those engaged in research, manufacture, and quality control of RNA-based therapeutics, as well as the regulatory entities. Incorporation of structural modifications into synthetic oligoribonucleotides has been a proven strategy for improving the polymer's physical properties and pharmacokinetic parameters. However, the characterization and the structure elucidation of synthetic and highly-modified oligonucleotides remains a significant hurdle.

In addition to sequencing of RNA, the methods disclosed herein may be used to determine the sequence of DNA. As used herein, deoxynucleic acid (DNA) refers to oligonucleotides or polynucleotides as well as analogs of DNA, for example, made from nucleotide analogs. The DNA will typically have a base moiety of adenine (A), guanine (G), cytosine (C) and thymine (T), a sugar moiety of a deoxyribose and a phosphate moiety of phosphate bonds. DNA molecules include both natural DNA and artificial DNA analogs. The DNA can be synthetic or can be isolated from a particular biological sample using any number of procedures which are well known in the art, wherein the particular chosen procedure is appropriate for the particular biological sample. DNA samples include for example, genomic DNA and mitochondrial DNA, to name a few. No limitations are imposed on the base length of DNA. With proper enzymatic and/or chemical degradation, the LC-MS-based sequencing methods disclosed herein enable the sequencing of not only purified DNA samples, but also more complicated DNA samples containing mixtures of different DNAs. In non-limiting embodiments of the invention, enzymatic degradation of the DNA can be achieved using DNA restriction endonucleases.

In one aspect, the sequencing method of the invention comprises the steps of: (i) affinity labeling of the 5′ and 3′ end of the RNA sample to facilitate subsequent separation of the 5′ and 3′ end labeled RNA pools; (ii) random non-specific cleavage of the RNA; (iii) physical separation of resultant target RNA fragments using affinity based interactions; (iv) LC/MS measurement of resultant mass ladders with liquid chromatography (LC) and high resolution mass spectrometry (MS); and (iv) sequence generation and modification analysis.

In an embodiment, an RNA sequencing method, for determining the primary RNA sequence and the presence/identification of RNA modifications, is provided comprising the steps of: (i) labeling of the 5′ and/or 3′ end of the RNA; (ii) random degradation of the RNA; (iii) optionally, physical separation of resultant RNA fragments based on 5′ and 3′ end labeling; (iv) separation and detection of the resultant RNA fragment properties; and (v) data analysis resulting in sequence/modification identification.

In an embodiment, an RNA sequencing method, for determining the primary RNA sequence and the presence/identification of RNA modifications, is provided comprising the steps of: (i) treatment of RNA to be sequenced with N-cyclohexyl-N′-(2-morpholinoethyl)-carbodiimide metho-p-toluenesulfonate (CMC); (ii) affinity labeling of the 5′ and 3′ end of the RNA; (iii) random degradation of the RNA; (iv) optionally, physical separation of resultant RNA fragments based on an affinity interaction; (v) measurement of resultant RNA fragments using reverse-phase high performance liquid chromatography (HPLC) or capillary electrophoresis (CE) or other separation methods coupled with mass spectrometry; and (v) MS data analysis resulting in sequence/modification identification.

In a specific aspect, the method consists of (i) chemical labeling of 5′ and 3′ RNA ends for physical separation of ladder fragments based on a biotin/streptavidin affinity (ii) formic acid-mediated RNA degradation, (iii) physical separation of 5′ and 3′ labeled RNA (iv) high-performance liquid chromatography (HPLC)-mediated separation of fragments, (v) sequential ESI-Quadrupole-Time-of-Flight (Q-TOF)-MS-based mass detection, and (iv) data analysis based on a simple computational algorithm that extracts, aligns and processes relevant mass peaks from the mass spectrum.

In another specific example, the method consists of (i) 5′ end chemical labeling of RNA with a bulky hydrophobic tag, like Cy3, which is designed to increase the size of the RNA fragment to increase retention time, and 3′ end labeling with an affinity tag like biotin, or vice versa, thus permitting sequence identification without the need for physical separation (ii) formic acid-mediated RNA degradation, (iii) high-performance liquid chromatography (HPLC)-mediated separation of fragments, and sequential ESI-Quadrupole-Time-of-Flight (Q-TOF)-MS-based mass detection, and (iv) data analysis based on a simple computational algorithm that extracts, aligns and processes relevant mass peaks from the mass spectrum.

Such, non-limiting computational algorithms that may be used in the practice of the invention include, for example, those disclosed in PCT/US19/33895 filed May 24, 2019 which is incorporated herein by reference in its entirety.

Although, the sequencing method disclosed herein is generally based on the formation and sequential physical separation of the two 5′ and 3′ ladder pools of degraded target RNA fragments for MS analysis, the physical separation of ladder pools is not a required step as the labeled RNA degraded fragments will have a retention time shift as compared to unlabeled RNA degraded fragments which can be differentiated in 2-dimensional mass-retention time plot after the LC/MS step.

As one step in the sequence method disclosed herein, the RNA to be sequenced is subjected to random controlled degradation. As used herein, the terms degradation and cleavage may be used interchangeably. It is understood that the degradation, or cleavage, of RNA refers to breaks in the RNA strand resulting in fragmentation of the RNA into two or more fragments. In general, such fragmentation for purposes of the present disclosure are random. However, site specific fragmentation may also be employed. RNA's natural tendency to be degraded can be advantageously used to generate a sequence ladder, i.e., a mass latter, for subsequent sequence determination via liquid chromatography-mass spectrometry (LC-MS). By controlling the timing of exposure to a degradation reagent, single but randomized cleavage along the target RNA molecule backbone may be achieved, thus simplifying downstream MS data analysis.

In one aspect, the target RNA molecule is exposed to random chemical cleavage to form ladder pools of degraded target RNA fragments. In a preferred embodiment chemical cleavage is accomplished through use of formic acid. Formic acid degradation is preferred because its boiling point is approximately 100° C. like water and the formic acid can be easily remove it e.g., by lyophilizer or speedvac. Such cleavage is designed to cleave the RNA molecule at its 5′-ribose positions throughout the molecule. In addition to formic acid degradation, alkaline degradation may also be used. For example, the following alkaline buffers may be used to degrade the RNA sample: 1× Alkaline Hydrolysis Buffer (e.g., 50 mM Sodium Carbonate [NaHCO₃/Na₂CO₃] pH 9.2, 1 mM EDTA; or the Alkaline Hydrolysis Buffer supplied with Ambion's RNA Grade Ribonucleases). In addition to chemical cleavage, RNAs may be subjected to enzymatic degradation. Enzymes that may be used to degrade the RNA include for example, Crotalus phosphodiesterase I, bovine spleen phosphodiesterse II and XRN-1 exoribonucease. Such RNA degradation treatment is carried out under conditions where a desired single cleavage event occurs on the RNA molecule resulting in a pool of differently sized RNA fragments resulting in a complete ladder.

As a further step in the sequencing method disclosed herein, the ends of the RNA fragments are labeling to provide affinity interactions that can be utilized to provide a means for separation of the fragmented 5′ or 3′ labeled fragment pools within the cleavage mixture. Such affinity interactions are well known to those skilled in the art and included, for example, those interactions based on affinities such as those between antigen and antibody, enzyme and substrate, receptor and ligand, or protein and nucleic acid, to name a few. Labeling of the 5′ and 3′ ends of the fragmented RNA for use in affinity separation may be achieved using a variety of different methods well known to those skilled in the art. Such labeling is designed to achieve separation of fragmented RNA for subsequent MS analysis. RNA end-labeling may be performed before or after the chemical cleavage of the RNA.

In a preferred embodiment, the biotin/streptavidin interaction may be utilized to enrich for the ladder RNA fragments. In yet another preferred embodiment, the poly (A) oligonucleotide/dT interaction may be used to separate fragmented RNA. In instances where the end of the RNA is labeled with a biotin moiety, streptavidin beads may be used to purify the desired RNA ladder fragments. Alternatively, where the RNA has been labeled with a poly (A) DNA oligonucleotide, oligopoly (dT) immobilized beads such as (dT) 25-cellulose beads (New England Biolabs) may be used to enrich for the RNA fragments. The choice of chromatography material will be dependent on the 5′ and 3′ RNA labeling used and selection of such chromatography/separation material is well known to those skilled in the art.

As one example, the 3′ and 5′ RNA ends may be labeled with biotin for subsequent separation of RNA fragments based on the biotin/streptavidin interaction through use of streptavidin beads. In yet another aspect, short DNA adapters may be ligated to each end of the RNA sample. The 3′ end of the RNA may be ligated to a 5′ phosphate-terminated, pentamer-capped photocleavable poly(A) DNA oligonucleotide with T4 RNA ligase to form a phosphodiester-linked RNA-DNA hybrid. The 5′ end of the RNA-DNA hybrid may then be ligated to 5′ biotinylated DNA after phosphorylation via T4 polynucleotide kinase using T4 RNA ligase.

In a specific embodiment, two short DNA adapters are ligated to each end of the RNA sample, to physically select the desired fragment into either the 5′ or 3′ ladder pool from the undesired fragments with more than one phosphodiester bond cleavage in the crude degraded product mixture, followed by a lengthened formic acid degradation time resulting in most of the RNA sample being degraded, most of which turn into the desired fragments needed to obtain a complete sequence ladder. The 3′ end of the RNA sample is ligated to a 5′-phosphate-terminated, pentamer-capped photocleavable poly (A) DNA oligonucleotide with T4 RNA ligase 1 (New England Biolabs) to form a phosphodiester-linked RNA-DNA hybrid. Likewise, the 5′ end of the RNA-DNA hybrid is ligated to 5′-biotinylated DNA after phosphorylation via T4 polynucleotide kinase with the same ligase. The resulting 5′ DNA-RNA-DNA-3′ hybrid is treated with formic acid for approximately 5-15 min. Following formic acid treatment, streptavidin-coupled beads (ThermoFisher Scientific) can be used to isolate the 5′ ladder fragment pool followed by oligomer-release for subsequent LC/MS analysis. Similarly, oligopoly (dT) immobilized beads such as (dT) 25-Cellulose beads (New England Biolabs) can be used to enrich the 5′ ladder, which can then be eluted for LC/MS analysis after photocleavage by UV light (300-350 nm). Only the RNA section of the hybrid will be hydrolyzed, while the DNA section will remain intact as DNA lacks the 2′-OH group. In a specific embodiment, a biotin tag is added via a two-step reaction, at each end of the RNA sample. As a first step, a thiol-containing phosphate is introduced at the 5′-end by reacting T4 polynucleotide kinase with adenosine 5′[γ-thio]triphosphate (ATP-γ-S) to add a thiophosphate to the 5′ hydroxyl group of the to-be-sequenced RNA and then a conjugation addition is made between the resultant thiolphosphorylated RNA and the biotin (Long Arm) Maleimide (Vector Laboratories, USA), which is designed for biotinylating proteins, nucleic acids, or other molecules containing one or more thiol groups. The resulting 5′-biotinylated-RNA is then treated with formic acid, similar to the previous procedure (13). After acid degradation, streptavidin-coupled beads (Thermo Fisher Scientific, USA) are used to single out the 5′ ladder pool, which will be released for subsequent LC/MS analysis after breaking the biotin-streptavidin interaction. Although, the sequencing methods disclosed herein are generally based on the formation and sequential physical separation of 5′ and 3′ ladder pools of degraded target RNA fragments for MS analysis, the physical separation of ladder pools is not a required step. The labeled RNA degraded fragments will have a retention time shift as compared to unlabeled RNA degraded fragments which can be differentiated via the LC/MS step. In a specific embodiment, to increase the retention time shift, the RNA may be labeled with bulky moieties such as, for example, a hydrophobic Cy3 or Cy5 tag or other fluorescent tag. Such a tag is added via a two-step reaction, at the 5′-end of the RNA sample. As a first step, a thiol-containing phosphate is introduced at the 5′-end by reacting T4 polynucleotide kinase with adenosine 5′[γ-thio]triphosphate (ATP-γ-S) to add a thiophosphate to the 5′ hydroxyl group of the to-be-sequenced RNA and then a conjugation addition is made between the resultant thiolphosphorylated RNA and the Cy3 or Cy5 Maleimide (Tenova Pharmaceuticals, USA), which is designed for biotinylating proteins, nucleic acids, or other molecules containing one or more thiol groups. After 3′ end biotin labeling and acid degradation, the resultant two-end-labeled RNA is directly subjected for LC/MS without any affinity-based physical separation.

For 3′ end labeling, after isolating the 5′ ladder pool (which will be analyzed by LC/MS) in case affinity tags were used, the remaining residue, which contains the 3′ ladder pool with all of the original 3′-hydroxyl groups, will be subjected to 3′ end labeling. For this purpose, biotinylated cytidine bisphosphate (pCp-biotin) is activated by adenylation using ATP and Mth RNA ligase to produce AppCp-biotin. Then the members of the 3′ ladder pool with a free 3′ terminal hydroxyl are then ligated to the activated 5′-biotinylated AppCp via T4 RNA ligase, thus resulting in the 3′ end of each sequence in the 3′ ladder pool becoming biotin-labeled. Similarly, streptavidin-coupled beads are used to isolate the 3′ ladder pool, which will be released for subsequent LC/MS analysis (separate from the 5′ ladder pool) after breaking the biotin-streptavidin interaction.

Once separation of RNA fragment pools is performed, the RNA fragments can be analyzed by any of a variety of means including liquid chromatography coupled with mass spectrometry, or capillary electrophoresis coupled with mass spectrometry or other methods known in the art. Preferred mass spectrometer formats include continuous or pulsed electrospray (ESI) and related methods or other mass spectrometer that can detect RNA fragments like MALDI-MS. HPLC-MS measurements can be performed using high resolution time-of-flight or Orbitrap mass spectrometers that have a mass accuracy of less than 5 ppm. The use of such mass spectrometers facilitates accurate discernment between cytosine and uridine bases in the RNA sequence. In one aspect of the invention, the mass spectrometer is an Agilent 6550 and 1200 series HPLC with a Waters XBridge C18 column (3.5 μm, 1×100 mm). Mobile phase A may be aqueous 200 mM HFIP (1,1,1,3,3,3-Hexafluoro-2-propanol) and 1-3 mM TEA (Triethylamine) at pH 7.0 and mobile phase B methanol. In a specific non-limiting embodiment, the HPLC method for a 20 μL of a 10 μM sample solution was a linear increase of 2%-5% to 20%-40% B over 20-40 min at 0.1 mL/min, with the column heated to 50 or 60° C. Sample elution was monitored by absorbance at 260 nm and the eluate was passed directly to an ESI source with 325° C. drying with nitrogen gas flowing at 8.0 L/min, a nebulizer pressure of 35 psig and a capillary voltage of 3500 V in negative mode.

LC-MS data is converted into RNA sequence information. The unique mass tag of each canonical ribonucleotide and its associated modifications on the RNA molecule, allows one to not only determine the primary nucleotide sequence of the RNA but also to determine the presence, type and location of RNA modifications.

In the event of DNA, LC-MS data is converted into DNA sequence information. The unique mass tag of each canonical deoxynucleotide and its associated modifications on the DNA molecule, allows one to not only determine the primary nucleotide sequence of the DNA but also to determine the presence, type and location of DNA modifications. In a specific embodiment, the raw data derived from LC-MS, which contains the LC/MS data of the desired fragments and/or the undesired fragments is subsequently used for sequence alignment and detection of base modification. In addition to a two-dimensional data analysis which relies on mass and retention times, it is understood that additional types of two- or even three-dimensional data analysis may be performed based on other unique properties of RNA fragments, such as for example, unique electronic or optical signature signals that can be used together with mass for sequence determination.

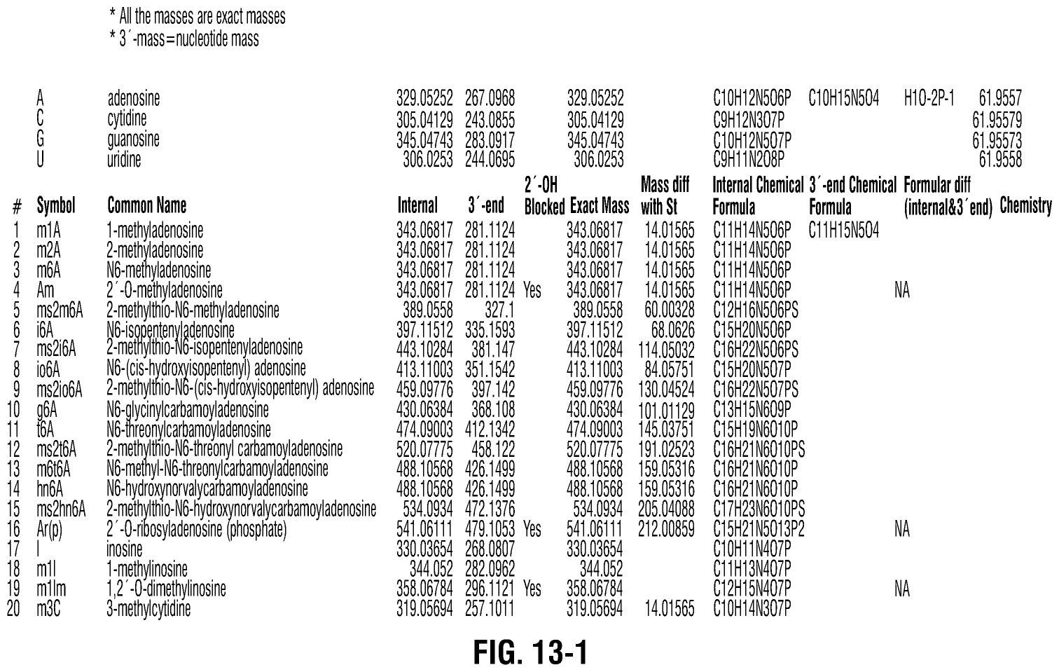

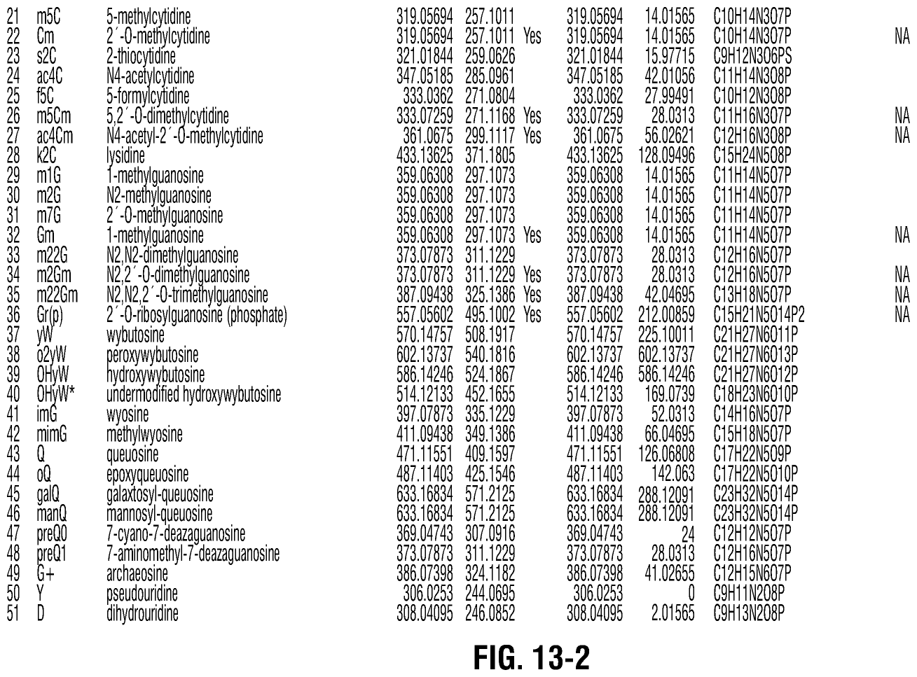

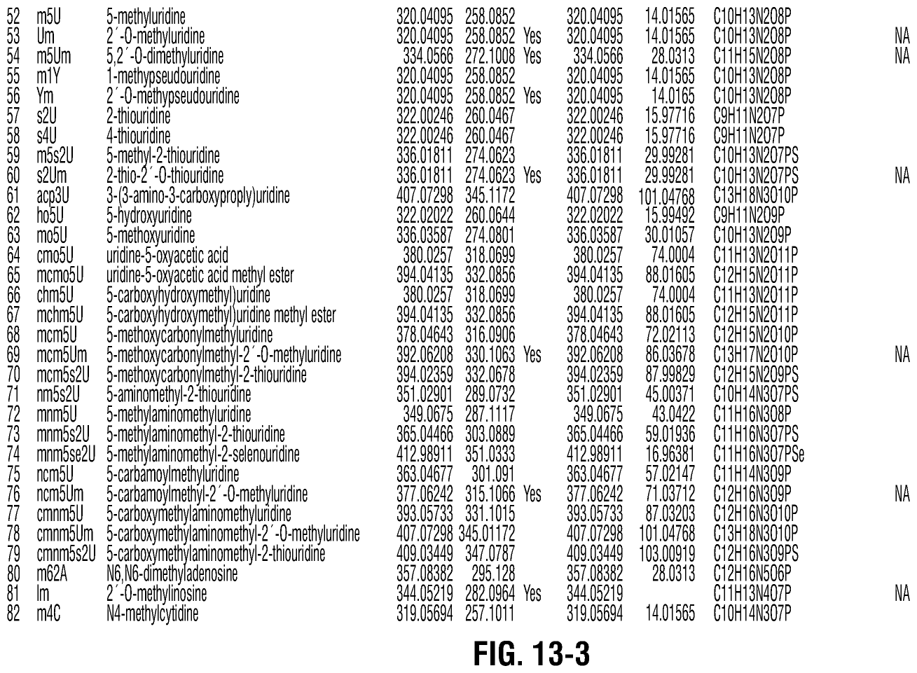

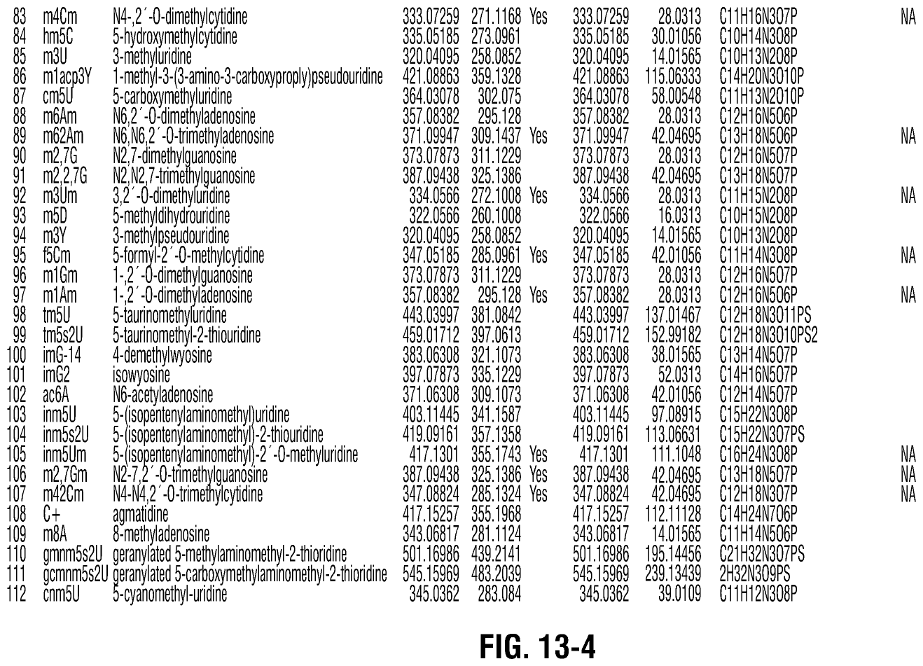

Mass adducts can be removed from the deconvoluted data and the sequences will be predicted/generated using both mass and retention time data. The retention time-coupled mass data for the fragments is analyzed to determine which data points are “valid” and to be used for subsequent sequence determination and which data points are to be filtered out. After data reduction step, the mass difference (m) between two adjacent RNA fragments [m=m (i)−m(i−1), 1<i<n, n=RNA length], where m(i) is the mass of any ladder fragment and m(i−1) is the preceding lower mass ladder fragment, and match such mass differences with the exact masses of known nucleotide fragments to correlate the derived RNA sequencing information based on mass differences to determine the RNA sequence and its modification. As long as the structural modification on an RNA nucleoside is mass-altering, the disclosed sequencing method will permit identification of the RNA sequence and its modification to be identified. The mass of all the known modified ribonucleosides can be conveniently retrieved from known RNA modification databases (12) or through use of the attached FIG. 13.

### 6. Example

It should be understood that the examples and embodiments provided herein are exemplary examples embodiments. Those skilled in the art will envision various modifications of the examples and embodiments that are consistent with the scope of the disclosure herein. Such modifications are intended to be encompassed by the claims. The examples provided herein are included solely for augmenting the disclosure herein and should not be considered to be limiting in any respect.

### Materials and Methods

RNA oligonucleotides listed below were obtained from Integrated DNA Technologies (Coralville, Iowa, USA). RNA strand sequences were as follows:

9-nt RNA: 5′-HO-CGCAU CUGAC UGACC AAAA-OH-3′
20-nt RNA: 5′-HO-AUAGC CCAGU CAGUC UACGC-OH-3′
21-nt RNA: 5′-HO-GCGGA UUUAG CUCAG UUGGG A-OH-3′

Biotinylated cytidine bisphosphate (pCp-biotin), {Phos (H)}C {BioBB}, was obtained from TriLink BioTechnologies (San Diego, Calif., USA). T4 DNA ligase 1, T4 DNA ligase buffer (10×), the adenylation kit including reaction buffer (10×), 1 mM ATP, and Mth RNA ligase were obtained from New England Biolabs (Ipswich, Mass., USA). The 5′ end tag nucleic acid labeling system kit and biotin maleimide were purchased from Vector Laboratories (Burlingame, Calif., USA). The streptavidin magnetic beads were obtained from Thermo Fisher Scientific (Waltham, Mass., USA).

### 3′ End Labeling Method

Adenylation: The following reaction was set up with a total reaction volume of 10 μL in an RNase-free, thin walled 0.5 mL PCR tube: 1× adenylation reaction buffer, 100 μM of ATP, 5.0 μM of Mth RNA ligase, 10.0 μM pCp-biotin, and nuclease-free, deionized water (Thermo Fisher Scientific, USA). The reaction was incubated in a GeneAmp™ PCR System 9700 (Thermo Fisher Scientific, USA) at 65° C. for 1 hour followed by the inactivation of the enzyme Mth RNA ligase at 85° C. for 5 minutes.

Ligation: A 30 μL reaction solution contained 10 μL of reaction solution from the adenylation step, 10× reaction buffer, 5 μM RNA (19-nt, 20-nt or 21-nt, respectively), 10% (v/v) DMSO (anhydrous dimethyl sulfoxide, 99.9%, Sigma-Aldrich, USA), T4 RNA ligase (10 units), and nuclease-free, deionized water. The reaction was incubated for overnight at 16° C. followed by the column purification as follows.

Column Purification: Oligo Clean & Concentrator (Zymo Research, Irvine, Calif., U.S.A.) was used to remove enzymes, free biotin, and short oligos. 100 μL Oligo Binding Buffer was added to a 50 μL sample (20 μL nuclease-free water was added to bring the total sample volume to 50 μL). 400 μL ethanol was added (200 proof, 100%, Decon Labs, USA), mixed the solution briefly by pipetting, and transferred the mixture to a provided column in a collection tube. The sample was then centrifuged at 10,000 rcf for 30 seconds, the flow-through was discarded, and 750 μL DNA Wash Buffer was added to the column. The sample was then centrifuged again at 10,000 rcf for 30 seconds and the flow-through was discarded, followed by centrifugation at maximum speed for 1 minute. The column was transferred to a microcentrifuge tube, and 15 μL nuclease-free water was directly added to the column matrix (with 1 minute of incubation time) and the sample was centrifuged at 10,000 rcf for 30 seconds to elute the oligonucleotide.

The concentration of the purified RNA reported in (ng/4) was measured by a NanoDrop 1000 Spectrophotometer (Thermo Fisher Scientific Waltham, Mass., USA).

The efficiency of biotin labeling to the 3′ or 5′ end of RNA oligo expressed in % was measured by Matrix-assisted laser desorption ionization time-of-flight mass spectrometry (MALDI-TOF MS) by a Voyager-DE Biospectrometry Workstation (Jet Propulsion Laboratory, USA), based on the calculation of peak intensity at mass (m/z) of starting material and mass (m/z) of labeled product.

### 5′ End Labeling Method

Labeling biotin to 5′ end of RNA requires two steps: A thiophosphate is transferred from ATPγS to the 5′ hydroxyl group of the target RNA by T4 polynucleotide kinase (NEB, USA); after addition of biotin maleimide, the thiol-reactive label is chemically coupled to the 5′ end of the target RNA. The experimental protocol is as follows. The following was combined in an RNase-free, thin walled 0.5 mL PCR tube: 10× reaction buffer, 30 μM of RNA (19-nt, 20-nt, or 21-nt, respectively), 0.1 mM of ATPγS, 10 units of T4 polynucleotide kinase, while bring total reaction volume to 10 μL with nuclease-free, deionized water. This sample was mixed and incubated for 30 minutes at 37° C. Then 5 μL of biotin maleimide or Cy3 maleimide (dissolved in 312 μL anhydrous DMF (anhydrous dimethyl sulfoxide, 99.9%, Sigma-Aldrich, USA) was added, mixed, and incubated the sample for 30 minutes at 65° C. Column purification was required as well according to the above-mentioned procedures.

### Acid Hydrolysis Degradation

Direct RNA sequencing relies on generating degradative products, and RNA fragments produced by single scission events can be directly sequenced via observing mass differences between compound masses. Acid hydrolysis can rapidly generate internal fragments by multiple scission events from any starting material, and thus formic acid, especially, is a mild and volatile organic acid used extensively in MS because it has a low boiling point and can therefore be easily removed by lyophilization. RNA samples are biotinylated in one time point or divide each of the RNA sample solution into three smaller equal. Aliquots are degraded by acid degradation using 50% (v/v) formic acid at 40° C. with one for 2 min, one for 5 min, and one for 15 min, and then combine them all together for one LC/MS measurement. The reaction mixture was immediately frozen on dry ice followed by lyophilization to dryness, which was typically completed within 1 h. The dried samples were immediately suspended in 20 μL nuclease-free, deionized water for subsequent biotin/streptavidin capture/release step or stored at −20° C.

### Biotin/Streptavidin Capture/Release Step to Generate LC-MS Sequencing Ladders

Biotin/Streptavidin capture uses streptavidin-coated magnetic beads to bind biotin-labeled RNAs, which are immobilized onto streptavidin coated magnetic beads and drawn to a magnet. Bound RNAs should, therefore, be isolated from non-biotin labeled RNAs and impurities and can be later eluted from the beads for LC-MS sequencing analysis.

200 μL of Dynabeads™ MyOne™ Streptavidin Cl beads (Thermo Fisher Scientific, USA) were prepared by first adding an equal volume of 1× B&W buffer. This solution was vortexed and placed on the magnet for 2 min, followed by discarding of the supernatant. The beads were washed twice with 200 μL of Solution A (DEPC-treated 0.1 M NaOH & DEPC-treated 0.05 M NaCl) and once in Solution B (DEPC-treated 0.1 M NaCl). A final addition of 100 μL of 2× B&W buffer brought the concentration of the beads to 20 mg/mL. An equal volume of biotinylated RNA was added in 1× B&W buffer, incubated the sample for 15 min at room temperature using gentle rotation, placed the tube in a magnet for 2 min, and discarded the supernatant. The coated beads were washed 3 times in 1× B&W buffer and the final concentration of each wash step supernatants was measured by Nanodrop for recovery analysis. For releasing the immobilized biotinylated RNAs, the beads were incubated in 10 mM EDTA (Thermo Fisher Scientific, USA), pH 8.2 with 95% formamide (Thermo Fisher Scientific, USA) at 65° C. for 5 min. Finally, this sample tube was placed in a magnet for 2 min and we collect the supernatant by pipetting.

### LC-MS Analysis

Samples were separated and analyzed on an iFunnel Agilent 6550 Q-TOF coupled to an Agilent 1290 Infinity LC system (Agilent Technologies, Santa Clara, Calif., USA) equipped with a MicroAS autosampler and Surveyor MS Pump Plus HPLC system. All separations were performed using an aqueous mobile phase (A) as 25 mM hexafluoro-2-propanol (HFIP) (Thermo Fisher Scientific, USA) with 10 mM diisopropylamine (DIPA) (Thermo Fisher Scientific, USA) at pH 7.0 and organic mobile phase (B) as methanol across a 50 mm×2.1 mm Xbridge C18 column with a particle size of 1.7 μm (Waters, Milford, Mass., USA). The flow rate was 0.3 mL/min, and all separations were performed with the column temperature maintained at 60° C. Injection volumes were 20 μL, and sample amounts were 15-400 pmol of RNA. Data was recorded in negative polarity. The sample data were acquired using the Agilent Technologies MassHunter LC/MS Acquisition software. To extract relevant spectral and chromatographic information from the LC-MS experiments, Molecular Feature Extraction workflow in MassHunter Qualitative Analysis (Agilent Technologies) was used. This molecular feature extractor algorithm performs untargeted feature finding in the mass and retention time dimensions. In principal any software capable of compound identification could be used. The software settings were varied depending on the amount of RNA used in the experiment. In general, as many identified compounds as possible were included. For samples with low concentrations, profile spectral peaks were filtered using a signal-to-noise ratio (SNR) threshold of 5 and, for more concentrated samples, an SNR threshold of up to 20. The other algorithm settings were as follows: “Small Molecules (chromatographic)” extraction algorithm, charge states from −1 to −15, only loss of hydrogen (—H) ions, “Common Organic Molecules” isotope model, minimum quality score 70 (range 0-100), and minimum ion count 500.

### Results

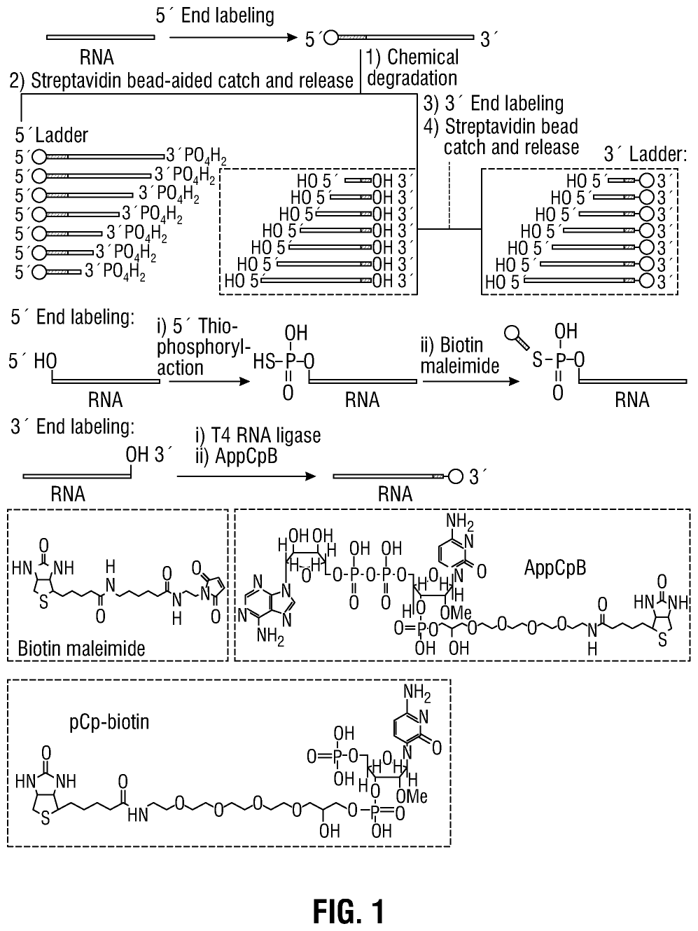

A method is provided for determining the sequence of RNA molecules which is based on the physical separation of two ladders of RNA fragments. The method is designed to prevent any confusion as to which fragment belongs to which ladder by physical separation of two ladders, and the output is expected to contain only one sigmoidal curve rather than two sigmoid curves (which is much more difficult to analyze) in the first-generation method. Another benefit of the sequential separation of two ladders is simplification of the base-calling procedures because after ladder separation, each resultant LC/MS dataset size becomes less than half of the size of the un-separated precursor's dataset. With the help of these two favorable factors, one can sequence more complicated RNA samples with more than one strand while being able to simultaneously analyze their associated modifications. Experiments were designed as shown in FIG. 1 to physically separate the desired fragments into either the 5′ 3′ ladder pool. A biotin tag was added, via a two-step reaction, at each end of the RNA sample through: (i) introduction of a thiol-containing phosphate at the 5′-end by reacting T4 polynucleotide kinase with adenosine 5′-[γ-thio]triphosphate (ATP-γ-S) to add a thiophosphate to the 5′ hydroxyl group of the to-be-sequenced RNA and then (ii) conjugation addition between the resultant thiolphosphorylated RNA and the biotin (Long Arm) Maleimide (Vector Laboratories, USA), which is designed for biotinylating proteins, nucleic acids, or other molecules containing one or more thiol groups. The resulting 5′-biotinylated-RNA is then treated with formic acid, similar to the previous procedure (6). After acid degradation, streptavidin-coupled beads (Thermo Fisher Scientific, USA) are used to single out the 5′ ladder pool, which will be released for subsequent LC/MS analysis after breaking the biotin-streptavidin interaction.

After isolating the 5′ ladder pool (which will be analyzed by LC/MS), the remaining residue, which contains the 3′ ladder pool with all of the original 3′-hydroxyl groups, is subjected to 3′ end labeling. For this purpose, biotinylated cytidine bisphosphate (pCp-biotin) is activated by adenylation using ATP and Mth RNA ligase to produce AppCp-biotin. Then the members of the 3′ ladder pool with a free 3′ terminal hydroxyl are ligated to the activated 5′-biotinylated AppCp via T4 RNA ligase, thus resulting in the 3′ end of each sequence in the 3′ ladder pool becoming biotin-labeled. Similarly, streptavidin-coupled beads can be used to isolate the 3′ ladder pool, which can be released for subsequent LC/MS analysis (separate from the 5′ ladder pool) after breaking the biotin-streptavidin interaction.

A series of synthetic RNA oligos (19-nt, 20-nt, and 21-nt RNA; see Methods for sequences) were designed and synthesized as model RNA oligonucleotides for individual and group test. Biotin-labeled 5′ ends were obtained using the two-step reaction as described above. After acid degradation and bead separation of the 5′ ladder pool for LC/MS analysis, the remaining residue was subjected to 3′-labeling. The members of the 3′ sequence ladder pool were then also biotin end-labeled, streptavidin-captured, and then released for LC/MS analysis as described above.

Experiments were performed focused on tRNA sequencing, as tRNA is very important in protein synthesis and its expression and mutations have major implications in various diseases such as neurological pathologies and cancer development (7-10). However, lack of efficient tRNA sequencing methods has hindered structural and functional studies of tRNA in biological and biochemical processes. tRNA is one class of small cellular RNA for which standard sequencing methods cannot yet be applied efficiently (11); significant obstacles for the sequencing of tRNA include the presence of numerous post-transcriptional modifications and its stable and extensive secondary structure, which can interfere with cDNA synthesis and adaptor ligation. However, as the length of tRNA ranges from 60 to 95 nt, with an average length of 76 nt, it is a very good system to use in the LC/MS-based direct sequencing method disclosed herein.

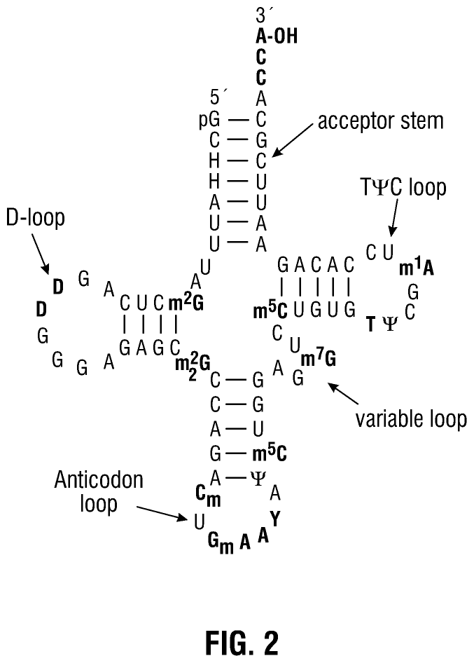

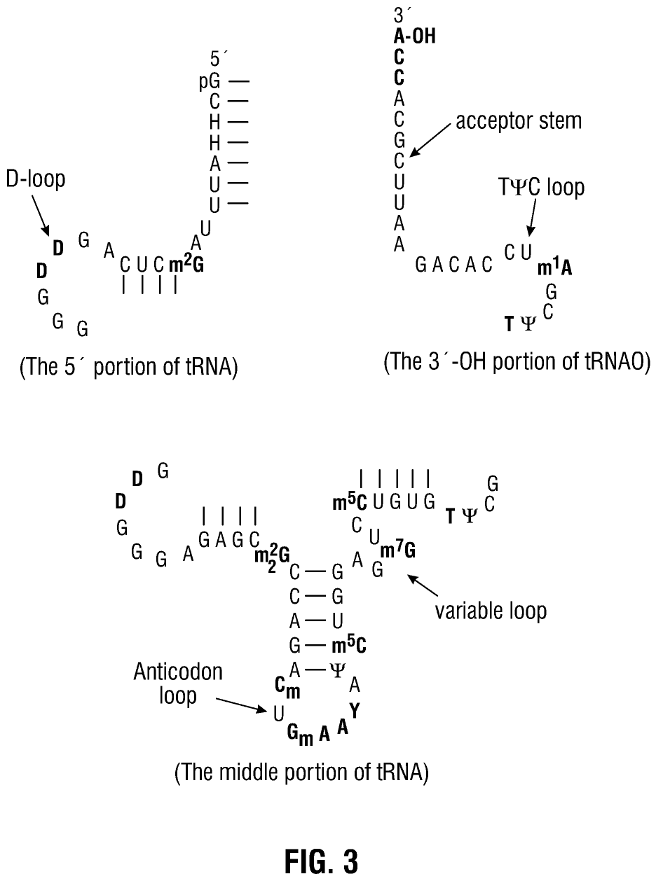

To directly sequence tRNA with the LC/MS-based method, T₁ ribonuclease was used to partially digest the complete tRNA into smaller fragments to allow for successful sequencing. Partial T₁ ribonuclease digestion, which specifically cleaves single-stranded RNA phosphodiester bonds after guanosine residues, producing 3′-phosphorylated ends (FIG. 2), is performed by incubating a phenylalanine specific tRNA at 4-10° C. for 30-60 minutes to obtain three portions of overlapping fragments (FIG. 3): a 5′ portion characterized by sequences containing phosphate groups at both the 5′ and 3′ ends (5′-PO₄_3′ PO₄), an internal portion characterized by sequences containing a hydroxyl group at the 5′ end and a phosphate group at the 3′ end (5′-OH_3′ PO₄), and a 3′ portion characterized by sequences containing hydroxyl groups at both 5′ and 3′ locations (5′-OH_3′ OH). The cloverleaf secondary structure of the tRNA facilitates this digestion step by providing exposed guanosine-residue rich areas for the enzyme to make the cuts.

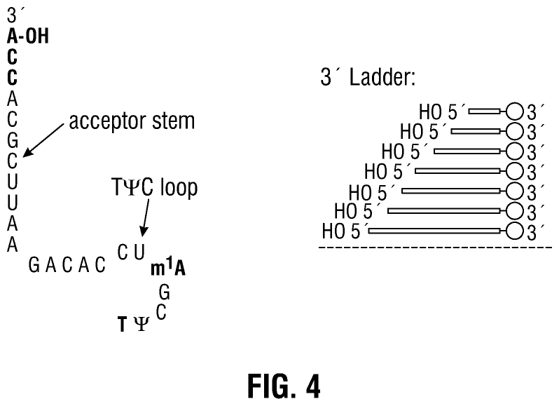

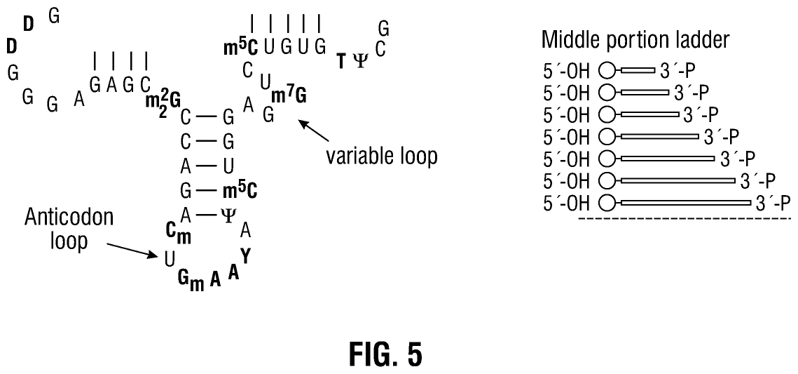

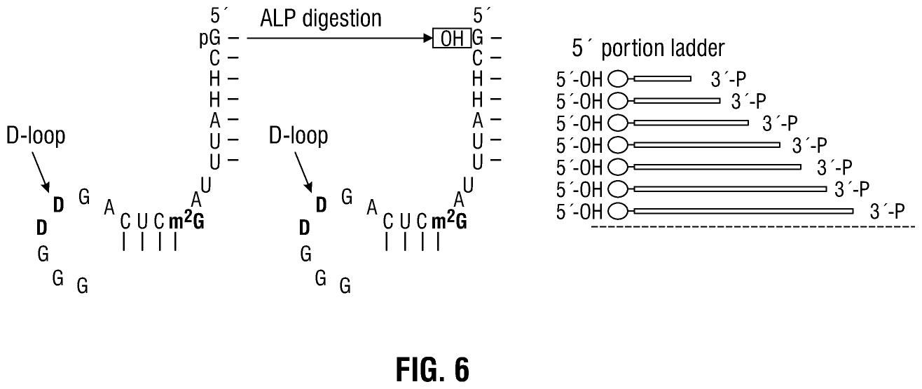

The 3′ tRNA portion, which has an OH group at each of the 3′ and 5′ ends, is labeled using T₄ RNA ligase and 5′-adenylated biotin-methyl-ddC as a substrate. Streptavidin magnetic beads are used to isolate the biotinylated tRNA fragments and acid degradation is performed on the fragments to create the 3′ ladder for sequencing analysis using LC/MS (FIG. 4). For the internal portion of the tRNA (FIG. 5), which are the only sequences that have a 5′-OH after isolation of the above-mentioned 3′-tRNA portion, 5′-labeling is performed by a two-step reaction which was initiated by introducing a thiophosphate to the 5′ hydroxyl group by T4 polynucleotide kinase, followed by a chemical coupling reaction of biotin maleimide to the 5′ end of RNA oligos. The isolation step using streptavidin magnetic beads is again used to single the internal portions out before acid degradation. After acid degradation and LC/MS, the sequences of these internal portion ladder fragments can be obtained by sequence generation and alignment. Next, at the 5′-portion of tRNA fragments (FIG. 6), a 5′ phosphatase removes the 5′ phosphate group and changes it to a hydroxyl group by alkaline phosphatase so that the 5′ end can be labeled using the above-mentioned 5′ end labeling method. Following isolation and acid degradation steps, LC/MS is used to obtain the ladder for the 5′ portion of the tRNA fragment.

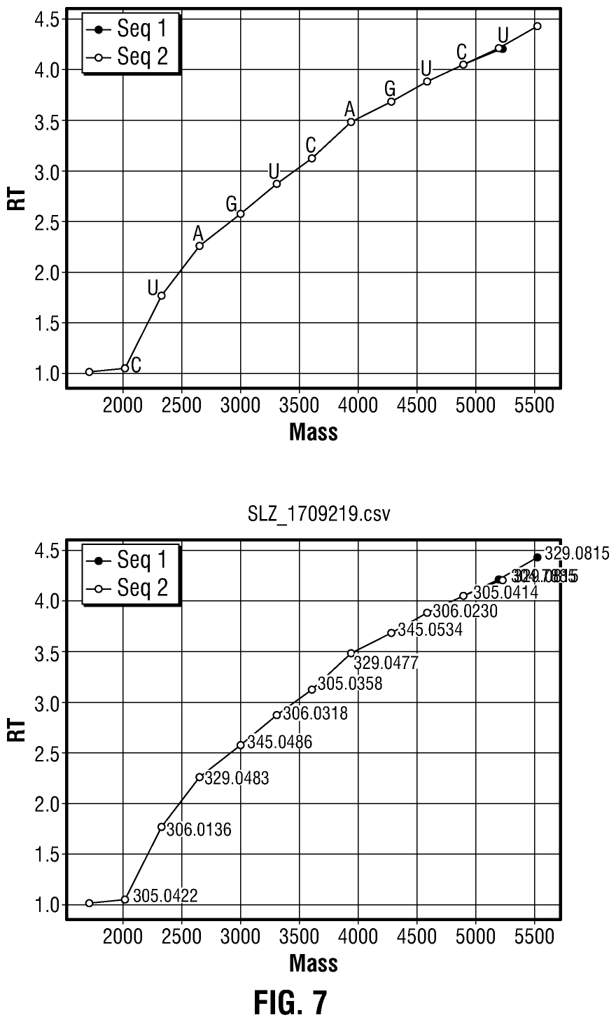

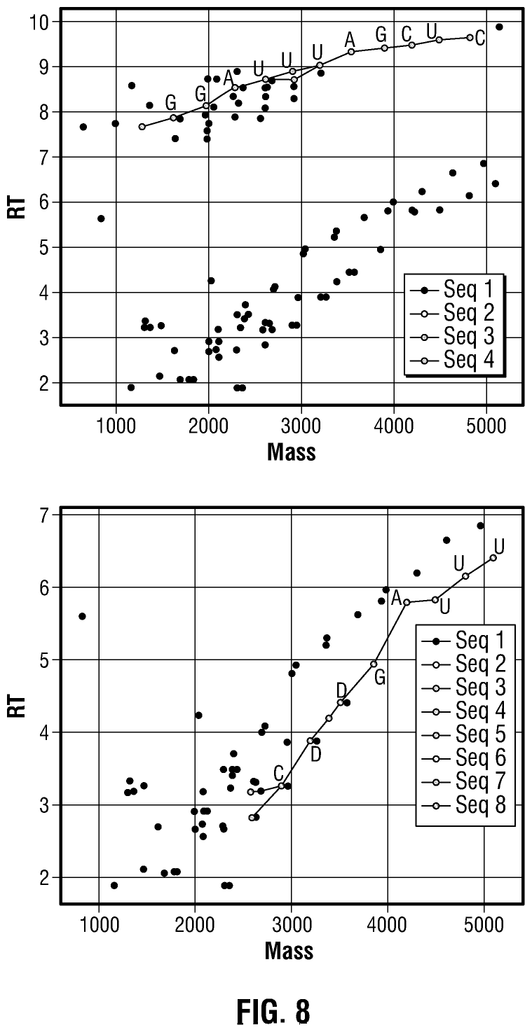

LC/MS data from short oligonucleotides showed that it was possible to observe exactly one sigmoidal curve corresponding to each specific ladder as expected when their masses were plotted against their retention times (t_{R}) (FIG. 7). Even if there are multiple RNA in the mixture consisting of 5′-biotinlyated RNA and non-biotinylated RNA, three different separate sigmoidal curves are observed and their sequences read out readily (FIG. 8).

### Biotin End Labeling Efficiency

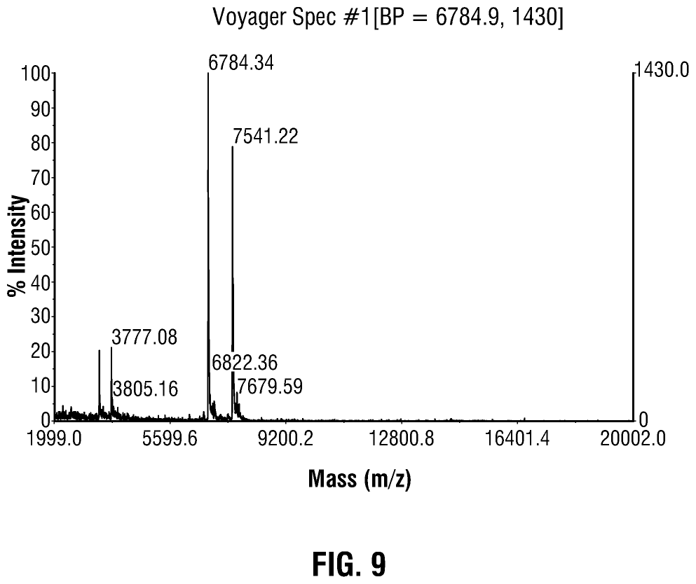

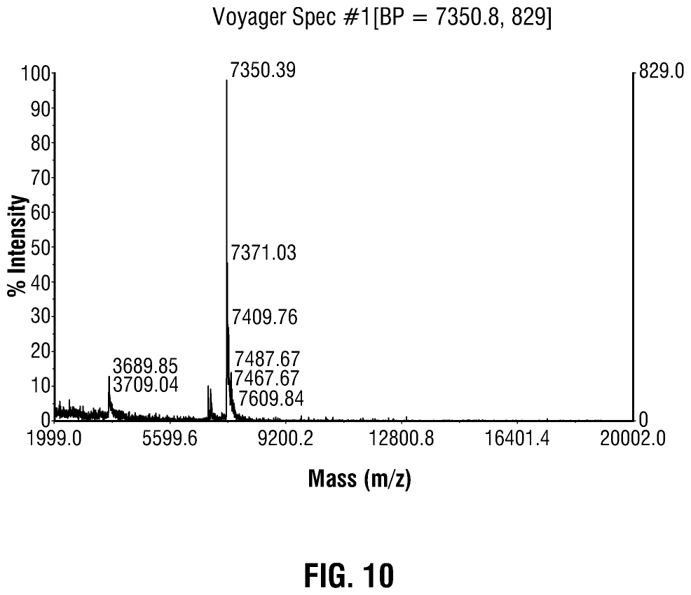

To determine the labeling efficiency, MALDI-TOF MS was applied to estimate the efficiency of biotinylation at 3′ and 5′ end of RNA, respectively (FIG. 9 and FIG. 10), 21-nt RNA as representative data). The efficiency of the labeling reaction was estimated to be 44% and 91% for the 3′ end and 5′ end, respectively, based on the calculation of peak intensity of the mass (m/z) of starting material and the mass (m/z) of labeled product, under the conditions used as descried in the experimental section. The biotin labeled materials are ready to use for acid degradation and biotin/streptavidin capture/release to generate mass ladders for direct sequencing via LC/MS.

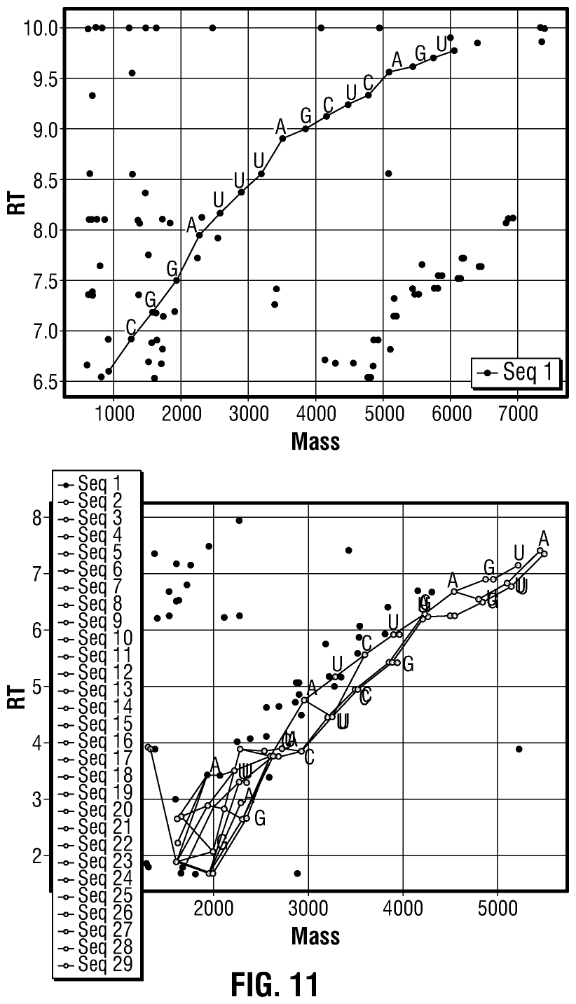

Chromatographic separation of sequence ladders simplified identification of reads in the same orientation. The sequencing reads were defined by their mass, RT, and abundance. The nucleotides (A, G, U, C) were determined by mass differences of two adjacent ladder fragments. Thus, the sequence can be read out very easily. For example, the sequence CGGAUUUAGCUCAGU can be read out automatically from the 5′ to 3′ end for the 5′ end biotin labeled 21-nt RNA (FIG. 11). Together with ladders from the partial unlabeled RNA, the complete sequence of the 21 nucleotides can be read out. Further efforts have been made to read out the complete sequence only for the ladder of labeled RNA, including optimizing experimental conditions such as the biotin/streptavidin capture/release step.

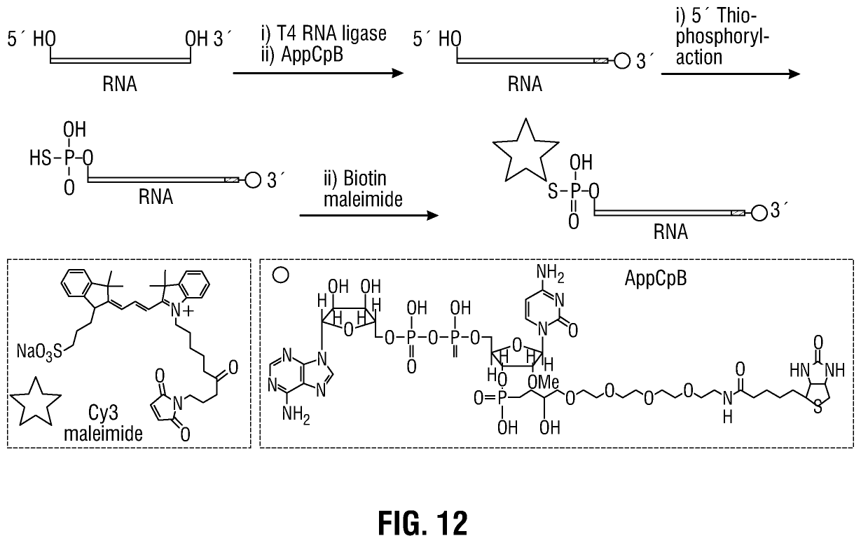

FIG. 12. Demonstrates workflow without bead-aided physical separation by introducing a biotin label to the 3′ end and a hydrophobic Cy3 tag to the 5′ end of RNA, respectively, followed by acid degradation to generate mass ladders for direct sequencing by LC-MS.

The sequencing method described herein provides a tool for RNA sequence analysis through its ability to isolate biotin labeled fragments from two ends, respectively, that can simplify LC/MS data analysis and help read out sequences from each ladder (either 5′ ladder or 3′ ladder) after its physical separation from the other one. This strategy allows one to sequence more complicated RNA samples with more than one RNA strand as well as tRNA, and subsequently analyze their associated modifications simultaneously.

### 7. Example

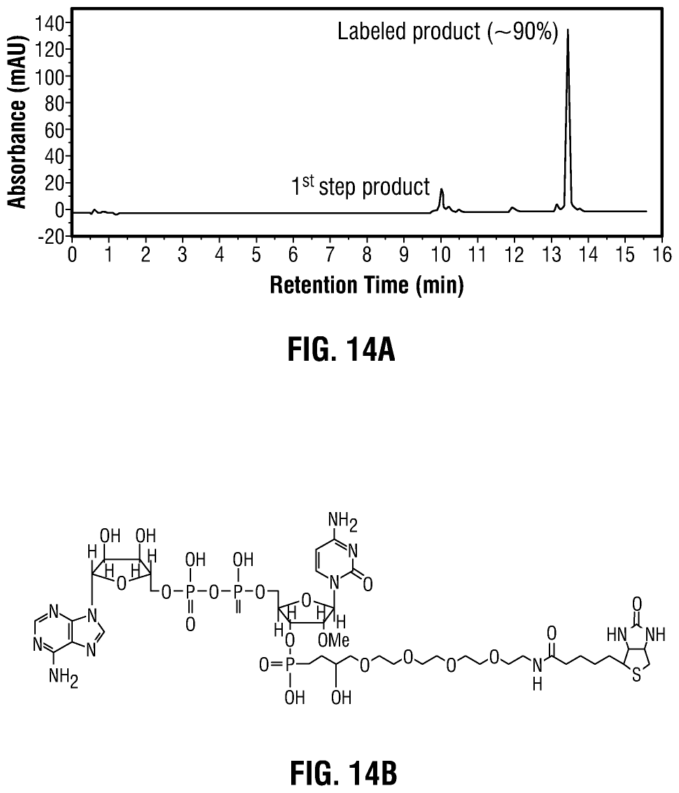

Enhancing RNA labeling efficiency. It remains a challenge to introduce tags, like biotin or fluorescent dyes, onto RNA with high yield. However, labeling two ends of RNA with selected tags is aa step of the direct RNA sequencing method disclosed herein. The labeling efficiency is directly related to how much of an RNA sample can be used to generate MS signals, with a higher labeling efficiency leading to a reduced sample requirement. To increase the labeling efficiency, new labeling strategies have continued to be optimize. A high labeling efficiency (˜90%) was recently observed when labeling the 5′ end of RNA with the 2-step reaction (FIG. 14A). The optimized reaction conditions include (i) replacing Cy3 with sulfo-Cy3 to increase aqueous solubility, (ii) adjusting the pH of the solution to 7.5, and (iii) lengthening the reaction time while maintaining constant stirring. While efforts to improve the labeling efficiency at the 5′ end of the RNA continue, it is expected to observe a similar high yield for 3′ end labeling following a published method (Cole K (2004) Nucleic Acids Res 32(11):e86-e86.1). To achieve this high efficiency, A(5′)pp(5′)Cp-TEG-biotin-3′ (FIG. 14B), an active form of biotinylated pCp, was chemically synthesize which will allow for the elimination of an adenylation step. Using such a strategy allows one to significantly improve the labeling efficiency to near quantitative yield at both ends.

Enhancing sequencing read length. In order to increase the read length, the molecular feature extraction (MFE) settings for Agilent MassHunter Qualitative Analysis were optimized. From the MFE data exported out of Agilent software, it was possible to automatically read longer RNAs up to 30 nt using the sequencing algorithm, a significant increase in read length compared to the ˜20-nt RNAs. It was also discovered that with the available software, there are two modes of identification depending on the size of the molecule: (i) a small molecule mode depending on accurate determination of the monoisotopic mass for identification, which works only to about 30-nt or ˜10,000 Da, judged by the RNA samples currently available; and (ii) a large molecule mode requiring accurate determination of the average mass for identification, which works only for molecules larger than about 30-nt.

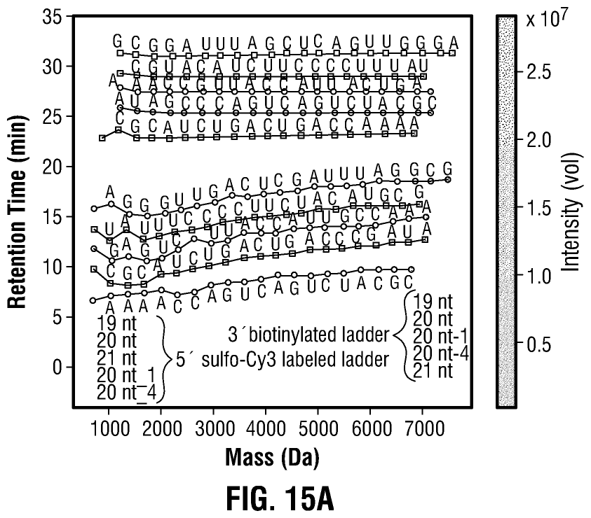

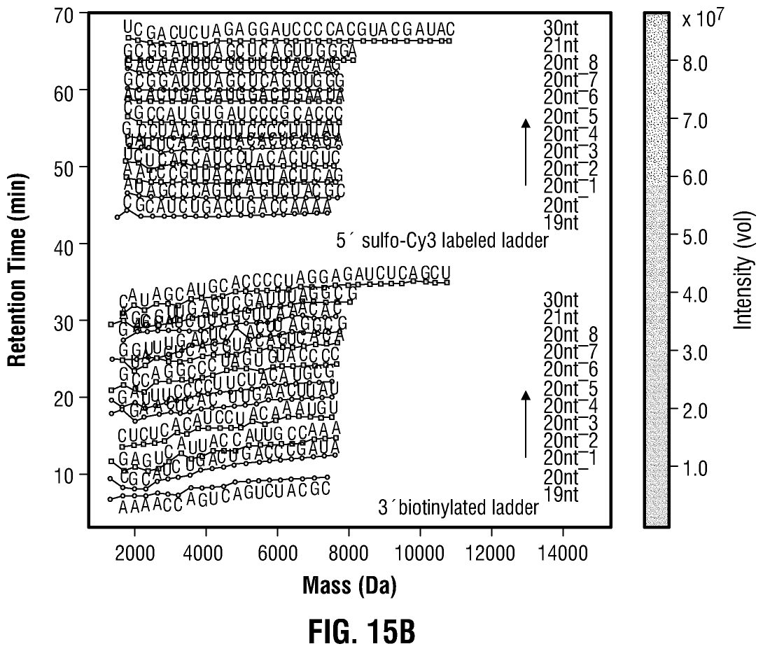

Enhancing sequencing throughput to multiple RNA strand sequencing of 5 and 12 RNAs. It has been demonstrated that the LC/MS-based method can not only sequence purified single stranded RNA, but also sequence RNA samples with multiple RNA strands. Two different RNAs could be read out, one 19 nt and one 20 nt simultaneously with the novel sample preparation protocol and bead separation described herein. A sample containing mixtures containing 5 and 12 RNAs has been tested. With the improvements in labeling efficiency and read length as described above, it was possible to detect all the ladder fragments needed for reading out the complete sequences of all the RNAs in these mixtures. This was achieved by (i) obtaining measurements on an Agilent 6550 ion-funnel Q-TOF LC/MS, and (ii) optimizing the MFE settings for Agilent MassHunter Qualitative Analysis. It was possible to manually read the sequences in the 5 and 12 RNA mixtures (FIG. 15A-B), including a 30 nt RNA (FIG. 15B). These results demonstrate that the direct RNA method described herein can sequence complex RNA samples with increased numbers of RNAs, leading to the requisite throughput needed to handle the various biological RNA samples.

### 8. Example

In order to increase the throughput and robustness of the MS-based sequencing method to enable sequencing of mixed RNA samples with multiple RNA strands, a new strategy was developed, as described herein, to optimize the experimental workflow and to significantly simplify 2D LC/MS data analysis for identifying the ladders needed for sequencing, while testing the efficacy of the new strategies on a series of synthetic RNA oligonucleotides of varying lengths containing both canonical and modified bases as a proof-of-concept study. It was possible to sequence pseudouridine (ψ) and 5-methylcytosine (m⁵C) simultaneously at single-base resolution. Together with the described end-labeling strategy, it was possible to identify, locate, and quantify these multiple base modifications while accurately sequencing the complete RNA not only in a single purified RNA strand, but also in sample mixtures containing 12 distinct sequences of RNAs.

### Results

### Generation of Labeled RNA Degraded Fragments for Mass Analysis

In the experimental approaches described herein, either one RNA end was labeled and the other end left unlabeled, or the two ends of the RNA were labeled with different tags to better distinguish them in the 2D LC/MS method. In one labeling strategy, a biotin tag was introduced to either the 3′ end or the 5′ end of the RNA prior to LC/MS analysis in order to introduce an RT and mass shift to exactly one mass ladder (14). This method can help simplify LC/MS data analysis and prevent confusion as to which fragment belongs to which ladder when sequencing mixed RNA samples. It increases the masses of RNA ladders so that the terminal bases can be identified, avoiding messy low mass regions where it is difficult to differentiate mononucleotides and dinucleotides from multi-cut internal fragments; improves sequencing accuracy by reading a complete sequence from one single ladder, rather than requiring paired-end reads; simplifies base-calling procedures, making it easier for the ladder components to be identified due to selective RT shifts; and improves sample efficiency by allowing for longer degradation time points (15 min) than reported before (5 min) (14). —These improvements can help reduce the minimum RNA sample loading requirement as compared to the first-generation method, increasing the potential to sequence endogenous RNA samples with rare RNA modifications.

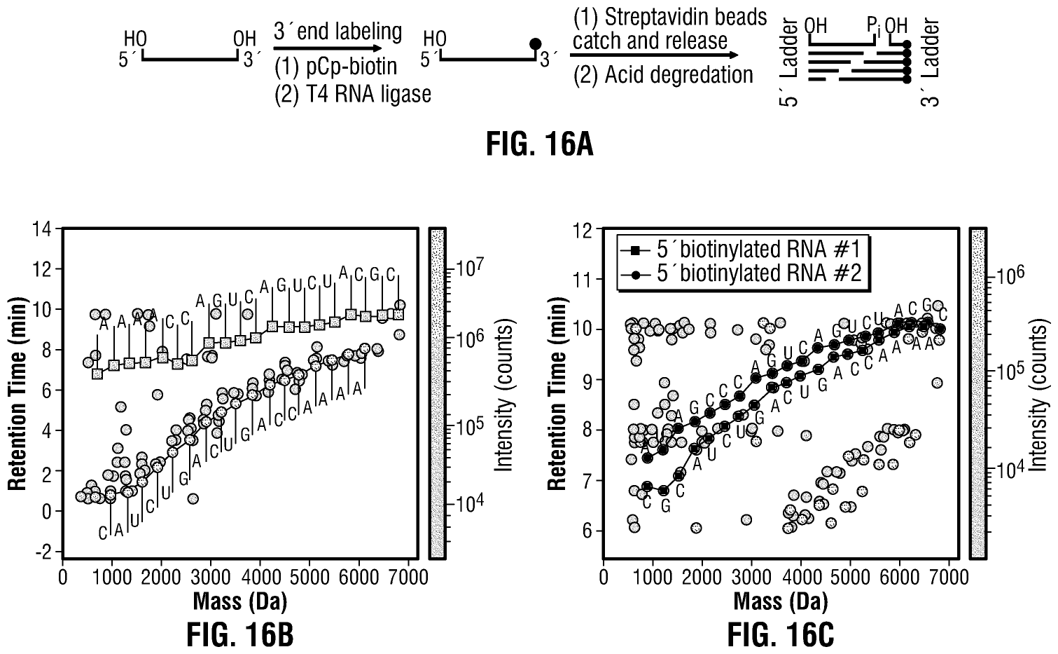

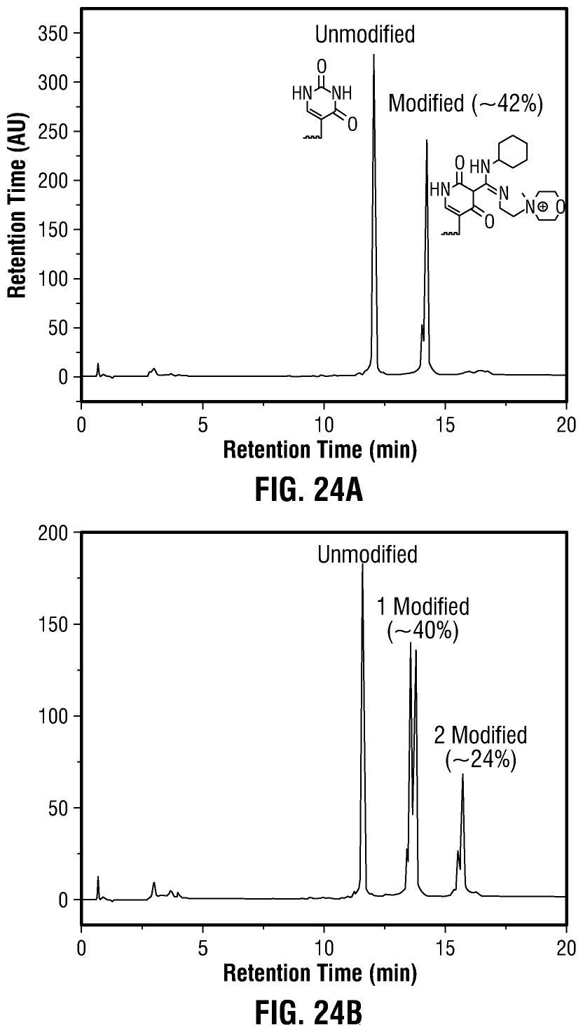

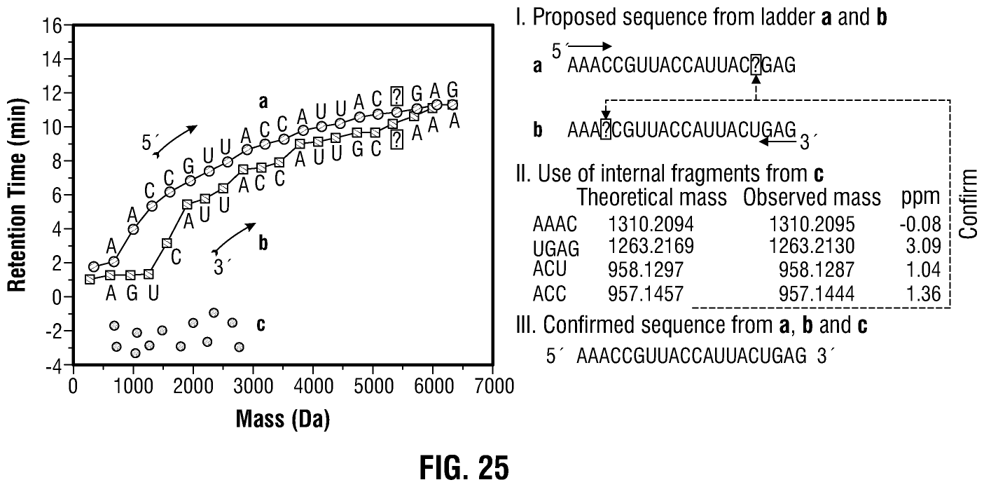

For labeling RNAs at their 3′ ends (FIG. 16A), biotinylated cytidine bisphosphate (pCp-biotin) was activated by adenylation using ATP and Mth RNA ligase to produce AppCp-biotin. Then, the members of the 3′ladder pool with a free 3′ terminal hydroxyl were ligated to the activated AppCp-biotin via T4 RNA ligase. Streptavidin-coupled beads were used to isolate the 3′-biotin-labeled RNA, which was released for acid degradation and subsequent LC/MS analysis after breaking the biotin-streptavidin interaction. This was also performed for 5′-end labeling as well (FIG. 24-25).

As a test example, short RNA oligonucleotides (19 nt and 20 nt RNA: RNA #1 and RNA #2, respectively) were designed and synthesized as model RNA oligonucleotides for individual and group tests. First, RNA #1 was 3′-biotin-labeled and subjected it to physical separation by streptavidin bead capture and release. In FIG. 16B, subsequent separation using RT shifts of a 3′-biotin-labeled mass ladder from an unlabeled 5′ ladder of RNA #1 avoids confusion as to which fragment belongs to which ladder, and the isolated curve in the output is much simpler to analyze than the two adjacent curves of the first-generation method. The de novo sequencing process was performed by a modified version of a published algorithm (14). This algorithm uses hierarchical clustering of mass adducts to augment compound intensity. Co-eluting neutral and charge-carrying adducts were recursively clustered, such that their integrated intensities were combined with that of the main peak. This increased the intensity of ladder fragment compounds, and reduced the data complexity in the regions critical for generating sequencing reads.

In FIG. 16B, the 3′ ladder curve is shifted up (with respect to the y-axis) because the biotin label causes an increase in RT, and the complete sequence of RNA #1 can be read from the top blue curve alone. Similarly, the complete RNA #1 reverse sequence can be read from the unlabeled 5′ladder curve (which does not have a shift in RT) directly, with the exception of the first nucleotide. Without this strategy, end pairing is required to read out the complete sequence, as reported before (14). With this advance, each RNA can be read out completely from one curve, and it is possible to sequence mixed samples containing multiple RNAs each labeled with a 5′biotin label (FIG. 16C). The separation of the 3′ and 5′ladders for each sample significantly reduces the complexity of the resultant LC/MS data so that it is much easier than the previous method (14) to find complete sets of ladder components needed for sequencing, and thus reduce the complexity of the base-calling procedures.

Because of this end labeling, both complete sequences in a mixture of two RNAs, one 19 nt (RNA #1) and one 20 nt (RNA #2) can be read out, from exactly one curve per RNA strand. In the case of this sample, the algorithm was used to perform crucial mass adduct clustering in order to further simplify the data for finding the complete sets of mass ladder components needed for sequencing. From the sigmoidal curves consisting of all the mass ladder components in the simplified 2D mass-RT plot (FIG. 16C), the sequences of the sample RNA strands can be manually determined (FIG. 16D) simply by calculating the mass differences of two adjacent ladder components. Although the samples are all synthetic samples and it was not necessary to use biotin-streptavidin binding-cleavage to physically separate the sample of interest from other RNA strands (one only actually required the RT shift associated with biotin-labeling), incorporation of the biotin label also provides the possibility of physical separation of specific samples that could be useful for sequencing real biological samples.

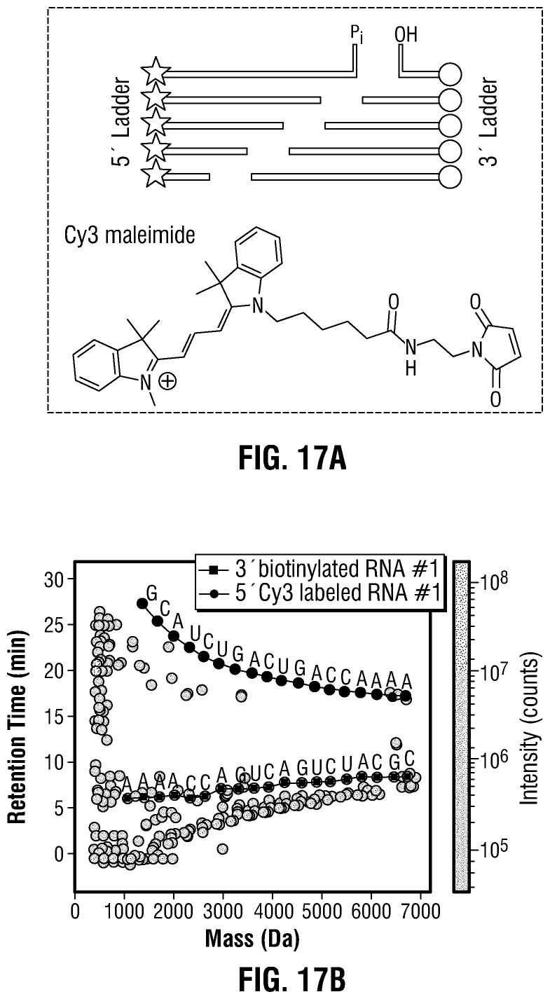

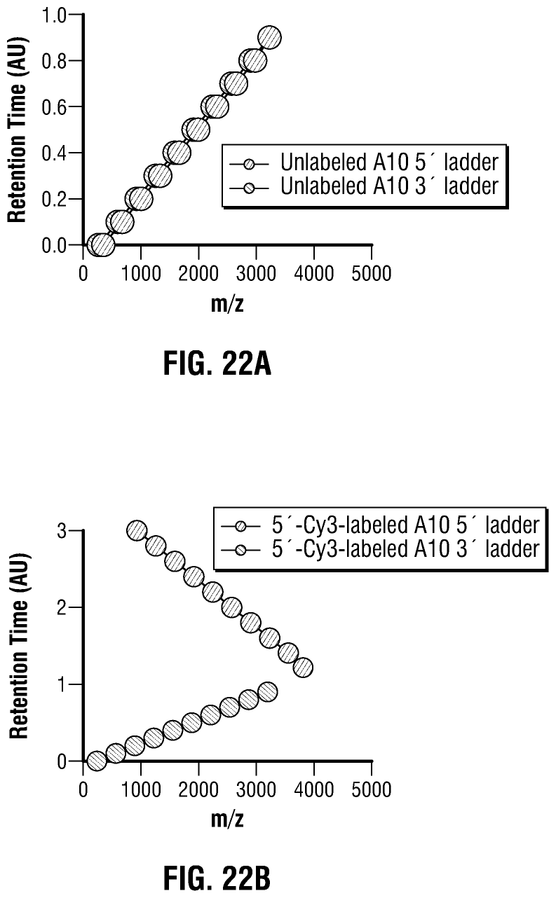

In order to further increase the observed RT shift afforded by end-labeling, an RNA sample may be labeled with other bulky moieties such as a hydrophobic cyanine 3 (Cy3) or cyanine 5 (Cy5). to magnify their RT difference. Different tags were introduced, such as Cy3, which is bulky and can cause a greater RT shift than biotin (14), at the 5′ end of the original RNA strand to be sequenced; a biotin moiety was introduced to the 3′ end of the RNA as described before. These end labels should systematically affect the RT of all 5′ and 3′ ladder fragments so as to differentiate the two ladder curves for sequencing, which was confirmed by in silico studies (FIGS. 22A and 22B). As shown in FIG. 17A, a Cy3 tag was added via a two-step reaction at the 5′end of the RNA sample. Similar to the 5′-biotinylation methodology, after thiolphosphorylation at the first step, Cy3 maleimide was conjugated to RNA. After acid degradation of the double end-labeled RNAs, the resulting fragments were directly subjected to LC/MS without any affinity-based physical separation. The preliminary data showed that in the mass-RT 2-D graph, the 5′ Cy3-labeled ladder fragments form a curve further away from the 5′ biotin-labeled ladder (FIG. 17B) as more hydrophobic tags elicit larger RT shifts. In fact, the RT trend for the Cy3-labeled 5′ ladder changes direction, as in the mass-RT plot, the sequence curve goes down in RT with increasing mass due to the hydrophobic nature of the Cy3 moiety, as compared to the biotin-labeled 3′ ladder, which goes up in RT with increasing mass (as also observed in all previous biotin-labeled and unmodified mass ladder samples). This results in two curves that are more separable/distinguishable during the 2-D analysis, making it easier to base call the sequences of the ladders even without physical separation. With bidirectional sequencing, the method's read length can be doubled, and its accuracy can be improved significantly by reading a complete sequence from both the 3′ and 5′ladders.

### RNA Labeling Efficiency

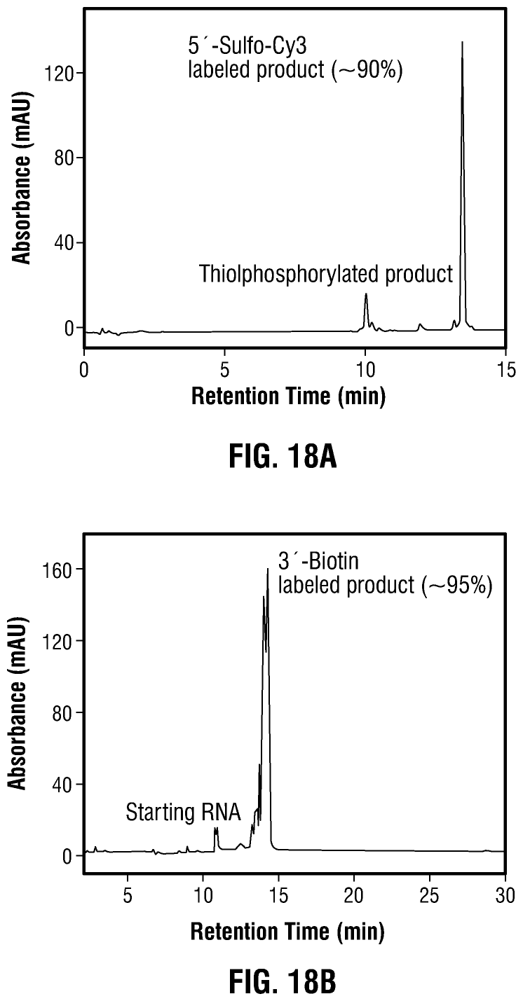

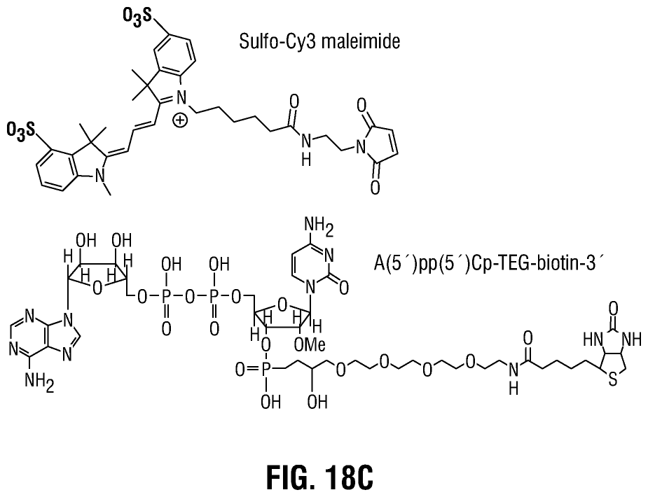

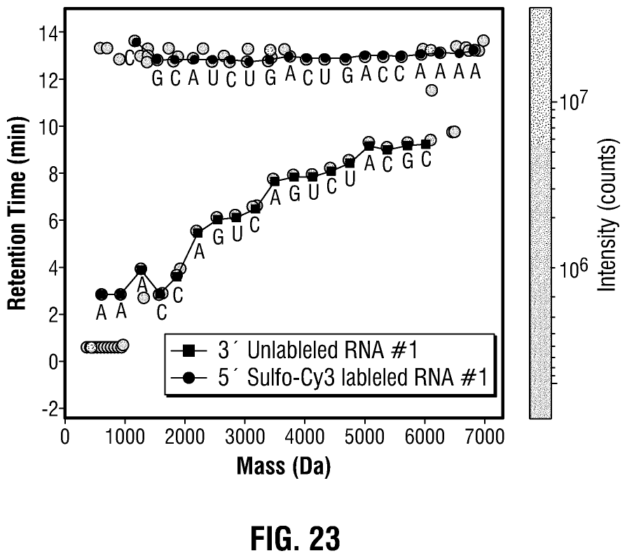

Despite various reported RNA labeling methods, it remains a challenge to introduce tags, like biotin or fluorescent dyes, onto RNA with high yield. However, labeling two ends of RNA with selected tags is a step of the direct RNA sequencing method disclosed herein. The labeling efficiency directly results in how much RNA sample can be used to generate MS signals, with a higher labeling efficiency leading to a reduced sample requirement. To increase the labeling efficiency, new labeling strategies have been explored and high labeling efficiency has been demonstrated at both the 5′ and 3′end (FIG. 18A). For the 5′end label, the labeling efficiency of full length RNA was improved from ˜60% (FIG. 17B) to ˜90% (FIG. 18A) by using a modified reaction protocol, including 1) using sulfo-Cy3 (FIG. 18C) instead of Cy3 to increase aqueous solubility of the tag, 2) adjusting the pH of the solution to 7.5, and 3) lengthening the reaction time while maintaining constant stirring. Even after acid degradation of a sulfo-Cy3 labeled RNA #1 it can be seen that the labeled ladder components far outnumber the unlabeled ladder components with respect to absolute intensity, as the unlabeled fragments do not appear on the plot after mild filtering (FIG. 23). For better labeling efficiency at the 3′ end, A(5′)pp(5′)Cp-TEG-biotin-3′ (FIG. 18C) was synthesized, an active form of biotinylated pCp, which eliminates the adenylation step (15). A highly yield (˜95%) for 3′ end labeling was observed (FIG. 18B) when labeling a 21 nt RNA (RNA #11) using this method. By incorporating both optimized end-labeling strategies into the sample preparation protocol, the minimum sample loading amount requirement is now less of a hindrance to the overall sequencing workflow.

### LC/MS Sequencing of Pseudourdine (ψ)

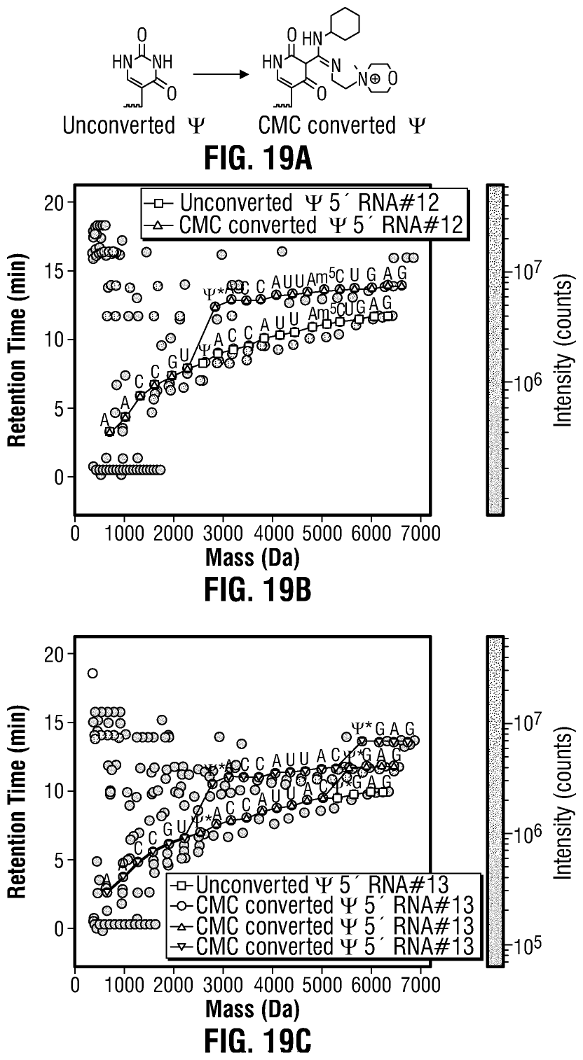

The new end labeling-LC/MS sequencing strategy was then applied to a synthetic sample containing a modified nucleobase. Pseudouridine (ψ) is the most abundant and widespread of all modified nucleotides found in RNA. It is present in all species and in many different types of RNAs, including both coding RNAs (mRNAs) and non-coding RNAs (16). However, it is impossible to distinguish w from U directly by MS because they have identical masses. An established chemical labeling approach was previously developed to distinguish ψ from U, relying on a nucleophilic addition with N-cyclohexyl-N′-(2-morpholinoethyl)-carbodiimide metho-p-toluenesulfonate (CMC) to form a CMC-ψ adduct (17). The CMC-ψ adduct stalls reverse transcription and terminates the cDNA one nucleotide towards the 3′ end downstream to it and is currently used to detect w sites in various RNAs at single-base resolution (18). Here, the same chemistry is adapted to form the same CMC-ψ adduct in our system (FIG. 19A). The adduct will not only have a unique mass 252.2076 Dalton larger than U's mass, but it is also more hydrophobic than the U, also resulting in an RT shift. The CMC-w adduct will thus significantly shift both the masses and RT of all the ladder fragments containing the CMC-ψ adduct in the mass-RT plot, which will help in identifying and locating the w in any of the RNA strands.

FIG. 24A and FIG. 24B show the HPLC profiles of the crude products of converting ψ to its CMC adducts in two RNAs using the reported conditions (18). These two RNAs contain 1 ψ and 2ψ moieties, respectively (RNA #12 and #13). The conversion percentage of ψ calculated by integrating peaks from UV chromatogram was ˜42% and ˜64%, respectively. For the RNA strand containing 2ψ nucleotides, their CMC conversion could be complete (both w nucleotides were converted to ψ-CMC adducts) or partial (only one of the 2ψ nucleotides was converted). Therefore, in FIG. 24B, the peak around 16 min refers to the RNA strand with complete conversion (˜24%), and the two adjacent peaks around 14 min reflect the partial conversion of either ψ (total ˜40%).

Automated sequencing was applied to RNA #12 and #13 after acid degradation by formic acid. In the 2D mass-RT plot (FIG. 19B) representing sequencing of a single w-containing RNA (RNA #12), a new curve (red) branched up off of the original sigmoidal curve (grey) at the ψ, corresponding to the part of the sequence with all CMC-ψ adduct-containing ladder fragments, which shift up and to the right in the 2-D mass-RT plot because the fragments with CMC-ψ adduct have 252.2076 Dalton larger masses and larger RTs than their corresponding unreacted ones. FIG. 19C depicts the 2D mass-RT plot representing sequencing of a double ψ-containing RNA (RNA #13). Similarly, one new curve (red) branched off at the second ψ, corresponding to the part of the sequence with conversion of both w to their CMC-ψ adducts. For ease of visualization, only the sequence of 5′mass-RT ladders are presented. Two additional curves (purple and orange) branched up off of the original unconverted 5′ ladder (grey curve) separately in each of two positions of the w nucleotides, indicating that the only one of two w nucleotides was converted. As such, it is possible to not only identify, locate, and quantify the base modification ψ in the ψ-containing RNA while reading out its complete sequence, but with further calculations incorporating the mass ladder intensity profiles, it is possible to also directly quantify the percentage of the CMC-containing RNA vs non-CMC-containing RNA in a given sample. Applying this strategy to other sequences, this method can allow one to accurately determine the percentage of RNA with any mass-altered modification vs its corresponding non-modified counterpart. Extending this idea to ψ, this method can allow one to estimate the percentage of ψ-containing RNA vs non-ψ-containing RNA if one can factor in the yield of CMC chemistry with ψ.

Sequencing an RNA Mixture with Multiple Modifications

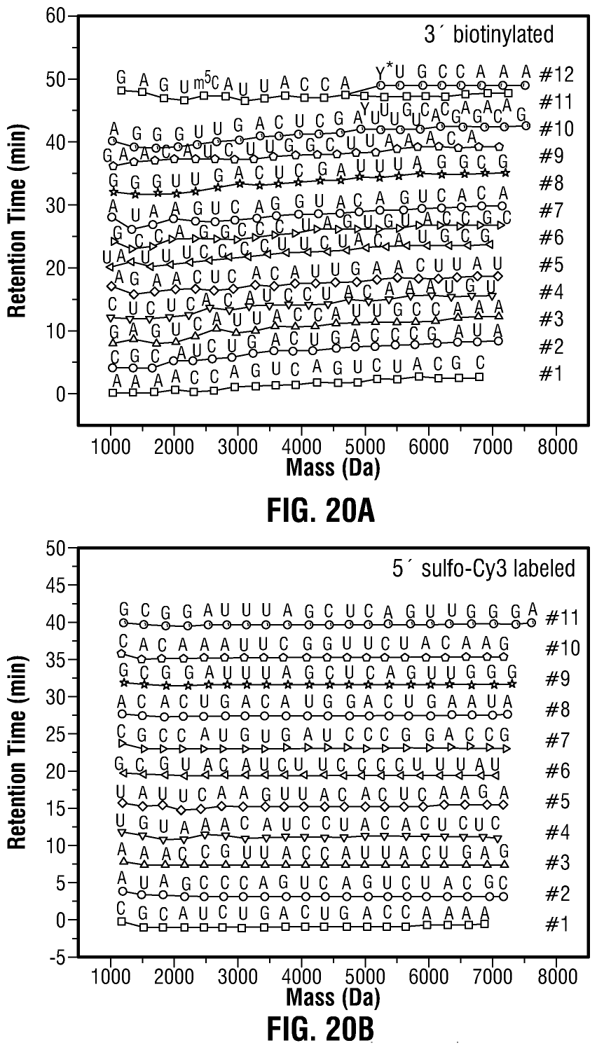

Finally, with the end-labeling and w base-modification methods in hand, it was next sought to increase the throughput of the method in order to sequence a multiplex RNA sample (simultaneous sequencing of a mixed sample containing multiple distinct RNA sequences) containing RNA strands with multiple modifications. A sample mixture containing 12 RNAs with distinct sequences, containing 11 unmodified RNAs and one multiply-modified RNA containing 1 ψ and 1 m⁵C, was subjected to the protocol. First, the 3′ ends of all RNA samples were chemically labeled with biotin, while sulfo-Cy3 was added to the 5′ ends (except for the RNA strand containing the base modifications). After measurement by LC/MS, the data were analyzed using Agilent MassHunter Qualitative Analysis software with optimized MFE settings to extract data for sequence generation. With the improvements in labeling efficiency described above, it was possible to detect all ladder fragments needed to accurately read out the complete sequences of all RNAs in the mixture. In the analysis of the multiplexed samples, the typical basecalling algorithm (as was used in all previous figures) was not used. These sequences were base-called manually, and all sequences could be read-out (FIGS. 20A AND 20B). The results showed that it was not only possible to sequence the four canonical nucleosides (A, C, G and U), but also again identify, locate, and quantify multiple modified bases at single-base resolution, such as w and m⁵C, or any other modified base, by mapping their masses in both single-stranded and mixed RNA samples. Similarly, for sequencing ψ, RNA was treated with CMC as described before, thus a new curve branched off of its corresponding non-CMC-containing ladder curve at the ψ (pink color). Although in these studies the sequences were manually read, as opposed to using an automated basecalling application, these studies show that there are no experimental or physical limitations in the sample preparation and mass spectrometry aspects of the system; the mass ladders of each component of the mixture can be properly generated, and can be accurately sequenced and basecalled by the mass-RT plot generated by the MFE file extracted from the LC/MS. These results show that the direct RNA method described herein can sequence more complex RNA samples with multiple RNAs containing modified bases, not just limited to purified single-stranded RNA containing one noncanonical bases as previously published (14). It is a significant step forward for MS sequencing of various complex biological RNA samples.

### Increase Sample Usage Via Utilization of Internal Fragments

Previous MS-based RNA sequencing methods controlled degradation conditions to generate well-defined mass ladders with single cuts for sequencing, as opposed to the unwanted appearance of multiple-cut fragments (14). As such, a 5 min formic acid treatment was performed to digest ˜10% of a 20 nt (RNA #3) sample into its corresponding 5′- and 3′-sequencing ladders to minimize formation of internal RNA fragments with more than one cut. (14) Thus, ˜90% of the starting material remained intact, and could not yield any sequence information. For real biological samples with low abundance, the fact that ˜90% of the sample would be unusable for sequencing results in the method's inability to generate enough signals to accurately sequence these low-abundance samples. In order to increase the percentage of usable sample, a longer degradation step is required. However, the process of generating more of the desired ladder fragments in a longer chemical/enzymatic degradation step will lead to the production of large amounts of internal fragments that do not possess a 5′ or 3′ end from the original RNA sequence by virtue of more than one cut-site on a given sequence (this is a stochastically-controlled process). The previous method (14) disregarded internal fragments simply as “noise” as they were not a part of the RNA ladders that were actually used in determining the sequence of bases and modification analysis. Although there is still inherent information in these internal fragments, utilizing information from internal fragments effectively is difficult because these sequences are mixed with the desired ladder compounds, especially for fragments in the lower mass regions with mass less than 2000 Daltons (Da). In this low mass region, monomer, dimer, and trimer nucleotides from any part of a given RNA strand cannot be easily separated in the LC phase of the LC/MS, leading to difficulty in accurate sequence identification and analysis. However, separation of desired ladder fragments from internal fragments by double-end labeling of the original sample before acid degradation makes it possible to actually take advantage of the previously unused internal fragments. It is proposed to gather and apply information from the internal fragments with more than one cut towards sequence generation/alignment where there are gaps (ironically generated from the same long acidic degradation step that generated the internal fragments) in the reported sequence greater than one missing base as observed in the sequence curve of the 2-D mass-RT plot of an RNA sample which has been subjected to a 60 min degradation step. As shown in FIG. 25, by combining three pieces of information: (a) the 5′ladder, (b) the 3′ladder, and (c) internal fragments without both ends, the RNA sequencing accuracy can be significantly increased as gaps (unassignable bases) in the mass-RT ladder caused by long degradation times can potentially be completely removed.

Development of 2D-mass-RT direct RNA sequencing methodology brings the power of MS-based laddering technology to RNA, addressing a long-standing unmet need in the broad field of RNA modification studies. Not only does it provide a direct method for RNA sequencing without the need of a cDNA intermediate, it also provides a general method for sequencing multiple base modifications on multiple RNA strands in one single experiment. The developed method has been proven successful to sequence short single strands of synthetic RNA (˜20 nucleotides) (FIG. 17). With end-labeling, it is no longer require to pair end sequencing for the complete sequence coverage as before; as it is possible to read out the complete sequence of a given RNA strand from either the 3′ or the 5′end, thus increasing the throughput and ease of data analysis. By using end-labeling, it is possible to extend the method to directly sequence multiplexed RNA mixtures (FIG. 20), which is a crucial step forward in MS-based sequencing of cellular RNA samples, typically consisting of mixed RNAs of unknown sequence. Additionally, the power of the method in sequencing multiple modified bases in this work, including pseudouridine and m⁵C, allowing one to identify, locate, and quantify each of these RNA modifications at single base resolution in the mixed samples with 12 RNA strands.

Accordingly, the sequencing methods disclosed herein can facilitate the efficient sequencing of modified RNA molecules, including, for example, tRNAs, siRNAs, therapeutic synthetic oligoribonucleotides having pharmacological properties, mixtures of RNA molecules, as well as detection of modifications of such RNA molecules. This approach may be expanded to sequence cellular RNAs with known chemical modifications, such as endogenous tRNA and mRNA, to benchmark the method's efficacy in read length and identification of extensive modifications. It is expected that this direct MS-based RNA sequencing method will facilitate the discovery of more unknown modifications along with their location and abundance information, which no other established sequencing methods are currently capable of. With continued improvements in read length, this direct sequencing strategy can be expanded to sequence longer RNAs, such as mRNA and long non-coding RNA, and pinpoint the chemical identity and position of nucleotide modifications.

### Methods

### Chemical Materials

The following RNA oligonucleotides were obtained from Integrated DNA Technologies and used without further purification (Coralville, Iowa, USA).

RNA #1: 5′-HO-CGCAUCUGACUGACCAAAA-OH-3′
RNA #2: 5′-HO-AUAGCCCAGUCAGUCUACGC-OH-3′
RNA #3: 5′-HO-AAACCGUUACCAUUACUGAG-OH-3′
RNA #4: 5′-HO-UGUAAACAUCCUACACUCUC-OH-3′
RNA #5: 5′-HO-UAUUCAAGUUACACUCAAGA-OH-3′
RNA #6: 5′-HO-GCGUACAUCUUCCCCUUUAU-OH-3′
RNA #7: 5′-HO-CGCCAUGUGAUCCCGGACCG-OH-3′
RNA #8: 5′-HO-ACACUGACAUGGACUGAAUA-OH-3′
RNA #9: 5′-HO-GCGGAUUUAGCUCAGUUGGG-OH-3′
RNA #10: 5′-HO-CACAAAUUCGGUUCUACAAG-OH-3′
RNA #11: 5′-HO-GCGGAUUUAGCUCAGUUGGGA-OH-3′
RNA #12: 5′-HO-AAACCGUψACCAUUAm⁵CUGAG-OH-3′
RNA #13: 5′-HO-AAACCGUψACCAUUACψGAG-OH-3′

Formic acid (98-100%) was purchased from Merck (Darmstadt, Germany). Biotinylated cytidine bisphosphate (pCp-biotin), {Phos (H)}C{BioBB}, was obtained from TriLink BioTechnologies (San Diego, Calif., USA). Adenosine-5′-5′-diphosphate-{5′-(cytidine-2′-O-methyl-3′-phosphate-TEG}-biotin, A(5′)pp(5′)Cp-TEG-biotin-3′, was synthesized by ChemGenes (Wilmington, Mass., USA). T₄ DNA ligase 1, T₄ DNA ligase buffer (10×), the adenylation kit including reaction buffer (10×), 1 mM ATP, and Mth RNA ligase were obtained from New England Biolabs (Ipswich, Mass., USA). ATPγS and T4 polynucleotide kinase (3′-phosphatase free) were obtained from Sigma-Aldrich (St. Louis, Mo., USA). Biotin maleimide was purchased from Vector Laboratories (Burlingame, Calif., USA). Cyanine3 maleimide (Cy3) and sulfonated Cyanine3 maleimide (sulfo-Cy3) were obtained from Lumiprobe (Hunt Valley, Md., USA). The streptavidin magnetic beads were obtained from Thermo Fisher Scientific (Waltham, Mass., USA). Chemicals needed for conversion of pseudouridine including CMC (N-cyclohexyl-N′-(2-morpholinoethyl)-carbodiimide metho-p-toluenesulfonate), bicine, urea, EDTA and Na₂CO₃ buffer, were obtained from Sigma-Aldrich (St. Louis, Mo., USA).

### Workflow

(1) Chemical conversion of pseudouridine was applied for distinguishing pseudouridine from uridine. (2) Labels were added on one or both ends of RNA strands with optimized experimental procedures. (3) The single RNA strand or mixtures of RNA strands was/were degraded into a series of short, well-defined fragments (sequence ladder), ideally by random, sequence context-independent, and single-cut cleavage of phosphodiester bonds on each RNA strand over its entire length, through a 2′-OH-assisted acidic hydrolysis mechanism. (4) If needed, physical separation of biotinylated RNA from unlabeled RNA using streptavidin-coated magnetic beads. (5) The digested fragments were then subjected to LC/MS analysis and the deconvoluted masses and RTs were analyzed to identify each ladder fragment. (6) Algorithms were applied to automate the data processing and sequence generation process.

### 3′ End Labeling Method

Use a two-step protocol. (1) Adenylation: The following reaction was set up with a total reaction volume of 10 μL in an RNAse-free, thin walled 0.5 mL PCR tube: 1× adenylation reaction buffer (5′ adenylation kit), 100 μM of ATP, 5.0 μM of Mth RNA ligase, 10.0 μM pCp-biotin, and nuclease-free, deionized water (Thermo Fisher Scientific, USA). The reaction was incubated in a GeneAmp™ PCR System 9700 (Thermo Fisher Scientific, USA) at 65° C. for 1 hour followed by the inactivation of the enzyme Mth RNA ligase at 85° C. for 5 minutes. (2) Ligation: A 30 μL reaction solution contained 10 μL of reaction solution from the adenylation step, 1× reaction buffer, 5 μM target RNA sample, 10% (v/v) DMSO (anhydrous dimethyl sulfoxide, 99.9%, Sigma-Aldrich, USA), T4 RNA ligase (10 units), and nuclease-free, deionized water. The reaction was incubated for overnight at 16° C., followed by column purification.

For the one-step protocol. A(5′)pp(5′)Cp-TEG-biotin-3′ was applied to improve the labeling efficiency by eliminating the adenylation step, while simplify the labeling method. The ligation step was achieved by a 30 μL reaction solution containing 1× reaction buffer, 5 μM target RNA sample, 10 μM A(5′)pp(5′)Cp-TEG-biotin-3′, 10% (v/v) DMSO, T4 RNA ligase (10 units), and nuclease-free, deionized water. The reaction was incubated for overnight at 16° C., followed by column purification. Oligo Clean & Concentrator (Zymo Research, Irvine, Calif., U.S.A.) was used to remove enzymes, free biotin, and short oligonucleotides.

### 5′ End Labeling Method

Biotin labeling at the 5′end required two steps. In an RNase-free, thin walled PCR tube (0.5 mL) containing 10× reaction buffer, 90 μM of RNA, 1 mM of ATPγS, and 10 units of T4 polynucleotide kinase, bringing the total reaction volume to 10 μL with nuclease-free, deionized water, incubation was carried out for 30 minutes at 37° C. Then 5 μL of biotin maleimide that was dissolved in 312 μL anhydrous DMF (anhydrous dimethyl sulfoxide, 99.9%, Sigma-Aldrich, USA) was added, mixed by vortexing, and incubated the sample for 30 minutes at 65° C. Column purification using Oligo Clean & Concentrator was performed as described above.

A different tag, such as a hydrophobic Cy3 (cyanine 3) or Cy5 (cyanine 5) tag, was introduced to the 5′end by the same method as above (except through Cy3-maleimide or sulfo-Cy3 maleimide replacement of the biotin maleimide), to distinguish its ladder from the 3′ biotinylated ladder. The optimization of the reaction conditions, compared to the above described 2-step protocol, was performed to obtain high labeling efficiency in the following manner: 1) sulfo-Cy3 was used for obtaining high water solubility with a molar ratio of reactants at 50:1 (sulfo-Cy3 to RNA); 2) the pH of the reaction solution was adjusted to 7.5 by Tris-HCl buffer (1 M) with a final concentration of 50 mM; and 3) the reaction time was lengthened to overnight (16 hrs) with constant stirring.

### Acid Hydrolysis Degradation

Unless otherwise indicated, formic acid was applied to degrade full length RNA samples for producing mass ladders.^{30,31} Each RNA sample solution was divided into three equal aliquots for formic acid degradation using 50% (v/v) formic acid at 40° C., with one reaction running for 2 min, one for 5 min, and one for 15 min. For the experiments regarding generation of internal fragments (FIG. S4), a 60 min formic acid treatment was performed on RNA #3. The reaction mixture was immediately frozen on dry ice followed by lyophilization to dryness, which was typically completed within 30 minutes. The dried samples were combined and suspended in 20 μL nuclease-free, deionized water for the subsequent biotin/streptavidin capture/release step or stored at −20° C. for LC/MS measurement. In FIG. 20, the experiment was started with two separate samples of the same 11 sequences (RNA #1-RNA #11), one with a 3′-biotin-label and one with a 5′-sulfo-Cy3 label, and mixed these samples along with a sample containing 3′-biotin-labeled RNA #12 before injection into the LC/MS.

### Biotin/Streptavidin Capture/Release Step

Biotin/Streptavidin capture uses streptavidin-coated magnetic beads to bind biotin-labeled RNAs, which are selectively immobilized onto streptavidin-coated magnetic beads and drawn to a magnet. Bound RNAs should, therefore, be isolated from non-biotin labeled RNAs and impurities (which remain in solution and will be washed away) and can be later eluted from the beads for LC-MS sequencing analysis. For the sample in FIG. 16B (no other samples required this step), 200 μL of Dynabeads™ MyOne™ Streptavidin Cl beads were prepared by first adding an equal volume of 1× B&W buffer. This solution was vortexed and placed on the magnet for 2 min, followed by discarding of the supernatant. The beads were washed twice with 200 μL of Solution A (DEPC-treated 0.1 M NaOH and DEPC-treated 0.05 M NaCl) and once in Solution B (DEPC-treated 0.1 M NaCl). A final addition of 100 μL of 2×B&W buffer brought the concentration of the beads to 20 mg/mL. An equal volume of biotinylated RNA in 1× B&W buffer was then added, and the sample was incubated for 15 min at room temperature using gentle rotation, followed by placing the tube on the magnet for 2 min, and discarding the supernatant. The coated beads were washed 3 times in 1× B&W buffer and the final concentration of each wash step supernatant was measured by Nanodrop for recovery analysis, to confirm that the target RNA molecules remained on the beads. For releasing the immobilized biotinylated RNAs, the beads were incubated in 10 mM EDTA (Thermo Fisher Scientific, USA), pH 8.2 with 95% formamide (Thermo Fisher Scientific, Waltham, Mass., USA) at 65° C. for 5 min. Finally, this sample tube was placed on the magnet for 2 min and the supernatant (containing the target RNA molecules) was collected by pipet.

Chemistry for Differentiating Pseudouridine from Uridine

The experimental approach to modify pseudouridine was performed according to the report by Bakin and Ofengand (Bakin, A.; Ofengand, J. Biochemistry 1993, 32 (37), 9754-62). Each RNA sample (1 nmol) was treated with 0.17 M CMC in 50 mM Bicine, pH 8.3, 4 mM EDTA, and 7 M urea at 37° C. for 20 min in a total reaction volume of 90 μL. The reaction was stopped with 60 μL of 1.5 M NaOAc and 0.5 mM EDTA, pH 5.6 (buffer A). After purification using an Oligo Clean & Concentrator, 60 μL of 0.1 M Na₂CO₃ buffer, pH 10.4 was added into the solution, brought to a reaction volume of 120 μL, and incubated at 37° C. for 2 h. The reaction was stopped with buffer A and purified by Oligo Clean & Concentrator.

### LC-MS Analysis

Samples were separated and analyzed on a 6550 Q-TOF mass spectrometer coupled to a 1290 Infinity LC system equipped with a MicroAS autosampler and Surveyor MS Pump Plus HPLC system (Agilent Technologies, Santa Clara, Calif., USA) (Hunter Mass Spectrometry, NY, USA). All separations were performed reversed-phase HPLC using an aqueous mobile phase (A), 25 mM hexafluoro-2-propanol (HFIP) (Thermo Fisher Scientific, USA) with 10 mM diisopropylamine (DIPA) (Thermo Fisher Scientific, USA) at pH 9.0 and an organic mobile phase (B), methanol across a 50 mm×2.1 mm Xbridge C18 column with a particle size of 1.7 μm (Waters, Milford, Mass., USA). The flow rate was 0.3 mL/min, and all separations were performed with the column temperature maintained at 35° C. Injection volumes were 20 μL, and sample amounts were 15-400 pmol of RNA. Data were recorded in negative polarity. The sample data were acquired using the MassHunter Acquisition software (Agilent Technologies, USA). To extract relevant spectral and chromatographic information from the LC-MS experiments, the Molecular Feature Extraction workflow in MassHunter Qualitative Analysis (Agilent Technologies, USA) was used. This proprietary molecular feature extractor algorithm performs untargeted feature finding in the mass and retention time dimensions. In principal, any software capable of compound identification could be used. The software settings were varied depending on the amount of RNA used in the experiment. In general, the goal was to include as many identified compounds as possible, up to a maximum of 1000. For samples with low concentrations, profile spectral peaks were filtered using a signal-to-noise ratio (SNR) threshold of 5 and, for more concentrated samples, an SNR threshold of up to 20. The other algorithm settings were as follows: “Small Molecules (chromatographic)” extraction algorithm, charge states from −1 to −15, only loss of hydrogen (—H) ions, “Common Organic Molecules” isotope model, minimum quality score 70 (range 0-100), and minimum ion count 500.

In addition to automating the sequence generation, manually reading RNA sequences was also used to confirm the accuracy of the automating sequencing. These sequences were manually read out from the data extracted by the Molecular Feature Extraction (MFE) algorithm integrated in the Agilent's software of MassHunter Qualitative Analysis. In Tables S1-S38, provided are the theoretical mass of each fragment (obtained by ChemDraw), base mass, base name, observed mass, RT, volume (peak intensity), quality score, and ppm mass difference. All figures presented are representative data of multiple experimental trials (n≥3). For ease of visualization, the 5′-sulfo-Cy3 labeled mass ladders and the 3′-biotinylated mass ladders were plotted separately (i.e., 3′-biotinylated mass ladders were all plotted in FIG. 20A and the 5′-sulfo-Cy3 labeled mass ladders were all plotted in FIG. 20B). Then, for each sequence curve (up to 12 on a given plot), the starting RT values were normalized to start at 4 minute intervals (except in the case of RNA #12 in FIG. 20A, where an 8-minute interval gap was used). The absolute differences between the starting RT value and subsequent RT values of any single given curve remain unchanged; only the visual “height” at which each curve is plotted was changed. Plots for FIG. 20 were produced with OriginLab, a commercial picture-making software. In all figures except Fig. FIG. 20A-B, the mass-RT plot was generated without normalization of any of the RT values. Because of a missing base assignment in the original sample, two samples were combined and analyzed and visualized the combined data in FIG. 17B. One sample contained RNA #1 with both 5′-Cy3 and 3′-biotin labels, while the second combined sample contained RNA #1 with only a 5′-Cy3 label (Table S6).

### Automated RNA Sequencing and Visualization Algorithm

The first step of the LC/MS data analysis is to perform data pre-processing and reduction so that the LC/MS data will become less noisy, and consequently easier to read out the RNA sequence(s) from the data in the next step. From the multi-dimensional LC/MS data, there are several dimensions that can be used to pre-process the data and reduce its volume, such as Retention Time (RT), Intensity (Volume), and Quality Score (QS). Please see Supplementary Information for details on data processing and modifications to the sequencing algorithm. The source code of the revised algorithm is available. Further improvement of the algorithm will enable one to automate base-calling and modification identification when sequencing more complicated cellular RNAs.

### Quantifying Stoichiometry/Percentage of Modified RNA in a Partially Modified RNA Sample

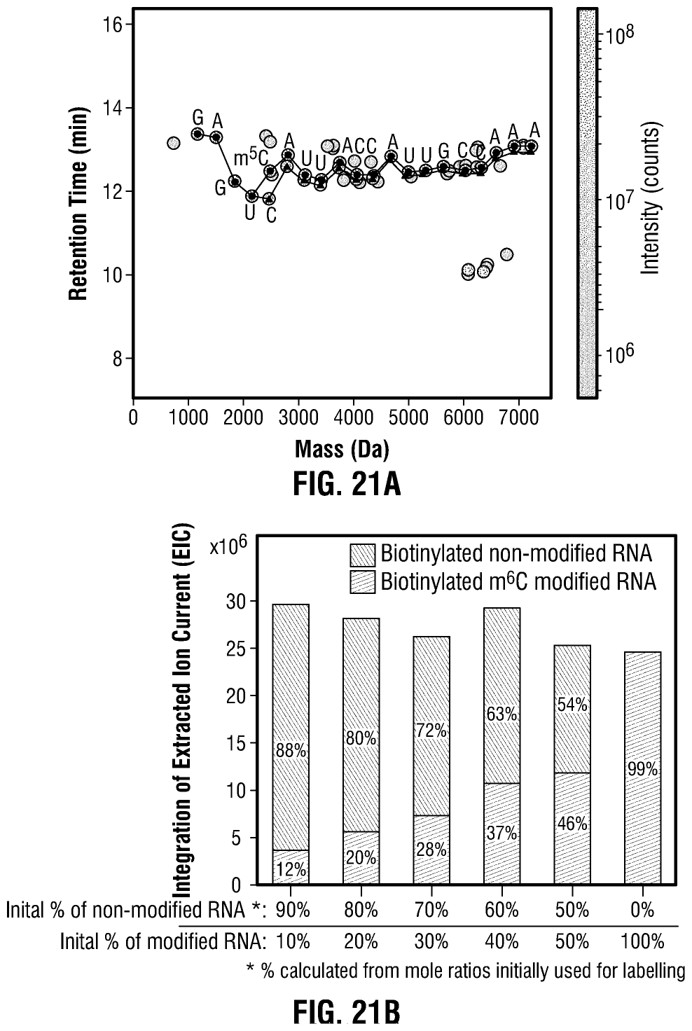

Understanding the dynamics of cellular RNA modifications (20, 21) requires a method to quantify the stoichiometry/percentage of RNA with site-specific modifications vs. its canonical counterpart RNA, as base modifications may not occur on 100% of all identical RNA sequences in a cell or sample. Applying the above quantification strategy to other sequences, this method is expected to allow one to accurately determine the percentage of RNA with any mass-altered modification vs. its corresponding non-modified counterpart. As shown in FIG. 21, not only can the complete sequence including the m5C be read out accurately from the mixture containing both modified and non-modified RNA (FIG. 21A), but the relative percentage of m5C modified RNA (20%) vs. its non-modified counterpart (80%) can also be quantified based upon information from the extracted ion chromatograph (FIG. 21B) (21). The relative quantities of different product species were quantified by integrating the extracted ion current (EIC) peaks of 3′-biotin labeled methylated RNA and non-modified RNA before their formic acid degradation. In addition to sequencing, RNA mixtures with other different ratios have also been quantified similarly (FIG. 21B). These relative percentages match well with the ratios of the absolute amounts of RNA initially used for RNA labeling with a difference less than 5%, indicating that EIC-based integration is an accurate method for relative quantification of modified RNA when not every RNA with the same sequence was modified. Extending this idea to ψ, this method can allow one to estimate the percentage of ψ-containing RNA vs. non-ψ-containing RNA if one can factor in the yield of CMC chemistry with ψ.

Adding a 5′ tag to spatially separate ladders on a retention time (RT) vs. mass plot, a simulated mass spectrum peak set for both 5′ and 3′ ladders of a synthetic, unmodified A10 (10-mer of polyadenine) sequence was first generated in silico. Each row represents a given mass ladder peak, and each peak was assigned a unitless retention time (RT) and an arbitrarily constant unitless peak volume of 1000. The RT assigned for each ladder increased systematically with increasing mass, starting with 0 and increasing in 0.1 unit increments. The peak list for the simulated A10 mass spectrum was as follows:

A10 - unmodified MS peak list

| Mass | t_{R} | Vol |
|---|---|---|
| 347.063065 | 0 | 1000 |
| 676.115565 | 0.1 | 1000 |
| 1005.168065 | 0.2 | 1000 |
| 1334.220565 | 0.3 | 1000 |
| 1663.273065 | 0.4 | 1000 |
| 1992.325565 | 0.5 | 1000 |
| 2321.378065 | 0.6 | 1000 |
| 2650.430565 | 0.7 | 1000 |
| 2979.483065 | 0.8 | 1000 |
| 3228.569232 | 0.9 | 1000 |
| 267.096732 | 0 | 1000 |
| 596.149232 | 0.1 | 1000 |
| 925.201732 | 0.2 | 1000 |
| 1254.254232 | 0.3 | 1000 |
| 1583.306732 | 0.4 | 1000 |
| 1912.359232 | 0.5 | 1000 |
| 2241.411732 | 0.6 | 1000 |
| 2570.464232 | 0.7 | 1000 |
| 2899.516732 | 0.8 | 1000 |
| 3228.569232 | 0.9 | 1000 |

The mass ladder starting from 347.063065 represents the 5′mass ladder, while the mass ladder starting from the 267.096732 represents the 3′mass ladder.

Next a simulated mass spectrum peak set for both 5′ and 3′ladders of a synthetic, 5′-cyanine 3 (Cy3)-labeled A10 (10-mer of polyadenine) sequence was generated in silico. This was done by taking the data set above, and adding the additional mass afforded by a 5′-Cy3 label (614.3061) to each member of the 5′-ladder in the data set. The peak volumes did not change. The associated RT for this new Cy3-labeled 5′-ladder was generated by now starting from an RT of 10, and decreased by an increment of 0.2 with increasing mass. This was done to simulate the potential change to an RT vs. mass spectrum of any end-labeled ladder (in this case, 5′-Cy3-labeled) in both absolute RT values, RT trends (monotonically increasing curve to a monotonically decreasing curve, for example), and absolute mass values. Of course, real changes in all of these values in a real system could not be absolutely predicted in silico, and thus this should be only taken as a proof-of-principle example. The peak list for the simulated 5′-Cy3-labeled A10 mass spectrum was as follows:

A10 - 5′-Cy3-labeled MS peak list

| Mass | t_{R} | Vol |
|---|---|---|
| 961.369165 | 3 | 1000 |
| 1290.421665 | 2.8 | 1000 |
| 1619.474165 | 2.6 | 1000 |
| 1948.526665 | 2.4 | 1000 |
| 2277.579165 | 2.2 | 1000 |
| 2606.631665 | 2 | 1000 |
| 2935.684165 | 1.8 | 1000 |
| 3264.736665 | 1.6 | 1000 |
| 3593.789165 | 1.4 | 1000 |
| 3842.875332 | 1.2 | 1000 |
| 267.096732 | 0 | 1000 |
| 596.149232 | 0.1 | 1000 |
| 925.201732 | 0.2 | 1000 |
| 1254.254232 | 0.3 | 1000 |
| 1583.306732 | 0.4 | 1000 |
| 1912.359232 | 0.5 | 1000 |
| 2241.411732 | 0.6 | 1000 |
| 2570.464232 | 0.7 | 1000 |
| 2899.516732 | 0.8 | 1000 |
| 3228.569232 | 0.9 | 1000 |

The mass ladder starting from 961.369165 represents the 5′-Cy3-labeled mass ladder, while the mass ladder starting from the 267.096732 represents the 3′mass ladder.

Comparing these two RT vs. mass plots, one sees that the two mass ladder curves are almost superimposed when there is no end-labeling (FIG. 22A), resulting in potential mis-sequencing in downstream basecalling and sequence identification, while the 5′-Cy3-labeled sample has two distinct and separate mass ladder curves (FIG. 22B), which allow for greater ease of visualization of all the ladder components needed for sequencing and higher accuracy in downstream basecalling and sequence identification.

In addition to automating the sequence generation, one can also manually search for the mass ladders by the Molecular Feature Extraction (MFE) workflow in MassHunter Qualitative Analysis (Agilent Technologies), for confirming the accuracy of automating sequencing. In Table S1-S38, provided are the theoretical mass of each fragment (obtained by ChemDraw), base mass, base name, observed mass, RT, volume (peak intensity), quality score, and error expressed as ppm (calculated by the equation as follows). The MFE settings were optimized to extract as many identified compounds as possible but with reasonable quality score. The MFE settings applied are as follows: “centroid data format, small molecules (chromatographic), peak with height≥500, quality score≥70”. However, data reduction was performed to simplify algorithm sequencing if needed. For instance, retention time could be selected from 6 to 10 min for biotin labeled samples for a 20 nt RNA. Also, the numbers of input compounds used for algorithm analysis are generally an order-of-magnitude higher than the numbers ladder fragments needed for generating complete sequences, unless indicated otherwise; these input compounds are sorted out of all MFE extracted compounds typically with higher volumes and/or better quality scores.

The following formula was used to calculate the PPM described in Example 8:

ppm=10⁻⁶×(Mass_{theoretical}−Mass_{observed})/Mass_{theoretical}

TABLE S1
LC/MS analysis of 3′biotin-labeled RNA #1 after isolation by streptavidin beads
followed by subsequent chemical degradation (3′labeled mass ladder components, RNA #1).

| heoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6781.0733 | 305.0413 | C | 6781.0413 | 9.752 | 16819442 | 100 | 4.72 |
| 18 | 6476.0320 | 345.0474 | G | 6475.9924 | 9.717 | 247965 | 84 | 6.11 |
| 17 | 6130.9846 | 305.0413 | C | 6130.9398 | 9.662 | 178841 | 80 | 7.31 |
| 16 | 5825.9433 | 329.0525 | A | 5825.9037 | 9.782 | 510096 | 80 | 6.80 |
| 15 | 5496.8908 | 306.0253 | U | 5496.8566 | 9.383 | 262486 | 99 | 6.22 |
| 14 | 5190.8655 | 305.0413 | C | 5190.8364 | 9.241 | 349988 | 100 | 5.61 |
| 13 | 4885.8242 | 306.0253 | U | 4885.7908 | 9.135 | 356118 | 100 | 6.84 |
| 12 | 4579.7989 | 345.0475 | G | 4579.7738 | 9.109 | 386687 | 100 | 5.48 |
| 11 | 4234.7514 | 329.0525 | A | 4234.7271 | 9.145 | 305380 | 100 | 5.74 |
| 10 | 3905.6989 | 305.0413 | C | 3905.6749 | 8.575 | 145505 | 96 | 6.14 |
| 9 | 3600.6576 | 306.0253 | U | 3600.6373 | 8.420 | 195308 | 100 | 5.64 |
| 8 | 3294.6323 | 345.0474 | G | 3294.6165 | 8.370 | 125991 | 100 | 4.80 |
| 7 | 2949.5849 | 329.0525 | A | 2949.5716 | 8.339 | 106993 | 100 | 4.51 |
| 6 | 2620.5324 | 305.0413 | C | 2620.5193 | 7.492 | 90629 | 100 | 5.00 |
| 5 | 2315.4911 | 305.0413 | C | 2315.4814 | 7.299 | 163692 | 100 | 4.19 |
| 4 | 2010.4498 | 329.0525 | A | 2010.4388 | 7.625 | 279963 | 100 | 5.47 |
| 3 | 1681.3973 | 329.0525 | A | 1681.3891 | 7.354 | 183827 | 100 | 4.88 |
| 2 | 1352.3448 | 329.0526 | A | 1352.3378 | 7.303 | 135065 | 100 | 5.18 |
| 1 | 1023.2922 | 329.0525 | A | 1023.2859 | 7.219 | 106700 | 100 | 6.16 |

TABLE S2
LC/MS analysis of 3′biotin-labeled RNA #1 after isolation by streptavidin beads
followed by subsequent chemical degradation (5′unlabeled mass ladder components, RNA #1).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6024.8778 | 249.0862 | A | 6024.8483 | 7.664 | 14325731 | 100 | 4.90 |
| 18 | 5775.7916 | 329.0525 | A | 5775.7522 | 7.701 | 457844 | 86.8 | 6.82 |
| 17 | 5446.7391 | 329.0525 | A | 5446.6965 | 7.411 | 417145 | 100 | 7.82 |
| 16 | 5117.6866 | 329.0525 | A | 5117.6572 | 7.105 | 490290 | 100 | 5.74 |
| 15 | 4788.6341 | 305.0413 | C | 4788.606 | 6.685 | 728135 | 100 | 5.87 |
| 14 | 4483.5928 | 305.0413 | C | 4483.5657 | 6.428 | 481770 | 100 | 6.04 |
| 13 | 4178.5515 | 329.0525 | A | 4178.5286 | 6.183 | 297514 | 100 | 5.48 |
| 12 | 3849.499 | 345.0475 | G | 3849.4787 | 5.653 | 518403 | 100 | 5.27 |
| 11 | 3504.4515 | 306.0253 | U | 3504.4331 | 5.238 | 614494 | 100 | 5.25 |
| 10 | 3198.4262 | 305.0413 | C | 3198.4106 | 4.785 | 524613 | 99.7 | 4.88 |
| 9 | 2893.3849 | 329.0525 | A | 2893.3714 | 4.341 | 373933 | 100 | 4.67 |
| 8 | 2564.3324 | 345.0474 | G | 2564.3219 | 3.458 | 509219 | 100 | 4.09 |
| 7 | 2219.285 | 306.0253 | U | 2219.2752 | 2.84 | 579139 | 100 | 4.42 |
| 6 | 1913.2597 | 305.0413 | C | 1913.2521 | 2.081 | 466058 | 100 | 3.97 |
| 5 | 1608.2184 | 306.0253 | U | 1608.2123 | 1.375 | 372038 | 80 | 3.79 |
| 4 | 1302.1931 | 329.0525 | A | 1302.1878 | 0.925 | 240613 | 100 | 4.07 |
| 3 | 973.1406 | 305.0413 | C | 973.1367 | 0.765 | 208989 | 100 | 4.01 |
| 2 | 668.0993 | 345.0474 | G | 668.0955 | 0.652 | 26061 | 100 | 5.69 |
| 1 | 323.0519 | 305.0413 | C | NA* | NA | NA | NA | NA |

*NA: Not Analyzed. The 350 Da threshold was set to minimize background ions from the elution buffers. Otherwise, we would predominantly detect HFIP and DPA ions. Thus, the masses which are smaller than 350 Da were not detected.

TABLE S3
LC/MS analysis of 5′biotin-labeled RNA #1 (5′labeled mass ladder components, RNA#1).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6600.0415 | 249.0862 | A | 6600.0153 | 10.113 | 1468018 | 100 | 3.97 |
| 18 | 6350.9553 | 329.0525 | A | 6350.9006 | 10.094 | 139388 | 80 | 8.61 |
| 17 | 6021.9028 | 329.0525 | A | 6021.8665 | 9.957 | 152155 | 80 | 6.03 |
| 16 | 5692.8503 | 329.0525 | A | 5692.8225 | 9.806 | 122377 | 83.6 | 4.88 |
| 15 | 5363.7978 | 305.0413 | C | 5363.7567 | 9.594 | 255396 | 100 | 7.66 |
| 14 | 5058.7565 | 305.0413 | C | 5058.732 | 9.508 | 169499 | 80 | 4.84 |
| 13 | 4753.7152 | 329.0525 | A | 4753.6944 | 9.449 | 121869 | 95.8 | 4.38 |
| 12 | 4424.6627 | 345.0475 | G | 4424.6389 | 9.204 | 222046 | 100 | 5.38 |
| 11 | 4079.6152 | 306.0253 | U | 4079.5902 | 9.067 | 296271 | 100 | 6.13 |
| 10 | 3773.5899 | 305.0413 | C | 3773.5679 | 8.937 | 249085 | 100 | 5.83 |
| 9 | 3468.5486 | 329.0525 | A | 3468.5308 | 8.838 | 185624 | 100 | 5.13 |
| 8 | 3139.4961 | 345.0474 | G | 3139.4834 | 8.507 | 319911 | 100 | 4.05 |
| 7 | 2794.4487 | 306.0253 | U | 2794.436 | 8.288 | 380189 | 100 | 4.54 |
| 6 | 2488.4234 | 305.0413 | C | 2488.4134 | 8.073 | 317954 | 100 | 4.02 |
| 5 | 2183.3821 | 306.0253 | U | 2183.3725 | 7.863 | 305479 | 100 | 4.40 |
| 4 | 1877.3568 | 329.0525 | A | 1877.3489 | 7.642 | 222446 | 100 | 4.21 |
| 3 | 1548.3043 | 305.0413 | C | 1548.2982 | 7.088 | 361254 | 100 | 3.94 |
| 2 | 1243.263 | 345.0474 | G | 1243.2575 | 6.798 | 162972 | 100 | 4.42 |
| 1 | 898.2156 | 305.0413 | C | 898.2105 | 6.880 | 88421 | 100 | 5.68 |

TABLE S4
LC/MS analysis of 5 biotin-labeled RNA #2 (5′labeled mass ladder components, RNA #2).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6898.0505 | 225.075 | C | 6898.0210 | 10.014 | 3995416 | 100 | 4.28 |
| 19 | 6672.9755 | 345.0474 | G | 6673.4755 | 10.115 | 92706 | 80 | 74.93 |
| 18 | 6327.9281 | 305.0413 | C | 6327.8894 | 10.117 | 108088 | 80 | 6.12 |
| 17 | 6022.8868 | 329.0525 | A | 6022.8313 | 10.104 | 133027 | 100 | 9.21 |
| 16 | 5693.8343 | 306.0253 | U | 5693.7870 | 9.920 | 68281 | 80 | 8.31 |
| 15 | 5387.809 | 305.0413 | C | 5387.7785 | 9.850 | 167081 | 80 | 5.66 |
| 14 | 5082.7677 | 306.0253 | U | 5082.7314 | 9.784 | 170198 | 100 | 7.14 |
| 13 | 4776.7424 | 345.0474 | G | 4776.7210 | 9.695 | 114657 | 98.8 | 4.48 |
| 12 | 4431.695 | 329.0526 | A | 4431.6685 | 9.629 | 143358 | 91.5 | 5.98 |
| 11 | 4102.6424 | 305.0412 | C | 4102.6199 | 9.367 | 245033 | 100 | 5.48 |
| 10 | 3797.6012 | 306.0253 | U | 3797.5819 | 9.264 | 184127 | 100 | 5.08 |
| 9 | 3491.5759 | 345.0475 | G | 3491.5567 | 9.131 | 91691 | 100 | 5.50 |
| 8 | 3146.5284 | 329.0525 | A | 3146.5054 | 9.028 | 187937 | 100 | 7.31 |
| 7 | 2817.4759 | 305.0413 | C | 2817.4633 | 8.675 | 288050 | 100 | 4.47 |
| 6 | 2512.4346 | 305.0413 | C | 2512.4233 | 8.509 | 138698 | 100 | 4.50 |
| 5 | 2207.3933 | 305.0413 | C | 2207.3835 | 8.335 | 192998 | 100 | 4.44 |
| 4 | 1902.352 | 345.0474 | G | 1902.3433 | 8.161 | 149466 | 100 | 4.57 |
| 3 | 1557.3046 | 329.0525 | A | 1557.2976 | 8.042 | 133349 | 100 | 4.49 |
| 2 | 1228.2521 | 306.0253 | U | 1228.2455 | 7.618 | 188828 | 100 | 5.37 |
| 1 | 922.2268 | 329.0525 | A | 922.2213 | 7.434 | 86674 | 100 | 5.96 |

TABLE S5
LC/MS analysis of 3′biotin-labeled RNA#1 (3′labeled mass ladder components, RNA #1).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6781.0733 | 305.0413 | C | 6781.0476 | 9.552 | 1439108 | 100 | 3.79 |
| 18 | 6476.0320 | 345.0474 | G | 6475.9807 | 9.525 | 256582 | 90.2 | 7.92 |
| 17 | 6130.9846 | 305.0413 | C | 6130.9052 | 9.466 | 208256 | 80 | 12.95 |
| 16 | 5825.9433 | 329.0525 | A | 5825.8968 | 9.593 | 309638 | 98.8 | 7.98 |
| 15 | 5496.8908 | 306.0253 | U | 5496.8429 | 9.198 | 241141 | 95.4 | 8.71 |
| 14 | 5190.8655 | 305.0413 | C | 5190.8331 | 9.058 | 407162 | 100 | 6.24 |
| 13 | 4885.8242 | 306.0253 | U | 4885.7984 | 8.959 | 408024 | 100 | 5.28 |
| 12 | 4579.7989 | 345.0475 | G | 4579.7712 | 8.937 | 431600 | 100 | 6.05 |
| 11 | 4234.7514 | 329.0525 | A | 4234.7262 | 8.976 | 490860 | 100 | 5.95 |
| 10 | 3905.6989 | 305.0413 | C | 3905.6751 | 8.419 | 257315 | 100 | 6.09 |
| 9 | 3600.6576 | 306.0253 | U | 3600.638 | 8.271 | 336323 | 100 | 5.44 |
| 8 | 3294.6323 | 345.0474 | G | 3294.6175 | 8.228 | 433533 | 100 | 4.49 |
| 7 | 2949.5849 | 329.0525 | A | 2949.5701 | 8.205 | 431168 | 100 | 5.02 |
| 6 | 2620.5324 | 305.0413 | C | 2620.5193 | 7.374 | 163100 | 100 | 5.00 |
| 5 | 2315.4911 | 305.0413 | C | 2315.4814 | 7.192 | 366354 | 100 | 4.19 |
| 4 | 2010.4498 | 329.0525 | A | 2010.4386 | 7.528 | 703696 | 100 | 5.57 |
| 3 | 1681.3973 | 329.0525 | A | 1681.3894 | 7.274 | 439312 | 100 | 4.70 |
| 2 | 1352.3448 | 329.0526 | A | 1352.3375 | 7.236 | 326818 | 100 | 5.40 |
| 1 | 1023.2922 | 1023.2922 | A | 1023.2871 | 7.156 | 229472 | 100 | 4.98 |

TABLE S6
LC/MS analysis of 5′Cy3-labeled RNA#1 (5′labeled mass ladder components, RNA #1).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6699.1470 | 249.0862 | A | 6699.1256 | 18.524 | 5427844 | 100 | 3.19 |
| 18 | 6450.0608 | 329.0525 | A | 6449.9835 | 18.332 | 53422 | 62.7 | 11.98 |
| 17 | 6121.0083 | 329.0525 | A | 6120.8891 | 18.514 | 169274 | 65.2 | 19.47 |
| 16 | 5791.9558 | 329.0525 | A | 5791.9216 | 18.714 | 144098 | 80 | 5.90 |
| 15 | 5462.9033 | 305.0413 | C | 5462.8752 | 18.912 | 209335 | 80 | 5.14 |
| 14 | 5157.8620 | 305.0413 | C | 5157.8321 | 19.171 | 126348 | 88 | 5.80 |
| 13 | 4852.8207 | 329.0525 | A | 4852.7935 | 19.463 | 73470 | 77.2 | 5.60 |
| 12 | 4523.7682 | 345.0475 | G | 4523.7443 | 19.727 | 116108 | 80 | 5.28 |
| 11 | 4178.7207 | 306.0253 | U | 4178.7014 | 20.053 | 150111 | 79.4 | 4.62 |
| 10 | 3872.6954 | 305.0413 | C | 3872.6719 | 20.452 | 67114 | 60 | 6.07 |
| 9 | 3567.6541 | 329.0525 | A | 3567.6422 | 20.91 | 36809 | 55.9 | 3.34 |
| 8 | 3238.6016 | 345.0474 | G | 3238.5865 | 21.394 | 96534 | 92.7 | 4.66 |
| 7 | 2893.5542 | 306.0253 | U | 2893.5415 | 22.048 | 102530 | 80 | 4.39 |
| 6 | 2587.5289 | 305.0413 | C | 2587.5194 | 22.816 | 35118 | 60.7 | 3.67 |
| 5 | 2282.4876 | 306.0253 | U | 2282.4795 | 23.767 | 35793 | 86.2 | 3.55 |
| 4 | 1976.4623 | 329.0525 | A | 1976.4542 | 24.828 | 202040 | 100 | 4.10 |
| 3 | 1647.4098 | 305.0413 | C | 1647.4021 | 26.428 | 220072 | 100 | 4.67 |
| 2 | 1342.3685 | 345.0474 | G | 1342.3610 | 28.326 | 110504 | 100 | 5.59 |
| 1 | 997.3210 | 305.0413 | C | NA* | NA | NA | NA | NA |

TABLE S7
LC/MS analysis of a 1 ψ-containing RNA #12 (ψ unconverted mass
ladder components from 5′ to 3′, RNA #12).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6345.9028 | 265.0811 | G | 6345.9217 | 11.736 | 41088112 | 100 | −2.98 |
| 19 | 6080.8217 | 329.0525 | A | 6080.8255 | 11.769 | 2582596 | 100 | −0.62 |
| 18 | 5751.7692 | 345.0474 | G | 5751.7749 | 11.496 | 2169051 | 100 | −0.99 |
| 17 | 5406.7218 | 306.0253 | U | 5406.7209 | 11.315 | 2126771 | 100 | 0.17 |
| 16 | 5100.6965 | 319.057 | m⁵C | 5100.6941 | 11.167 | 1149416 | 100 | 0.47 |
| 15 | 4781.6395 | 329.0525 | A | 4781.6402 | 10.970 | 2692877 | 100 | −0.15 |
| 14 | 4452.5870 | 306.0253 | U | 4452.5866 | 10.566 | 5448251 | 100 | 0.09 |
| 13 | 4146.5617 | 306.0253 | U | 4146.5603 | 10.343 | 4115258 | 100 | 0.34 |
| 12 | 3840.5364 | 329.0526 | A | 3840.5352 | 10.141 | 2038738 | 100 | 0.31 |
| 11 | 3511.4838 | 305.0413 | C | 3511.4836 | 9.610 | 1167942 | 100 | 0.06 |
| 10 | 3206.4425 | 305.0412 | C | 3206.4401 | 9.331 | 3422282 | 100 | 0.75 |
| 9 | 2901.4013 | 329.0526 | A | 2901.3988 | 9.067 | 2391922 | 100 | 0.86 |
| 8 | 2572.3487 | 306.0253 | Unconverted ψ | 2572.3468 | 8.328 | 4952174 | 100 | 0.74 |
| 7 | 2266.3234 | 306.0253 | U | 2266.3215 | 7.944 | 4534905 | 100 | 0.84 |
| 6 | 1960.2981 | 345.0474 | G | 1960.2956 | 7.360 | 3437270 | 100 | 1.28 |
| 5 | 1615.2507 | 305.0413 | C | 1615.2481 | 6.693 | 4151449 | 100 | 1.61 |
| 4 | 1310.2094 | 305.0413 | C | 1310.2062 | 5.915 | 1289241 | 87 | 2.44 |
| 3 | 1005.1681 | 329.0525 | A | 1005.1655 | 4.416 | 913589 | 100 | 2.59 |
| 2 | 676.1156 | 329.0525 | A | 676.1140 | 3.321 | 748977 | 100 | 2.37 |
| 1 | 347.0631 | 329.0525 | A | NA* | NA | NA | NA | NA |

TABLE S8
LC/MS analysis of a 1 ψ-containing RNA #12 (ψ unconverted mass
ladder components from 3′ to 5′, RNA #12).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6345.9028 | 329.0525 | A | 6345.9069 | 11.361 | 91693 | 61.1 | −0.65 |
| 19 | 6016.8503 | 329.0525 | A | 6016.856 | 11.603 | 2102227 | 96 | −0.95 |
| 18 | 5687.7978 | 329.0525 | A | 5687.8032 | 11.149 | 1349414 | 100 | −0.95 |
| 17 | 5358.7453 | 305.0413 | C | 5358.7538 | 10.493 | 1095672 | 100 | −1.59 |
| 16 | 5053.7040 | 305.0413 | C | 5053.7053 | 10.247 | 1906586 | 100 | −0.26 |
| 15 | 4748.6627 | 345.0475 | G | 4748.6638 | 10.082 | 2832083 | 100 | −0.23 |
| 14 | 4403.6152 | 306.0253 | U | 4403.6162 | 9.655 | 1017645 | 100 | −0.23 |
| 13 | 4097.5899 | 306.0253 | Unconverted ψ | 4097.5897 | 9.281 | 2438044 | 100 | 0.05 |
| 12 | 3791.5646 | 329.0525 | A | 3791.5638 | 9.613 | 6450776 | 100 | 0.21 |
| 11 | 3462.5121 | 305.0413 | C | 3462.511 | 8.533 | 2959433 | 100 | 0.32 |
| 10 | 3157.4708 | 305.0413 | C | 3157.4687 | 8.247 | 4281684 | 100 | 0.67 |
| 9 | 2852.4295 | 329.0525 | A | 2852.4279 | 8.384 | 6732016 | 100 | 0.56 |
| 8 | 2523.3770 | 306.0253 | U | 2523.3752 | 7.06 | 3639095 | 100 | 0.71 |
| 7 | 2217.3517 | 306.0253 | U | 2217.3496 | 6.547 | 5142524 | 100 | 0.95 |
| 6 | 1911.3264 | 329.0525 | A | 1911.3234 | 5.628 | 148978 | 100 | 1.57 |
| 5 | 1582.2739 | 319.057 | m⁵C | 1582.271 | 4.694 | 2365111 | 100 | 1.83 |
| 4 | 1263.2169 | 306.0253 | U | 1263.216 | 1.392 | 1025750 | 100 | 0.71 |
| 3 | 957.1916 | 345.0474 | G | 957.1909 | 1.354 | 1030368 | 100 | 0.73 |
| 2 | 612.1442 | 329.0525 | A | 612.1432 | 1.334 | 609338 | 100 | 1.63 |
| 1 | 283.0917 | 345.0475 | G | NA* | NA | NA | NA | NA |

TABLE S9
LC/MS analysis of a 1 ψ-containing RNA #12 (mass ladder components with
CMC-converted ψ from 5′ to 3′, 20 nt RNA)

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6597.1025 | 265.0811 | G | 6597.1125 | 13.985 | 60627484 | 100 | −1.52 |
| 19 | 6332.0214 | 329.0525 | A | 6332.0201 | 13.979 | 1541470 | 100 | 0.21 |
| 18 | 6002.9689 | 345.0474 | G | 6002.9756 | 13.816 | 2147847 | 88.6 | −1.12 |
| 17 | 5657.9215 | 306.0253 | U | 5657.9243 | 13.742 | 2608610 | 100 | −0.49 |
| 16 | 5351.8962 | 319.057 | m⁵C | 5351.8960 | 13.695 | 2110248 | 100 | 0.04 |
| 15 | 5032.8392 | 329.0525 | A | 5032.8400 | 13.633 | 1907945 | 100 | −0.16 |
| 14 | 4703.7867 | 306.0253 | U | 4703.7861 | 13.394 | 4110706 | 88.3 | 0.13 |
| 13 | 4397.7614 | 306.0253 | U | 4397.7599 | 13.320 | 2867370 | 100 | 0.34 |
| 12 | 4091.7361 | 329.0526 | A | 4091.7361 | 13.283 | 1855682 | 100 | 0.00 |
| 11 | 3762.6835 | 305.0413 | C | 3762.6830 | 12.962 | 2817838 | 100 | 0.13 |
| 10 | 3457.6422 | 305.0412 | C | 3457.6396 | 12.878 | 1149319 | 100 | 0.75 |
| 9 | 3152.6010 | 329.0526 | A | 3152.5974 | 12.934 | 746862 | 100 | 1.14 |
| 8 | 2823.5485 | 557.2251 | Converted ψ | 2823.5455 | 12.380 | 2149383 | 100 | 1.06 |
| 7 | 2266.3234 | 306.0253 | U | 2266.3213 | 7.944 | 4767282 | 100 | 0.93 |
| 6 | 1960.2981 | 345.0474 | G | 1960.2956 | 7.360 | 3433416 | 100 | 1.28 |
| 5 | 1615.2507 | 305.0413 | C | 1615.2481 | 6.694 | 4174772 | 100 | 1.61 |
| 4 | 1310.2094 | 305.0413 | C | 1310.2071 | 5.917 | 806139 | 87 | 1.76 |
| 3 | 1005.1681 | 329.0525 | A | 1005.1655 | 4.416 | 913589 | 100 | 2.59 |
| 2 | 676.1156 | 329.0525 | A | 676.1140 | 3.321 | 743305 | 100 | 2.37 |
| 1 | 347.0631 | 329.0525 | A | NA* | NA | NA | NA | NA |

TABLE S10
LC/MS analysis of a 1 ψ-containing RNA #12 (mass ladder components with
CMC-converted ψ from 3′ to 5′, RNA #12)

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6597.1025 | 329.0525 | A | 6597.1125 | 13.985 | 60627484 | 100 | −1.52 |
| 19 | 6268.0500 | 329.0525 | A | 6268.0571 | 13.936 | 2514888 | 95.7 | −1.13 |
| 18 | 5938.9975 | 329.0525 | A | 5939.0021 | 13.618 | 919334 | 80 | −0.77 |
| 17 | 5609.9450 | 305.0413 | C | 5609.9509 | 13.027 | 550752 | 100 | −1.05 |
| 16 | 5304.9037 | 305.0413 | C | 5304.9018 | 12.95 | 1145236 | 100 | 0.36 |
| 15 | 4999.8624 | 345.0475 | G | 4999.8628 | 13.09 | 1603456 | 100 | −0.08 |
| 14 | 4654.8150 | 306.0253 | U | 4654.8165 | 12.976 | 1028627 | 100 | −0.32 |
| 13 | 4348.7897 | 557.2251 | Converted ψ | 4348.7878 | 12.747 | 1061149 | 100 | 0.44 |
| 12 | 3791.5646 | 329.0525 | A | 3791.5638 | 9.613 | 6450776 | 100 | 0.21 |
| 11 | 3462.5121 | 305.0413 | C | 3462.511 | 8.533 | 2959433 | 100 | 0.32 |
| 10 | 3157.4708 | 305.0413 | C | 3157.4687 | 8.247 | 4281684 | 100 | 0.67 |
| 9 | 2852.4295 | 329.0525 | A | 2852.4279 | 8.384 | 6732016 | 100 | 0.56 |
| 8 | 2523.3770 | 306.0253 | U | 2523.3752 | 7.06 | 3639095 | 100 | 0.71 |
| 7 | 2217.3517 | 306.0253 | U | 2217.3496 | 6.547 | 5142524 | 100 | 0.95 |
| 6 | 1911.3264 | 329.0525 | A | 1911.3234 | 5.628 | 148978 | 100 | 1.57 |
| 5 | 1582.2739 | 319.057 | m⁵C | 1582.271 | 4.694 | 2365111 | 100 | 1.83 |
| 4 | 1263.2169 | 306.0253 | U | 1263.216 | 1.392 | 1025750 | 100 | 0.71 |
| 3 | 957.1916 | 345.0474 | G | 957.1909 | 1.355 | 1052036 | 100 | 0.73 |
| 2 | 612.1442 | 329.0525 | A | 612.1432 | 1.334 | 609338 | 100 | 1.63 |
| 1 | 283.0917 | 345.0475 | G | NA* | NA | NA | NA | NA |

TABLE S11
LC/MS analysis of a 2 ψ-containing RNA #13 (ψ unconverted mass
ladder components from 5′ to 3′, RNA #13).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6331.8871 | 265.0811 | G | 6331.9010 | 11.627 | 20815662 | 100 | −2.20 |
| 19 | 6066.8060 | 329.0525 | A | 6066.8121 | 11.661 | 1640168 | 99.3 | −1.01 |
| 18 | 5737.7535 | 345.0474 | G | 5737.7570 | 11.382 | 885613 | 80 | −0.61 |
| 17 | 5392.7061 | 306.0253 | Unconverted ψ | 5392.7060 | 11.212 | 617277 | 100 | 0.02 |
| 16 | 5086.6808 | 305.0413 | C | 5086.6829 | 11.082 | 2141353 | 100 | −0.41 |
| 15 | 4781.6395 | 329.0525 | A | 4781.6267 | 10.759 | 26031 | 75.4 | 2.68 |
| 14 | 4452.5870 | 306.0253 | U | 4452.5872 | 10.522 | 3256295 | 100 | −0.04 |
| 13 | 4146.5617 | 306.0253 | U | 4146.5608 | 10.294 | 2867802 | 100 | 0.22 |
| 12 | 3840.5364 | 329.0526 | A | 3840.5345 | 10.089 | 1804456 | 100 | 0.49 |
| 11 | 3511.4838 | 305.0413 | C | 3511.4825 | 9.545 | 3618243 | 100 | 0.37 |
| 10 | 3206.4425 | 305.0412 | C | 3206.4408 | 9.254 | 2325449 | 100 | 0.53 |
| 9 | 2901.4013 | 329.0526 | A | 2901.3978 | 8.965 | 1647914 | 100 | 1.21 |
| 8 | 2572.3487 | 306.0253 | Unconverted ψ | 2572.3461 | 8.205 | 3697493 | 100 | 1.01 |
| 7 | 2266.3234 | 306.0253 | U | 2266.3205 | 7.822 | 3317588 | 100 | 1.28 |
| 6 | 1960.2981 | 345.0474 | G | 1960.2952 | 7.245 | 2415197 | 100 | 1.48 |
| 5 | 1615.2507 | 305.0413 | C | 1615.2480 | 6.605 | 2827204 | 100 | 1.67 |
| 4 | 1310.2094 | 305.0413 | C | 1310.2060 | 5.804 | 1306273 | 80 | 2.60 |
| 3 | 1005.1681 | 329.0525 | A | 1005.1658 | 4.496 | 867786 | 100 | 2.29 |
| 2 | 676.1156 | 329.0525 | A | 676.1140 | 3.231 | 662092 | 100 | 2.37 |
| 1 | 347.0630 | 329.0525 | A | NA* | NA | NA | NA | NA |

TABLE S12
LC/MS analysis of a 2 ψ-containing RNA #13 (mass ladder components with 1
CMC-converted ψ from 5′ to 3′, 20 nt RNA #13).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6583.0869 | 265.0811 | G | 6583.0981 | 13.829 | 35962424 | 100 | −1.70 |
| 19 | 6318.0058 | 329.0525 | A | 6318.0114 | 13.829 | 938044 | 100 | −0.89 |
| 18 | 5988.9533 | 345.0474 | G | 5988.9552 | 13.654 | 602824 | 96.1 | −0.32 |
| 17 | 5643.9059 | 306.0253 | Unconverted ψ | 5643.9107 | 13.573 | 1578612 | 80 | −0.85 |
| 16 | 5337.8806 | 305.0413 | C | 5337.8852 | 13.573 | 1563724 | 100 | −0.86 |
| 15 | 5032.8393 | 329.0525 | A | 5032.8468 | 13.541 | 991863 | 100 | −1.49 |
| 14 | 4703.7868 | 306.0253 | U | 4703.7876 | 13.308 | 1970261 | 100 | −0.17 |
| 13 | 4397.7615 | 306.0253 | U | 4397.7601 | 13.230 | 817755 | 100 | 0.32 |
| 12 | 4091.7362 | 329.0526 | A | 4091.7338 | 13.190 | 330683 | 98.1 | 0.59 |
| 11 | 3762.6836 | 305.0413 | C | 3762.6827 | 12.884 | 1591068 | 100 | 0.24 |
| 10 | 3457.6423 | 305.0412 | C | 3457.6403 | 12.806 | 1110204 | 99.5 | 0.58 |
| 9 | 3152.6011 | 329.0526 | A | 3152.5988 | 12.857 | 512332 | 100 | 0.73 |
| 8 | 2823.5485 | 557.2251 | Converted ψ | 2823.5457 | 12.325 | 1193480 | 100 | 0.99 |
| 7 | 2266.3234 | 306.0253 | U | 2266.3205 | 7.822 | 3317588 | 100 | 1.28 |
| 6 | 1960.2981 | 345.0474 | G | 1960.2952 | 7.245 | 2415197 | 100 | 1.48 |
| 5 | 1615.2507 | 305.0413 | C | 1615.2480 | 6.605 | 2827204 | 100 | 1.67 |
| 4 | 1310.2094 | 305.0413 | C | 1310.206 | 5.804 | 1306273 | 80 | 2.60 |
| 3 | 1005.1681 | 329.0525 | A | 1005.1658 | 4.496 | 867786 | 100 | 2.29 |
| 2 | 676.1156 | 329.0525 | A | 676.1140 | 3.231 | 662092 | 100 | 2.37 |
| 1 | 347.0630 | 329.0525 | A | NA* | NA | NA | NA | NA |

TABLE S13
LC/MS analysis of a 2 ψ-containing RNA #13 (mass ladder components with
1 CMC-converted ψ from 5′ to 3′, RNA #13).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6583.0869 | 265.0811 | G | 6583.0981 | 13.829 | 35962424 | 100 | −1.70 |
| 19 | 6318.0058 | 329.0525 | A | 6318.0114 | 13.829 | 938044 | 100 | −0.89 |
| 18 | 5988.9533 | 345.0474 | G | 5988.9552 | 13.654 | 602824 | 96.1 | −0.32 |
| 17 | 5643.9059 | 557.2251 | Converted ψ | 5643.9107 | 13.573 | 1578612 | 80 | −0.85 |
| 16 | 5086.6808 | 305.0413 | C | 5086.6827 | 11.08 | 1427810 | 100 | −0.37 |
| 15 | 4781.6395 | 329.0525 | A | 4781.6412 | 10.926 | 1523517 | 100 | −0.36 |
| 14 | 4452.587 | 306.0253 | U | 4452.588 | 10.522 | 2085205 | 100 | −0.22 |
| 13 | 4146.5617 | 306.0253 | U | 4146.5609 | 10.294 | 2788426 | 100 | 0.19 |
| 12 | 3840.5364 | 329.0526 | A | 3840.5345 | 10.084 | 1938977 | 100 | 0.49 |
| 11 | 3511.4838 | 305.0413 | C | 3511.4816 | 9.546 | 3088818 | 100 | 0.63 |
| 10 | 3206.4425 | 305.0412 | C | 3206.4409 | 9.253 | 2028277 | 100 | 0.50 |
| 9 | 2901.4013 | 329.0526 | A | 2901.3977 | 8.965 | 1489932 | 100 | 1.24 |
| 8 | 2572.3487 | 306.0253 | Unconverted ψ | 2572.3461 | 8.205 | 3716588 | 100 | 1.01 |
| 7 | 2266.3234 | 306.0253 | U | 2266.3205 | 7.822 | 3317588 | 100 | 1.28 |
| 6 | 1960.2981 | 345.0474 | G | 1960.2952 | 7.245 | 2415197 | 100 | 1.48 |
| 5 | 1615.2507 | 305.0413 | C | 1615.248 | 6.605 | 2827204 | 100 | 1.67 |
| 4 | 1310.2094 | 305.0413 | C | 1310.206 | 5.804 | 1306273 | 80 | 2.60 |
| 3 | 1005.1681 | 329.0525 | A | 1005.1658 | 4.496 | 867786 | 100 | 2.29 |
| 2 | 676.1156 | 329.0525 | A | 676.114 | 3.231 | 662092 | 100 | 2.37 |
| 1 | 347.0631 | 329.0525 | A | NA* | NA | NA | NA | NA |

TABLE S14
LC/MS analysis of a 2 ψ-containing RNA #13 (mass ladder components
with 2 CMC-converted ψ from 5′ to RNA #13).

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6834.2866 | 265.0811 | G | 6834.2945 | 15.887 | 10647840 | 100 | −1.16 |
| 19 | 6569.2055 | 329.0525 | A | 6569.2283 | 15.694 | 22547 | 72.5 | −3.47 |
| 18 | 6240.1530 | 345.0474 | G | 6241.1635 | 15.787 | 151235 | 79.1 | −161.94 |
| 17 | 5895.1056 | 557.2251 | Converted ψ | 5895.0646 | 15.870 | 3373 | 53.3 | 6.95 |
| 16 | 5337.8805 | 305.0413 | C | 5337.8852 | 13.573 | 1563724 | 100 | −0.88 |
| 15 | 5032.8392 | 329.0525 | A | 5032.8468 | 13.541 | 991863 | 100 | −1.51 |
| 14 | 4703.7867 | 306.0253 | U | 4703.7876 | 13.308 | 1970261 | 100 | −0.19 |
| 13 | 4397.7614 | 306.0253 | U | 4397.7601 | 13.230 | 817755 | 100 | 0.30 |
| 12 | 4091.7361 | 329.0526 | A | 4091.7338 | 13.190 | 330683 | 98.1 | 0.56 |
| 11 | 3762.6835 | 305.0413 | C | 3762.6827 | 12.884 | 1591068 | 100 | 0.21 |
| 10 | 3457.6422 | 305.0412 | C | 3457.6403 | 12.806 | 1110204 | 99.5 | 0.55 |
| 9 | 3152.6010 | 329.0526 | A | 3152.5988 | 12.857 | 512332 | 100 | 0.70 |
| 8 | 2823.5484 | 557.2251 | Converted ψ | 2823.5457 | 12.325 | 1193480 | 100 | 0.96 |
| 7 | 2266.3233 | 306.0253 | U | 2266.3205 | 7.822 | 3317588 | 100 | 1.24 |
| 6 | 1960.2980 | 345.0474 | G | 1960.2952 | 7.245 | 2415197 | 100 | 1.43 |
| 5 | 1615.2506 | 305.0413 | C | 1615.248 | 6.605 | 2827204 | 100 | 1.61 |
| 4 | 1310.2093 | 305.0413 | C | 1310.206 | 5.804 | 1306273 | 80 | 2.52 |
| 3 | 1005.1680 | 329.0525 | A | 1005.1658 | 4.496 | 867786 | 100 | 2.19 |
| 2 | 676.1155 | 329.0525 | A | 676.1140 | 3.231 | 662092 | 100 | 2.22 |
| 1 | 347.0630 | 329.0525 | A | NA* | NA | NA | NA | NA |

TABLE S15
LC/MS analysis of 3′biotin-labeled RNA #1, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6781.0733 | 305.0413 | C | 6781.0413 | 9.752 | 16819442 | 100 | 4.72 |
| 18 | 6476.0320 | 345.0474 | G | 6475.9924 | 9.717 | 247965 | 84 | 6.11 |
| 17 | 6130.9846 | 305.0413 | C | 6130.9398 | 9.662 | 178841 | 80 | 7.31 |
| 16 | 5825.9433 | 329.0525 | A | 5825.9037 | 9.782 | 510096 | 80 | 6.80 |
| 15 | 5496.8908 | 306.0253 | U | 5496.8566 | 9.383 | 262486 | 99 | 6.22 |
| 14 | 5190.8655 | 305.0413 | C | 5190.8364 | 9.241 | 349988 | 100 | 5.61 |
| 13 | 4885.8242 | 306.0253 | U | 4885.7908 | 9.135 | 356118 | 100 | 6.84 |
| 12 | 4579.7989 | 345.0475 | G | 4579.7738 | 9.109 | 386687 | 100 | 5.48 |
| 11 | 4234.7514 | 329.0525 | A | 4234.7271 | 9.145 | 305380 | 100 | 5.74 |
| 10 | 3905.6989 | 305.0413 | C | 3905.6749 | 8.575 | 145505 | 96 | 6.14 |
| 9 | 3600.6576 | 306.0253 | U | 3600.6373 | 8.420 | 195308 | 100 | 5.64 |
| 8 | 3294.6323 | 345.0474 | G | 3294.6165 | 8.370 | 125991 | 100 | 4.80 |
| 7 | 2949.5849 | 329.0525 | A | 2949.5716 | 8.339 | 106993 | 100 | 4.51 |
| 6 | 2620.5324 | 305.0413 | C | 2620.5193 | 7.492 | 90629 | 100 | 5.00 |
| 5 | 2315.4911 | 305.0413 | C | 2315.4814 | 7.299 | 163692 | 100 | 4.19 |
| 4 | 2010.4498 | 329.0525 | A | 2010.4388 | 7.625 | 279963 | 100 | 5.47 |
| 3 | 1681.3973 | 329.0525 | A | 1681.3891 | 7.354 | 183827 | 100 | 4.88 |
| 2 | 1352.3448 | 329.0526 | A | 1352.3378 | 7.303 | 135065 | 100 | 5.18 |
| 1 | 1023.2922 | 329.0525 | A | 1023.2859 | 7.219 | 106700 | 100 | 6.16 |

TABLE S16
LC/MS analysis of 3′biotin-labeled RNA #2, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7079.0823 | 329.2088 | A | 7079.0519 | 9.695 | 15887400 | 100 | 4.29 |
| 19 | 6750.0298 | 306.1667 | U | 6749.9576 | 9.422 | 103400 | 80 | 10.70 |
| 18 | 6444.0045 | 329.2088 | A | 6443.9541 | 9.504 | 292394 | 91.2 | 7.82 |
| 17 | 6114.9519 | 345.2077 | G | 6114.9026 | 9.156 | 99684 | 87 | 8.06 |
| 16 | 5769.9045 | 305.1828 | C | 5769.8585 | 9.020 | 146499 | 80 | 7.97 |
| 15 | 5464.8632 | 305.1828 | C | 5464.8200 | 8.887 | 63438 | 80 | 7.91 |
| 14 | 5159.8219 | 305.1827 | C | 5159.8026 | 8.769 | 284881 | 100 | 3.74 |
| 13 | 4854.7806 | 329.2088 | A | 4854.7562 | 8.879 | 336079 | 100 | 5.03 |
| 12 | 4525.7281 | 345.2078 | G | 4525.7034 | 8.413 | 242815 | 100 | 5.46 |
| 11 | 4180.6807 | 306.1667 | U | 4180.6582 | 8.181 | 208097 | 100 | 5.38 |
| 10 | 3874.6554 | 305.1828 | C | 3874.6356 | 7.962 | 274449 | 100 | 5.11 |
| 9 | 3569.6141 | 329.2087 | A | 3569.5960 | 8.083 | 385282 | 100 | 5.07 |
| 8 | 3240.5616 | 345.2078 | G | 3240.5467 | 7.440 | 238714 | 100 | 4.60 |
| 7 | 2895.5141 | 306.1668 | U | 2895.4995 | 7.096 | 215938 | 100 | 5.04 |
| 6 | 2589.4888 | 305.1827 | C | 2589.4766 | 6.736 | 291557 | 100 | 4.71 |
| 5 | 2284.4476 | 306.1668 | U | 2284.4371 | 6.523 | 322833 | 100 | 4.60 |
| 4 | 1978.4223 | 329.2088 | A | 1978.4135 | 6.382 | 360972 | 100 | 4.45 |
| 3 | 1649.3697 | 305.1827 | C | 1649.3626 | 5.238 | 129210 | 100 | 4.30 |
| 2 | 1344.3284 | 345.2078 | G | 1344.3224 | 5.191 | 163260 | 100 | 4.46 |
| 1 | 999.2810 | 305.1827 | C | 999.2753 | 5.323 | 81388 | 100 | 5.70 |

TABLE S17
LC/MS analysis of 3′biotin-labeled RNA #3, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7088.0826 | 329.0525 | A | 7088.0481 | 9.912 | 20041130 | 100 | 4.87 |
| 19 | 6759.0301 | 329.0525 | A | 6758.9787 | 9.827 | 312216 | 85.4 | 7.60 |
| 18 | 6429.9776 | 329.0525 | A | 6429.9267 | 9.575 | 270720 | 80 | 7.92 |
| 17 | 6100.9251 | 305.0413 | C | 6100.8781 | 9.174 | 239340 | 80 | 7.70 |
| 16 | 5795.8838 | 305.0413 | C | 5795.8548 | 9.071 | 488843 | 100 | 5.00 |
| 15 | 5490.8425 | 345.0475 | G | 5490.8073 | 9.044 | 673490 | 98.9 | 6.41 |
| 14 | 5145.7950 | 306.0253 | U | 5145.7622 | 8.944 | 583546 | 100 | 6.37 |
| 13 | 4839.7697 | 306.0253 | U | 4839.7411 | 8.870 | 671098 | 100 | 5.91 |
| 12 | 4533.7444 | 329.0525 | A | 4533.7210 | 8.874 | 1044860 | 100 | 5.16 |
| 11 | 4204.6919 | 305.0413 | C | 4204.6731 | 8.297 | 513780 | 100 | 4.47 |
| 10 | 3899.6506 | 305.0413 | C | 3899.6292 | 8.185 | 650568 | 100 | 5.49 |
| 9 | 3594.6093 | 329.0525 | A | 3594.5921 | 8.321 | 1203072 | 100 | 4.78 |
| 8 | 3265.5568 | 306.0253 | U | 3265.5424 | 7.668 | 797335 | 100 | 4.41 |
| 7 | 2959.5315 | 306.0253 | U | 2959.5198 | 7.482 | 1166317 | 100 | 3.95 |
| 6 | 2653.5062 | 329.0525 | A | 2653.4961 | 7.449 | 1461689 | 100 | 3.81 |
| 5 | 2324.4537 | 305.0413 | C | 2324.4451 | 6.497 | 759285 | 100 | 3.70 |
| 4 | 2019.4124 | 306.0253 | U | 2019.4051 | 6.113 | 869967 | 100 | 3.61 |
| 3 | 1713.3871 | 345.0474 | G | 1713.3810 | 5.978 | 955386 | 100 | 3.56 |
| 2 | 1368.3397 | 329.0525 | A | 1368.3338 | 6.370 | 586922 | 100 | 4.31 |
| 1 | 1039.2872 | 1039.2872 | G | 1039.2825 | 5.660 | 342316 | 100 | 4.52 |

TABLE S18
LC/MS analysis of 3′biotin-labeled RNA #4, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6985.0431 | 306.0253 | U | 6985.0207 | 11.625 | 58498820 | 100 | 3.21 |
| 19 | 6679.0178 | 345.0474 | G | 6679.9864 | 11.557 | 78870 | 100 | −145.02 |
| 18 | 6333.9704 | 306.0253 | U | 6333.9577 | 11.514 | 1165403 | 95 | 2.01 |
| 17 | 6027.9451 | 329.0526 | A | 6027.9150 | 11.707 | 3055438 | 100 | 4.99 |
| 16 | 5698.8925 | 329.0525 | A | 5698.8699 | 11.588 | 2305641 | 84.8 | 3.97 |
| 15 | 5369.8400 | 329.0525 | A | 5369.8145 | 11.201 | 1931925 | 100 | 4.75 |
| 14 | 5040.7875 | 305.0413 | C | 5040.7605 | 10.777 | 1506142 | 100 | 5.36 |
| 13 | 4735.7462 | 329.0525 | A | 4735.7232 | 11.042 | 3132367 | 100 | 4.86 |
| 12 | 4406.6937 | 306.0253 | U | 4406.6725 | 10.372 | 1761089 | 100 | 4.81 |
| 11 | 4100.6684 | 305.0413 | C | 4100.6501 | 10.171 | 2219510 | 100 | 4.46 |
| 10 | 3795.6271 | 305.0413 | C | 3795.6100 | 10.043 | 2529132 | 100 | 4.51 |
| 9 | 3490.5858 | 306.0253 | U | 3490.5716 | 10.035 | 2441434 | 100 | 4.07 |
| 8 | 3184.5605 | 329.0525 | A | 3184.5476 | 10.052 | 3440631 | 100 | 4.05 |
| 7 | 2855.5080 | 305.0413 | C | 2855.4962 | 9.308 | 1722723 | 100 | 4.13 |
| 6 | 2550.4667 | 329.0525 | A | 2550.4587 | 9.605 | 2447222 | 97.7 | 3.14 |
| 5 | 2221.4142 | 305.0413 | C | 2221.4058 | 8.474 | 1901654 | 100 | 3.78 |
| 4 | 1916.3729 | 306.0253 | U | 1916.3661 | 8.222 | 2469329 | 100 | 3.55 |
| 3 | 1610.3476 | 305.0413 | C | 1610.3419 | 7.922 | 2259370 | 100 | 3.54 |
| 2 | 1305.3063 | 306.0253 | U | 1305.3016 | 7.899 | 1603980 | 100 | 3.60 |
| 1 | 999.2810 | 305.0413 | C | 999.2770 | 8.131 | 1272190 | 100 | 4.00 |

TABLE S19
LC/MS analysis of 3′biotin-labeled RNA #5, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7073.0717 | 306.0253 | U | 7073.0472 | 12.156 | 82887552 | 100 | 3.46 |
| 19 | 6767.0464 | 329.0525 | A | 6767.0137 | 12.296 | 4576892 | 100 | 4.83 |
| 18 | 6437.9939 | 306.0253 | U | 6437.9769 | 11.923 | 2315485 | 100 | 2.64 |
| 17 | 6131.9686 | 306.0253 | U | 6131.9364 | 11.837 | 3353132 | 100 | 5.25 |
| 16 | 5825.9433 | 305.0413 | C | 5825.9117 | 11.751 | 3225051 | 100 | 5.42 |
| 15 | 5520.9020 | 329.0525 | A | 5520.8693 | 11.976 | 4163809 | 100 | 5.92 |
| 14 | 5191.8495 | 329.0525 | A | 5191.8215 | 11.751 | 3066109 | 100 | 5.39 |
| 13 | 4862.7970 | 345.0475 | G | 4862.7739 | 11.305 | 2677901 | 100 | 4.75 |
| 12 | 4517.7495 | 306.0253 | U | 4517.7279 | 11.153 | 2051199 | 100 | 4.78 |
| 11 | 4211.7242 | 306.0253 | U | 4211.7051 | 11.108 | 3646647 | 100 | 4.53 |
| 10 | 3905.6989 | 329.0525 | A | 3905.6825 | 11.163 | 4185511 | 100 | 4.20 |
| 9 | 3576.6464 | 305.0413 | C | 3576.6311 | 10.626 | 2134080 | 100 | 4.28 |
| 8 | 3271.6051 | 329.0525 | A | 3271.5909 | 10.892 | 4157558 | 100 | 4.34 |
| 7 | 2942.5526 | 305.0413 | C | 2942.5413 | 10.114 | 2150759 | 100 | 3.84 |
| 6 | 2637.5113 | 306.0253 | U | 2637.5010 | 9.986 | 2806597 | 100 | 3.91 |
| 5 | 2331.4860 | 305.0413 | C | 2331.4714 | 10.257 | 10052 | 100 | 6.26 |
| 4 | 2026.4447 | 329.0525 | A | 2026.4377 | 10.176 | 3408728 | 100 | 3.45 |
| 3 | 1697.3922 | 329.0525 | A | 1697.3861 | 9.691 | 2143607 | 100 | 3.59 |
| 2 | 1368.3397 | 345.0475 | G | 1368.3344 | 9.292 | 1254041 | 100 | 3.87 |
| 1 | 1023.2922 | 329.0525 | A | 1023.2882 | 10.603 | 1407833 | 100 | 3.91 |

TABLE S20
LC/MS analysis of 3′biotin-labeled RNA #6, showing its mass ladder components.

| Theoretical | | | Extracted data file after LC/MS analysis | | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 6954.9836 | 345.0475 | G | 6954.9496 | 9.252 | 19518152 | 100 | 4.89 |
| 19 | 6609.9361 | 305.0412 | C | 6609.8895 | 9.140 | 186061 | 80 | 7.05 |
| 18 | 6304.8949 | 345.0475 | G | 6304.8493 | 9.116 | 570614 | 95.2 | 7.23 |
| 17 | 5959.8474 | 306.0253 | U | 5959.8016 | 9.064 | 430830 | 100 | 7.68 |
| 16 | 5653.8221 | 329.0525 | A | 5653.7951 | 9.068 | 845499 | 100 | 4.78 |
| 15 | 5324.7696 | 305.0413 | C | 5324.7375 | 8.714 | 497200 | 100 | 6.03 |
| 14 | 5019.7283 | 329.0525 | A | 5019.7206 | 8.862 | 69267 | 80 | 1.53 |
| 13 | 4690.6758 | 306.0253 | U | 4690.6508 | 8.360 | 624270 | 100 | 5.33 |
| 12 | 4384.6505 | 305.0413 | C | 4384.6287 | 8.202 | 905111 | 100 | 4.97 |
| 11 | 4079.6092 | 306.0253 | U | 4079.5872 | 8.088 | 934627 | 100 | 5.39 |
| 10 | 3773.5839 | 306.0253 | U | 3773.5610 | 7.898 | 865362 | 100 | 6.07 |
| 9 | 3467.5586 | 305.0413 | C | 3467.5420 | 7.648 | 551801 | 100 | 4.79 |
| 8 | 3162.5173 | 305.0413 | C | 3162.5033 | 7.427 | 763065 | 100 | 4.43 |
| 7 | 2857.4760 | 305.0413 | C | 2857.4632 | 7.176 | 934459 | 100 | 4.48 |
| 6 | 2552.4347 | 305.0412 | C | 2552.4237 | 6.942 | 1266516 | 100 | 4.31 |
| 5 | 2247.3935 | 306.0253 | U | 2247.3841 | 6.711 | 1457982 | 100 | 4.18 |
| 4 | 1941.3682 | 306.0253 | U | 1941.3606 | 6.369 | 1784912 | 100 | 3.91 |
| 3 | 1635.3429 | 306.0254 | U | 1635.3358 | 6.162 | 1549510 | 100 | 4.34 |
| 2 | 1329.3175 | 329.0525 | A | 1329.3122 | 6.619 | 1621370 | 100 | 3.99 |
| 1 | 1000.2650 | 306.0253 | U | 1000.2615 | 5.284 | 24083 | 100 | 3.50 |

TABLE S21
LC/MS analysis of 3′biotin-labeled RNA #7, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7110.0881 | 305.0413 | C | 7110.0622 | 11.550 | 40533036 | 100 | 3.64 |
| 19 | 6805.0468 | 345.0474 | G | 6805.0163 | 11.536 | 1377944 | 100 | 4.48 |
| 18 | 6459.9994 | 305.0413 | C | 6459.9655 | 11.383 | 515259 | 95.7 | 5.25 |
| 17 | 6154.9581 | 305.0413 | C | 6154.9267 | 11.333 | 915022 | 100 | 5.10 |
| 16 | 5849.9168 | 329.0525 | A | 5849.8891 | 11.425 | 2491248 | 99.1 | 4.74 |
| 15 | 5520.8643 | 306.0253 | U | 5520.8364 | 10.963 | 957615 | 100 | 5.05 |
| 14 | 5214.8390 | 345.0475 | G | 5214.8129 | 10.913 | 1607534 | 100 | 5.00 |
| 13 | 4869.7915 | 306.0253 | U | 4869.7663 | 10.706 | 1002213 | 100 | 5.17 |
| 12 | 4563.7662 | 345.0474 | G | 4563.7450 | 10.786 | 872578 | 100 | 4.65 |
| 11 | 4218.7188 | 329.0525 | A | 4218.6990 | 10.933 | 1284822 | 100 | 4.69 |
| 10 | 3889.6663 | 306.0253 | U | 3889.6549 | 10.212 | 786209 | 100 | 2.93 |
| 9 | 3583.6410 | 305.0413 | C | 3583.6265 | 9.978 | 940944 | 100 | 4.05 |
| 8 | 3278.5997 | 305.0413 | C | 3278.5866 | 9.685 | 809912 | 100 | 4.00 |
| 7 | 2973.5584 | 305.0413 | C | 2973.5474 | 9.381 | 679854 | 100 | 3.70 |
| 6 | 2668.5171 | 345.0474 | G | 2668.5070 | 9.315 | 819030 | 100 | 3.78 |
| 5 | 2323.4697 | 345.0474 | G | 2323.4614 | 9.329 | 646645 | 100 | 3.57 |
| 4 | 1978.4223 | 329.0526 | A | 1978.4141 | 9.272 | 715798 | 100 | 4.14 |
| 3 | 1649.3697 | 305.0413 | C | 1649.3622 | 7.894 | 182946 | 100 | 4.55 |
| 2 | 1344.3284 | 305.0412 | C | 1344.3226 | 7.901 | 369846 | 100 | 4.31 |
| 1 | 1039.2872 | 345.0475 | G | 1039.2824 | 8.816 | 397016 | 100 | 4.62 |

TABLE S22
LC/MS analysis of 3′biotin-labeled RNA #8, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7151.1160 | 329.0525 | A | 7151.0928 | 12.317 | 87850496 | 100 | 3.24 |
| 19 | 6822.0635 | 305.0413 | C | 6822.0257 | 12.046 | 836640 | 100 | 5.54 |
| 18 | 6517.0222 | 329.0526 | A | 6516.9906 | 12.178 | 1896420 | 100 | 4.85 |
| 17 | 6187.9696 | 305.0412 | C | 6187.9538 | 11.973 | 51293 | 97.5 | 2.55 |
| 16 | 5882.9284 | 306.0253 | U | 5882.8973 | 11.690 | 2436562 | 100 | 5.29 |
| 15 | 5576.9031 | 345.0475 | G | 5576.8745 | 11.763 | 2954102 | 100 | 5.13 |
| 14 | 5231.8556 | 329.0525 | A | 5231.8307 | 11.780 | 1503563 | 100 | 4.76 |
| 13 | 4902.8031 | 305.0413 | C | 4902.7787 | 11.376 | 1728477 | 100 | 4.98 |
| 12 | 4597.7618 | 329.0525 | A | 4597.7384 | 11.440 | 3528610 | 100 | 5.09 |
| 11 | 4268.7093 | 306.0253 | U | 4268.6887 | 10.855 | 1721343 | 100 | 4.83 |
| 10 | 3962.6840 | 345.0474 | G | 3962.6651 | 10.805 | 2353609 | 100 | 4.77 |
| 9 | 3617.6366 | 345.0475 | G | 3617.6199 | 10.832 | 1863580 | 100 | 4.62 |
| 8 | 3272.5891 | 329.0525 | A | 3272.5764 | 10.649 | 230927 | 100 | 3.88 |
| 7 | 2943.5366 | 305.0413 | C | 2943.5235 | 10.040 | 1417986 | 100 | 4.45 |
| 6 | 2638.4953 | 306.0253 | U | 2638.4844 | 9.867 | 2035557 | 100 | 4.13 |
| 5 | 2332.4700 | 345.0474 | G | 2332.4613 | 9.878 | 2467172 | 100 | 3.73 |
| 4 | 1987.4226 | 329.0525 | A | 1987.4147 | 10.359 | 2158002 | 100 | 3.97 |
| 3 | 1658.3701 | 329.0526 | A | 1658.3625 | 9.410 | 70871 | 100 | 4.58 |
| 2 | 1329.3175 | 306.0253 | U | 1329.3130 | 8.639 | 37300 | 100 | 3.39 |
| 1 | 1023.2922 | 329.0525 | A | 1023.2883 | 10.597 | 1731424 | 100 | 3.81 |

TABLE S23
LC/MS analysis of 3′biotin-labeled RNA #9, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7193.0524 | 345.0474 | G | 7193.0274 | 11.807 | 55442324 | 100 | 3.48 |
| 19 | 6848.0050 | 305.0413 | C | 6847.9696 | 11.629 | 605416 | 99.2 | 5.17 |
| 18 | 6542.9637 | 345.0474 | G | 6542.9287 | 11.612 | 1153241 | 100 | 5.35 |
| 17 | 6197.9163 | 345.0475 | G | 6197.8868 | 11.627 | 1710951 | 100 | 4.76 |
| 16 | 5852.8688 | 329.0525 | A | 5852.8355 | 11.750 | 1889983 | 100 | 5.69 |
| 15 | 5523.8163 | 306.0253 | U | 5523.7916 | 11.276 | 1055262 | 100 | 4.47 |
| 14 | 5217.7910 | 306.0253 | U | 5217.7646 | 11.181 | 2644440 | 100 | 5.06 |
| 13 | 4911.7657 | 306.0253 | U | 4911.7562 | 11.195 | 2901850 | 100 | 1.93 |
| 12 | 4605.7404 | 329.0525 | A | 4605.7117 | 10.639 | 54327 | 100 | 6.23 |
| 11 | 4276.6879 | 345.0474 | G | 4276.6684 | 12.237 | 1747514 | 100 | 4.56 |
| 10 | 3931.6405 | 305.0413 | C | 3931.6227 | 10.370 | 1744474 | 100 | 4.53 |
| 9 | 3626.5992 | 306.0253 | U | 3626.5834 | 10.080 | 2028011 | 100 | 4.36 |
| 8 | 3320.5739 | 305.0413 | C | 3320.5607 | 9.905 | 1675877 | 100 | 3.98 |
| 7 | 3015.5326 | 329.0525 | A | 3015.5209 | 10.128 | 2926950 | 100 | 3.88 |
| 6 | 2686.4801 | 345.0475 | G | 2686.4700 | 9.355 | 1768713 | 100 | 3.76 |
| 5 | 2341.4326 | 306.0253 | U | 2341.4237 | 8.811 | 1667926 | 100 | 3.80 |
| 4 | 2035.4073 | 306.0253 | U | 2035.3998 | 8.419 | 1823836 | 100 | 3.68 |
| 3 | 1729.3820 | 345.0474 | G | 1729.3764 | 8.342 | 1574679 | 100 | 3.24 |
| 2 | 1384.3346 | 345.0474 | G | 1384.3290 | 8.383 | 897954 | 100 | 4.05 |
| 1 | 1039.2872 | 345.0475 | G | 1039.2827 | 8.811 | 725527 | 100 | 4.33 |

TABLE S24
LC/MS analysis of 3′biotin-labeled RNA #10, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7088.0826 | 305.0413 | C | 7088.0613 | 11.883 | 83257784 | 100 | 3.01 |
| 19 | 6783.0413 | 329.0525 | A | 6783.0061 | 11.975 | 2374953 | 100 | 5.19 |
| 18 | 6453.9888 | 305.0413 | C | 6453.9468 | 11.681 | 1388931 | 100 | 6.51 |
| 17 | 6148.9475 | 329.0525 | A | 6148.9140 | 11.935 | 1819504 | 100 | 5.45 |
| 16 | 5819.8950 | 329.0525 | A | 5819.8674 | 11.838 | 1894041 | 100 | 4.74 |
| 15 | 5490.8425 | 329.0525 | A | 5490.8152 | 11.586 | 2817326 | 100 | 4.97 |
| 14 | 5161.7900 | 306.0253 | U | 5161.7648 | 11.083 | 2176473 | 100 | 4.88 |
| 13 | 4855.7647 | 306.0253 | U | 4855.7413 | 10.915 | 3237261 | 100 | 4.82 |
| 12 | 4549.7394 | 305.0413 | C | 4549.7141 | 10.730 | 2960106 | 100 | 5.56 |
| 11 | 4244.6981 | 345.0475 | G | 4244.6787 | 10.741 | 3118826 | 100 | 4.57 |
| 10 | 3899.6506 | 345.0474 | G | 3899.6401 | 10.625 | 2939016 | 100 | 2.69 |
| 9 | 3554.6032 | 306.0253 | U | 3554.5892 | 10.396 | 2535213 | 100 | 3.94 |
| 8 | 3248.5779 | 306.0253 | U | 3248.5652 | 9.955 | 114648 | 100 | 3.91 |
| 7 | 2942.5526 | 305.0413 | C | 2942.5417 | 9.980 | 2735803 | 100 | 3.70 |
| 6 | 2637.5113 | 306.0253 | U | 2637.5011 | 9.974 | 2936338 | 100 | 3.87 |
| 5 | 2331.4860 | 329.0525 | A | 2331.4784 | 9.985 | 2893702 | 100 | 3.26 |
| 4 | 2002.4335 | 305.0413 | C | 2002.4268 | 10.028 | 41002 | 93.6 | 3.35 |
| 3 | 1697.3922 | 329.0525 | A | 1697.3866 | 9.786 | 3139447 | 100 | 3.30 |
| 2 | 1368.3397 | 329.0525 | A | 1368.3343 | 9.551 | 1604226 | 100 | 3.95 |
| 1 | 1039.2872 | 345.0475 | G | 1039.2827 | 8.817 | 981598 | 100 | 4.33 |

TABLE S25
LC/MS analysis of 3′biotin-labeled RNA #11, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 21 | 7522.1050 | 345.0475 | G | 7522.0727 | 9.677 | 10294695 | 100 | 4.29 |
| 20 | 7177.0575 | 305.0413 | C | 7176.9932 | 9.555 | 20884 | 73.8 | 8.96 |
| 19 | 6872.0162 | 345.0474 | G | 6871.9425 | 9.511 | 77150 | 80 | 10.72 |
| 18 | 6526.9688 | 345.0474 | G | 6526.9038 | 9.494 | 106806 | 97 | 9.96 |
| 17 | 6181.9214 | 329.0526 | A | 6181.8843 | 9.576 | 410798 | 92.9 | 6.00 |
| 16 | 5852.8688 | 306.0253 | U | 5852.8373 | 9.197 | 106865 | 89.5 | 5.38 |
| 15 | 5546.8435 | 306.0253 | U | 5546.8112 | 9.073 | 412694 | 98.4 | 5.82 |
| 14 | 5240.8182 | 306.0253 | U | 5240.7832 | 8.977 | 298557 | 99.2 | 6.68 |
| 13 | 4934.7929 | 329.0525 | A | 4934.7688 | 9.053 | 289020 | 100 | 4.88 |
| 12 | 4605.7404 | 345.0474 | G | 4605.7156 | 8.603 | 217621 | 100 | 5.38 |
| 11 | 4260.6930 | 305.0413 | C | 4260.6680 | 8.429 | 242965 | 100 | 5.87 |
| 10 | 3955.6517 | 306.0253 | U | 3955.6316 | 8.244 | 345563 | 100 | 5.08 |
| 9 | 3649.6264 | 305.0413 | C | 3649.6065 | 8.024 | 410186 | 100 | 5.45 |
| 8 | 3344.5851 | 329.0525 | A | 3344.5699 | 8.115 | 552137 | 100 | 4.54 |
| 7 | 3015.5326 | 345.0474 | G | 3015.5116 | 7.460 | 373904 | 100 | 6.96 |
| 6 | 2670.4852 | 306.0253 | U | 2670.4729 | 7.068 | 332059 | 100 | 4.61 |
| 5 | 2364.4599 | 306.0253 | U | 2364.4490 | 6.658 | 358553 | 100 | 4.61 |
| 4 | 2058.4346 | 345.0475 | G | 2058.4263 | 6.345 | 313197 | 100 | 4.03 |
| 3 | 1713.3871 | 345.0474 | G | 1713.3799 | 6.069 | 197197 | 100 | 4.20 |
| 2 | 1368.3397 | 345.0475 | G | 1368.3325 | 6.214 | 146520 | 100 | 5.26 |
| 1 | 1023.2922 | 329.0525 | A | 1023.2863 | 7.215 | 150107 | 100 | 5.77 |

TABLE S26
LC/MS analysis of 5′sulfo-Cy3-labeled RNA #1, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 19 | 6859.0606 | 249.0862 | A | 6859.0293 | 13.278 | 29162746 | 100 | 4.56 |
| 18 | 6609.9744 | 329.0525 | A | 6610.9354 | 13.116 | 69218 | 73.2 | −145.39 |
| 17 | 6280.9219 | 329.0525 | A | 6280.8859 | 13.138 | 299442 | 88.9 | 5.73 |
| 16 | 5951.8694 | 329.0525 | A | 5951.8447 | 13.077 | 150172 | 80 | 4.15 |
| 15 | 5622.8169 | 305.0413 | C | 5622.7793 | 12.955 | 260581 | 80 | 6.69 |
| 14 | 5317.7756 | 305.0413 | C | 5318.7555 | 13.012 | 19172 | 68 | −184.27 |
| 13 | 5012.7343 | 329.0525 | A | 5012.7020 | 12.996 | 242326 | 94 | 6.44 |
| 12 | 4683.6818 | 345.0475 | G | 4683.6584 | 12.898 | 685126 | 86.8 | 5.00 |
| 11 | 4338.6343 | 306.0253 | U | 4338.6115 | 12.875 | 640041 | 100 | 5.26 |
| 10 | 4032.6090 | 305.0413 | C | 4032.5867 | 12.881 | 306999 | 96.3 | 5.53 |
| 9 | 3727.5677 | 329.0525 | A | 3727.5518 | 12.967 | 86034 | 81 | 4.27 |
| 8 | 3398.5152 | 345.0474 | G | 3398.5004 | 12.795 | 1050778 | 99.2 | 4.35 |
| 7 | 3053.4678 | 306.0253 | U | 3053.4581 | 12.691 | 33763 | 83.2 | 3.18 |
| 6 | 2747.4425 | 305.0413 | C | 2747.4301 | 12.803 | 244796 | 80 | 4.51 |
| 5 | 2442.4012 | 306.0253 | U | 2442.3910 | 12.791 | 1013984 | 100 | 4.18 |
| 4 | 2136.3759 | 329.0525 | A | 2136.3676 | 12.769 | 184183 | 87 | 3.89 |
| 3 | 1807.3234 | 305.0413 | C | 1807.3165 | 12.770 | 1840549 | 100 | 3.82 |
| 2 | 1502.2821 | 345.0474 | G | 1502.2765 | 12.794 | 965042 | 100 | 3.73 |
| 1 | 1157.2347 | 305.0413 | C | 1157.2297 | 13.642 | 913331 | 100 | 4.32 |

TABLE S27
LC/MS analysis of 5′sulfo-Cy3-labeled RNA #2, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7157.0696 | 225.0750 | C | 7157.0369 | 13.199 | 18994320 | 100 | 4.57 |
| 19 | 6931.9946 | 345.0474 | G | 6931.0867 | 13.257 | 166222 | 80 | 130.97 |
| 18 | 6586.9472 | 305.0413 | C | 6586.9163 | 13.247 | 373645 | 80 | 4.69 |
| 17 | 6281.9059 | 329.0525 | A | 6281.8475 | 13.280 | 216088 | 80 | 9.30 |
| 16 | 5952.8534 | 306.0253 | U | 5952.8248 | 13.205 | 931428 | 100 | 4.80 |
| 15 | 5646.8281 | 305.0413 | C | 5646.8065 | 13.222 | 627329 | 98.1 | 3.83 |
| 14 | 5341.7868 | 306.0253 | U | 5341.7543 | 13.240 | 209385 | 80.5 | 6.08 |
| 13 | 5035.7615 | 345.0474 | G | 5035.7355 | 13.256 | 355370 | 80 | 5.16 |
| 12 | 4690.7141 | 329.0526 | A | 4690.6877 | 13.288 | 293771 | 97.3 | 5.63 |
| 11 | 4361.6615 | 305.0412 | C | 4361.6393 | 13.183 | 624454 | 100 | 5.09 |
| 10 | 4056.6203 | 306.0253 | U | 4056.5940 | 13.154 | 22971 | 78.6 | 6.48 |
| 9 | 3750.5950 | 345.0475 | G | 3750.5764 | 13.218 | 392405 | 96.7 | 4.96 |
| 8 | 3405.5475 | 329.0525 | A | 3405.5311 | 13.266 | 376785 | 96.2 | 4.82 |
| 7 | 3076.4950 | 305.0413 | C | 3076.4812 | 13.144 | 764082 | 95.3 | 4.49 |
| 6 | 2771.4537 | 305.0413 | C | 2771.4461 | 13.182 | 576176 | 100 | 2.74 |
| 5 | 2466.4124 | 305.0413 | C | 2466.4028 | 13.212 | 258560 | 100 | 3.89 |
| 4 | 2161.3711 | 345.0474 | G | 2161.3628 | 13.277 | 548722 | 80 | 3.84 |
| 3 | 1816.3237 | 329.0525 | A | 1816.3169 | 13.474 | 783483 | 83.6 | 3.74 |
| 2 | 1487.2712 | 306.0253 | U | 1487.2656 | 13.532 | 1797103 | 100 | 3.77 |
| 1 | 1181.2459 | 329.0525 | A | 1181.2408 | 13.861 | 824092 | 100 | 4.32 |

TABLE S28
LC/MS analysis of 5′sulfo-Cy3-labeled RNA #3, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7166.0699 | 265.0811 | G | 7166.0350 | 13.464 | 15752947 | 100 | 4.87 |
| 19 | 6900.9888 | 329.0525 | A | 6900.9425 | 13.428 | 275366 | 81.7 | 6.71 |
| 18 | 6571.9363 | 345.0474 | G | 6571.8939 | 13.356 | 132733 | 74.1 | 6.45 |
| 17 | 6226.8889 | 306.0253 | U | 6226.8593 | 13.354 | 180552 | 77.6 | 4.75 |
| 16 | 5920.8636 | 305.0413 | C | 5920.8086 | 13.403 | 212136 | 80 | 9.29 |
| 15 | 5615.8223 | 329.0525 | A | 5615.7902 | 13.426 | 260478 | 80 | 5.72 |
| 14 | 5286.7698 | 306.0253 | U | 5286.7436 | 13.348 | 876722 | 90.9 | 4.96 |
| 13 | 4980.7445 | 306.0253 | U | 4980.7386 | 13.371 | 654236 | 100 | 1.18 |
| 12 | 4674.7192 | 329.0526 | A | 4674.6993 | 13.424 | 542251 | 80 | 4.26 |
| 11 | 4345.6666 | 305.0413 | C | 4345.6466 | 13.329 | 814417 | 100 | 4.60 |
| 10 | 4040.6253 | 305.0412 | C | 4040.6052 | 13.361 | 520867 | 97.8 | 4.97 |
| 9 | 3735.5841 | 329.0526 | A | 3735.5739 | 13.419 | 42982 | 59.3 | 2.73 |
| 8 | 3406.5315 | 306.0253 | U | 3406.5151 | 13.318 | 770893 | 95.9 | 4.81 |
| 7 | 3100.5062 | 306.0253 | U | 3100.4930 | 13.340 | 491826 | 100 | 4.26 |
| 6 | 2794.4809 | 345.0474 | G | 2794.4683 | 13.348 | 371969 | 93.7 | 4.51 |
| 5 | 2449.4335 | 305.0413 | C | 2449.4233 | 13.398 | 303466 | 80 | 4.16 |
| 4 | 2144.3922 | 305.0413 | C | 2144.3829 | 13.436 | 419905 | 86.4 | 4.34 |
| 3 | 1839.3509 | 329.0525 | A | 1839.3429 | 13.365 | 179583 | 85.7 | 4.35 |
| 2 | 1510.2984 | 329.0525 | A | 1510.2924 | 13.403 | 288879 | 79.7 | 3.97 |
| 1 | 1181.2459 | 329.0525 | A | 1181.2410 | 13.860 | 707398 | 100 | 4.15 |

TABLE S29
LC/MS analysis of 5′sulfo-Cy3-labeled RNA #4, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7063.0304 | 225.0749 | C | 7063.0072 | 13.390 | 11257376 | 87 | 3.28 |
| 19 | 6837.9555 | 306.0253 | U | 6837.9201 | 13.469 | 300823 | 85.7 | 5.18 |
| 18 | 6531.9302 | 305.0413 | C | 6531.9373 | 13.584 | 30910 | 80 | −1.09 |
| 17 | 6226.8889 | 306.0253 | U | 6226.8376 | 13.627 | 26579 | 60 | 8.24 |
| 16 | 5920.8636 | 305.0413 | C | 5920.8443 | 13.631 | 50737 | 74.8 | 3.26 |
| 15 | 5615.8223 | 329.0525 | A | 5615.7920 | 13.671 | 42482 | 62.8 | 5.40 |
| 14 | 5286.7698 | 305.0413 | C | 5286.7615 | 13.594 | 843779 | 83.1 | 1.57 |
| 13 | 4981.7285 | 329.0525 | A | 4981.6999 | 13.636 | 151248 | 97 | 5.74 |
| 12 | 4652.6760 | 306.0254 | U | 4652.6511 | 13.391 | 1191688 | 87 | 5.35 |
| 11 | 4346.6506 | 305.0412 | C | 4346.6371 | 13.403 | 130923 | 69.6 | 3.11 |
| 10 | 4041.6094 | 305.0413 | C | 4041.5867 | 13.571 | 376672 | 93.2 | 5.62 |
| 9 | 3736.5681 | 306.0253 | U | 3736.5502 | 13.588 | 60297 | 97.3 | 4.79 |
| 8 | 3430.5428 | 329.0525 | A | 3430.5239 | 13.454 | 45199 | 69.4 | 5.51 |
| 7 | 3101.4903 | 305.0413 | C | 3101.4769 | 13.301 | 778223 | 99.5 | 4.32 |
| 6 | 2796.4490 | 329.0526 | A | 2796.4353 | 13.695 | 35158 | 77.6 | 4.90 |
| 5 | 2467.3964 | 329.0525 | A | 2467.3855 | 13.818 | 108974 | 88.2 | 4.42 |
| 4 | 2138.3439 | 329.0525 | A | 2138.3355 | 13.161 | 82910 | 88.3 | 3.93 |
| 3 | 1809.2914 | 306.0253 | U | 1809.2846 | 13.153 | 2193742 | 87 | 3.76 |
| 2 | 1503.2661 | 345.0474 | G | 1503.2598 | 13.708 | 159632 | 100 | 4.19 |
| 1 | 1158.2187 | 306.0253 | U | 1158.2131 | 14.188 | 1574057 | 100 | 4.84 |

TABLE S30
LC/MS analysis of 5′sulfo-Cy3-labeled RNA #5, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7151.0590 | 249.0862 | A | 7151.0256 | 13.949 | 43379424 | 100 | 4.67 |
| 19 | 6901.9728 | 345.0474 | G | 6901.9226 | 13.835 | 322593 | 80 | 7.27 |
| 18 | 6556.9254 | 329.0525 | A | 6556.8915 | 13.831 | 396493 | 80 | 5.17 |
| 17 | 6227.8729 | 329.0525 | A | 6227.8645 | 13.834 | 76663 | 79.1 | 1.35 |
| 16 | 5898.8204 | 305.0413 | C | 5898.7973 | 13.640 | 212566 | 69.6 | 3.92 |
| 15 | 5593.7791 | 306.0253 | U | 5593.7475 | 13.745 | 664008 | 80 | 5.65 |
| 14 | 5287.7538 | 305.0413 | C | 5287.7257 | 13.749 | 1458044 | 100 | 5.31 |
| 13 | 4982.7125 | 329.0525 | A | 4982.6855 | 13.742 | 174109 | 80 | 5.42 |
| 12 | 4653.6600 | 305.0413 | C | 4653.6362 | 13.697 | 2006854 | 100 | 5.11 |
| 11 | 4348.6187 | 329.0525 | A | 4348.5989 | 13.647 | 73164 | 72.2 | 4.55 |
| 10 | 4019.5662 | 306.0253 | U | 4019.5481 | 13.535 | 920778 | 83.9 | 4.50 |
| 9 | 3713.5409 | 306.0253 | U | 3713.5244 | 13.672 | 120519 | 100 | 4.44 |
| 8 | 3407.5156 | 345.0475 | G | 3407.4991 | 13.681 | 168659 | 71.6 | 4.84 |
| 7 | 3062.4681 | 329.0525 | A | 3062.4534 | 13.557 | 126015 | 87 | 4.80 |
| 6 | 2733.4156 | 329.0525 | A | 2733.4027 | 13.604 | 327314 | 98.1 | 4.72 |
| 5 | 2404.3631 | 305.0413 | C | 2404.3530 | 13.326 | 2389580 | 87 | 4.20 |
| 4 | 2099.3218 | 306.0253 | U | 2099.3075 | 13.169 | 17723 | 91 | 6.81 |
| 3 | 1793.2965 | 306.0253 | U | 1793.2897 | 13.865 | 217546 | 98.6 | 3.79 |
| 2 | 1487.2712 | 329.0525 | A | 1487.2657 | 13.677 | 2638249 | 100 | 3.70 |
| 1 | 1158.2187 | 306.0253 | U | 1158.2134 | 14.192 | 2172695 | 100 | 4.58 |

TABLE S31
LC/MS analysis of 5′sulfo-Cy3-labeled RNA #6, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7032.9709 | 226.0590 | U | 7032.9380 | 12.938 | 24081534 | 100 | 4.68 |
| 19 | 6806.9119 | 329.0525 | A | 6806.8565 | 12.954 | 497938 | 100 | 8.14 |
| 18 | 6477.8594 | 306.0253 | U | 6477.8296 | 12.875 | 1123636 | 100 | 4.60 |
| 17 | 6171.8341 | 306.0253 | U | 6171.7982 | 12.889 | 797659 | 100 | 5.82 |
| 16 | 5865.8088 | 306.0253 | U | 5865.8484 | 12.899 | 1419968 | 80 | −6.75 |
| 15 | 5559.7835 | 305.0413 | C | 5559.7761 | 12.919 | 249723 | 80 | 1.33 |
| 14 | 5254.7422 | 305.0413 | C | 5254.7165 | 12.944 | 1499456 | 100 | 4.89 |
| 13 | 4949.7009 | 305.0413 | C | 4949.6783 | 12.982 | 147053 | 79.4 | 4.57 |
| 12 | 4644.6596 | 305.0413 | C | 4644.6354 | 13.000 | 1219024 | 100 | 5.21 |
| 11 | 4339.6183 | 306.0253 | U | 4339.6137 | 13.021 | 1246558 | 100 | 1.06 |
| 10 | 4033.5930 | 306.0253 | U | 4033.5760 | 13.029 | 1640640 | 100 | 4.21 |
| 9 | 3727.5677 | 305.0412 | C | 3727.5530 | 13.039 | 726317 | 96.4 | 3.94 |
| 8 | 3422.5265 | 306.0253 | U | 3422.5122 | 13.068 | 1753331 | 100 | 4.18 |
| 7 | 3116.5012 | 329.0526 | A | 3116.4876 | 13.113 | 1248491 | 100 | 4.36 |
| 6 | 2787.4486 | 305.0413 | C | 2787.4373 | 12.970 | 2163746 | 94.6 | 4.05 |
| 5 | 2482.4073 | 329.0525 | A | 2482.3979 | 13.002 | 695135 | 100 | 3.79 |
| 4 | 2153.3548 | 306.0253 | U | 2153.3470 | 12.883 | 2141185 | 100 | 3.62 |
| 3 | 1847.3295 | 345.0474 | G | 1847.3226 | 12.935 | 1062104 | 100 | 3.74 |
| 2 | 1502.2821 | 305.0413 | C | 1502.2770 | 13.140 | 2211201 | 100 | 3.39 |
| 1 | 1197.2408 | 345.0474 | G | 1197.2362 | 13.279 | 1324255 | 97.5 | 3.84 |

TABLE S32
LC/MS analysis of 5′sulfo-Cy3-labeled RNA #7, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7188.0754 | 265.0811 | G | 7188.0577 | 13.257 | 198372 | 69.6 | 2.46 |
| 19 | 6922.9943 | 305.0413 | C | 6922.9600 | 13.374 | 1169126 | 80 | 4.95 |
| 18 | 6617.9530 | 305.0413 | C | 6617.9032 | 13.372 | 360353 | 74.5 | 7.52 |
| 17 | 6312.9117 | 329.0525 | A | 6312.8754 | 13.386 | 707713 | 80 | 5.75 |
| 16 | 5983.8592 | 345.0474 | G | 5983.8242 | 13.343 | 112885 | 76.8 | 5.85 |
| 15 | 5638.8118 | 345.0475 | G | 5638.7821 | 13.268 | 961515 | 80 | 5.27 |
| 14 | 5293.7643 | 305.0412 | C | 5293.7168 | 13.185 | 35206 | 74.5 | 8.97 |
| 13 | 4988.7231 | 305.0413 | C | 4988.7064 | 13.196 | 35019 | 80 | 3.35 |
| 12 | 4683.6818 | 305.0413 | C | 4683.6599 | 13.355 | 148461 | 76.4 | 4.68 |
| 11 | 4378.6405 | 306.0253 | U | 4378.6236 | 13.355 | 51270 | 72.7 | 3.86 |
| 10 | 4072.6152 | 329.0525 | A | 4072.5932 | 13.368 | 444401 | 80 | 5.40 |
| 9 | 3743.5627 | 345.0475 | G | 3743.5471 | 13.261 | 227634 | 87 | 4.17 |
| 8 | 3398.5152 | 306.0253 | U | 3398.4868 | 13.177 | 17855 | 60.3 | 8.36 |
| 7 | 3092.4899 | 345.0474 | G | 3092.4781 | 13.125 | 168338 | 100 | 3.82 |
| 6 | 2747.4425 | 306.0253 | U | 2747.4316 | 13.187 | 1180398 | 80 | 3.97 |
| 5 | 2441.4172 | 329.0525 | A | 2441.4095 | 13.120 | 42956 | 69.5 | 3.15 |
| 4 | 2112.3647 | 305.0413 | C | 2112.3571 | 13.052 | 1527354 | 100 | 3.60 |
| 3 | 1807.3234 | 305.0413 | C | 1807.3165 | 13.069 | 1451369 | 100 | 3.82 |
| 2 | 1502.2821 | 345.0474 | G | 1502.2772 | 13.207 | 113774 | 68.3 | 3.26 |
| 1 | 1157.2347 | 305.0413 | C | 1157.2301 | 13.961 | 766397 | 100 | 3.97 |

TABLE S33
LC/MS analysis of 5′sulfo-Cy3-labeled RNA #8, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7229.1033 | 249.0862 | A | 7229.0695 | 14.003 | 20040654 | 100 | 4.68 |
| 19 | 6980.0171 | 306.0253 | U | 6979.9807 | 13.902 | 342469 | 93 | 5.21 |
| 18 | 6673.9918 | 329.0525 | A | 6673.9395 | 13.923 | 96589 | 80 | 7.84 |
| 17 | 6344.9393 | 329.0525 | A | 6344.8887 | 13.883 | 446012 | 100 | 7.97 |
| 16 | 6015.8868 | 345.0475 | G | 6015.8539 | 13.811 | 789692 | 100 | 5.47 |
| 15 | 5670.8393 | 306.0253 | U | 5670.8112 | 13.810 | 791636 | 100 | 4.96 |
| 14 | 5364.8140 | 305.0413 | C | 5364.7851 | 13.819 | 362044 | 80 | 5.39 |
| 13 | 5059.7727 | 329.0525 | A | 5059.7461 | 13.868 | 339561 | 93.8 | 5.26 |
| 12 | 4730.7202 | 345.0474 | G | 4730.6953 | 13.791 | 747218 | 100 | 5.26 |
| 11 | 4385.6728 | 345.0475 | G | 4385.6481 | 13.785 | 214489 | 94.3 | 5.63 |
| 10 | 4040.6253 | 306.0253 | U | 4040.6034 | 13.783 | 610851 | 95.5 | 5.42 |
| 9 | 3734.6000 | 329.0525 | A | 3734.5797 | 13.829 | 119982 | 80 | 5.44 |
| 8 | 3405.5475 | 305.0413 | C | 3405.5304 | 13.722 | 821756 | 100 | 5.02 |
| 7 | 3100.5062 | 329.0525 | A | 3100.4915 | 13.818 | 232602 | 96.9 | 4.74 |
| 6 | 2771.4537 | 345.0474 | G | 2771.4408 | 13.716 | 597795 | 98 | 4.65 |
| 5 | 2426.4063 | 306.0253 | U | 2426.3956 | 13.699 | 984832 | 100 | 4.41 |
| 4 | 2120.3810 | 305.0413 | C | 2120.3722 | 13.781 | 756259 | 100 | 4.15 |
| 3 | 1815.3397 | 329.0525 | A | 1815.3334 | 13.920 | 202800 | 69.6 | 3.47 |
| 2 | 1486.2872 | 305.0413 | C | 1486.2813 | 13.960 | 1082970 | 100 | 3.97 |
| 1 | 1181.2459 | 329.0525 | A | 1181.2406 | 14.173 | 183863 | 100 | 4.49 |

TABLE S34
LC/MS analysis of 5′sulfo-Cy3-labeled RNA #9, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7271.0397 | 265.0811 | G | 7271.0150 | 13.447 | 60392476 | 100 | 3.40 |
| 19 | 7005.9586 | 345.0474 | G | 7005.9069 | 13.414 | 1544260 | 80 | 7.38 |
| 18 | 6660.9112 | 345.0474 | G | 6660.8417 | 13.401 | 115234 | 80 | 10.43 |
| 17 | 6315.8638 | 306.0253 | U | 6315.8150 | 13.385 | 1000227 | 98.5 | 7.73 |
| 16 | 6009.8385 | 306.0253 | U | 6009.8074 | 13.404 | 2545935 | 100 | 5.17 |
| 15 | 5703.8132 | 345.0475 | G | 5703.7940 | 13.412 | 2410664 | 100 | 3.37 |
| 14 | 5358.7657 | 329.0525 | A | 5358.7424 | 13.432 | 2729923 | 100 | 4.35 |
| 13 | 5029.7132 | 305.0413 | C | 5029.6903 | 13.335 | 4588952 | 100 | 4.55 |
| 12 | 4724.6719 | 306.0253 | U | 4724.6524 | 13.354 | 3608892 | 100 | 4.13 |
| 11 | 4418.6466 | 305.0413 | C | 4418.6275 | 13.370 | 2676034 | 100 | 4.32 |
| 10 | 4113.6053 | 345.0474 | G | 4113.5871 | 13.360 | 2671523 | 100 | 4.42 |
| 9 | 3768.5579 | 329.0525 | A | 3768.5431 | 13.376 | 2388710 | 100 | 3.93 |
| 8 | 3439.5054 | 306.0253 | U | 3439.4913 | 13.239 | 5653201 | 100 | 4.10 |
| 7 | 3133.4801 | 306.0253 | U | 3133.4702 | 13.243 | 5267381 | 100 | 3.16 |
| 6 | 2827.4548 | 306.0253 | U | 2827.4447 | 13.246 | 5120720 | 100 | 3.57 |
| 5 | 2521.4295 | 329.0525 | A | 2521.4201 | 13.262 | 2676447 | 100 | 3.73 |
| 4 | 2192.3770 | 345.0475 | G | 2192.3695 | 13.169 | 4164446 | 100 | 3.42 |
| 3 | 1847.3295 | 345.0474 | G | 1847.3232 | 13.196 | 3096228 | 100 | 3.41 |
| 2 | 1502.2821 | 305.0413 | C | 1502.7018 | 13.404 | 105885 | 100 | −279.37 |
| 1 | 1197.2408 | 345.0474 | G | 1197.2365 | 13.559 | 2206497 | 100 | 3.59 |

TABLE S35
LC/MS analysis of 5′sulfo-Cy3-labeled RNA #10, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7166.0699 | 265.0811 | G | 7166.0414 | 13.739 | 69199560 | 100 | 3.98 |
| 19 | 6900.9888 | 329.0525 | A | 6900.9627 | 13.715 | 919150 | 84.4 | 3.78 |
| 18 | 6571.9363 | 329.0525 | A | 6571.8812 | 13.658 | 1047891 | 80 | 8.38 |
| 17 | 6242.8838 | 305.0413 | C | 6242.8539 | 13.597 | 1775042 | 86.7 | 4.79 |
| 16 | 5937.8425 | 329.0525 | A | 5937.8328 | 13.633 | 1623713 | 100 | 1.63 |
| 15 | 5608.7900 | 306.0253 | U | 5608.7668 | 13.540 | 3247803 | 83.7 | 4.14 |
| 14 | 5302.7647 | 305.0413 | C | 5302.7398 | 13.580 | 2133663 | 80 | 4.70 |
| 13 | 4997.7234 | 306.0253 | U | 4997.6996 | 13.526 | 95112 | 98.9 | 4.76 |
| 12 | 4691.6981 | 306.0253 | U | 4691.6768 | 13.611 | 2450965 | 100 | 4.54 |
| 11 | 4385.6728 | 345.0475 | G | 4385.6522 | 13.605 | 1676478 | 95.3 | 4.70 |
| 10 | 4040.6253 | 345.0474 | G | 4040.6081 | 13.541 | 840397 | 87 | 4.26 |
| 9 | 3695.5779 | 305.0413 | C | 3695.5619 | 13.592 | 2706026 | 100 | 4.33 |
| 8 | 3390.5366 | 306.0253 | U | 3390.5202 | 13.605 | 2811611 | 80 | 4.84 |
| 7 | 3084.5113 | 306.0253 | U | 3084.5013 | 13.627 | 2928850 | 80 | 3.24 |
| 6 | 2778.4860 | 329.0525 | A | 2778.4757 | 13.655 | 1989313 | 80 | 3.71 |
| 5 | 2449.4335 | 329.0525 | A | 2449.4242 | 13.610 | 2067865 | 95.4 | 3.80 |
| 4 | 2120.3810 | 329.0525 | A | 2120.3722 | 13.528 | 1449232 | 80 | 4.15 |
| 3 | 1791.3285 | 305.0413 | C | 1791.3228 | 13.583 | 1030070 | 87 | 3.18 |
| 2 | 1486.2872 | 329.0525 | A | 1486.2816 | 13.482 | 1294548 | 80.7 | 3.77 |
| 1 | 1157.2347 | 305.0413 | C | 1157.2299 | 14.136 | 1103912 | 87 | 4.15 |

TABLE S36
LC/MS analysis of 5′sulfo-Cy3-labeled RNA #11, showing its mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 21 | 7600.0923 | 249.0862 | A | 7600.0602 | 13.263 | 21803014 | 100 | 4.22 |
| 20 | 7351.0061 | 345.0475 | G | 7350.9572 | 13.133 | 208063 | 80 | 6.65 |
| 19 | 7005.9586 | 345.0474 | G | 7007.9075 | 13.126 | 20219 | 85 | −278.18 |
| 18 | 6660.9112 | 345.0474 | G | 6660.8639 | 13.117 | 272418 | 80 | 7.10 |
| 17 | 6315.8638 | 306.0253 | U | 6315.8230 | 13.105 | 213624 | 80 | 6.46 |
| 16 | 6009.8385 | 306.0253 | U | 6009.7925 | 13.119 | 469394 | 80 | 7.65 |
| 15 | 5703.8132 | 345.0475 | G | 5703.7807 | 13.125 | 307370 | 100 | 5.70 |
| 14 | 5358.7657 | 329.0525 | A | 5358.7543 | 13.143 | 797008 | 98.5 | 2.13 |
| 13 | 5029.7132 | 305.0413 | C | 5029.6880 | 13.054 | 1304776 | 100 | 5.01 |
| 12 | 4724.6719 | 306.0253 | U | 4724.6479 | 13.077 | 822977 | 100 | 5.08 |
| 11 | 4418.6466 | 305.0413 | C | 4418.6277 | 13.098 | 935202 | 100 | 4.28 |
| 10 | 4113.6053 | 345.0474 | G | 4113.5865 | 13.091 | 823731 | 100 | 4.57 |
| 9 | 3768.5579 | 329.0525 | A | 3768.5416 | 13.108 | 903026 | 100 | 4.33 |
| 8 | 3439.5054 | 306.0253 | U | 3439.4924 | 12.970 | 1748702 | 100 | 3.78 |
| 7 | 3133.4801 | 306.0253 | U | 3133.4698 | 12.975 | 1760722 | 100 | 3.29 |
| 6 | 2827.4548 | 306.0253 | U | 2827.4439 | 12.980 | 1762939 | 100 | 3.86 |
| 5 | 2521.4295 | 329.0525 | A | 2521.4208 | 12.994 | 454731 | 100 | 3.45 |
| 4 | 2192.3770 | 345.0475 | G | 2192.3692 | 12.904 | 1509385 | 100 | 3.56 |
| 3 | 1847.3295 | 345.0474 | G | 1847.3231 | 12.929 | 1224721 | 100 | 3.46 |
| 2 | 1502.2821 | 305.0413 | C | 1502.2770 | 13.128 | 1429495 | 100 | 3.39 |
| 1 | 1197.2408 | 345.0474 | G | 1197.2365 | 13.271 | 832362 | 100 | 3.59 |

TABLE S37
LC/MS analysis of 3′biotin-labeled RNA #12, showing
its ψ-CMC-converted mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7485.3767 | 329.0525 | A | 7485.3910 | 15.919 | 4301601 | 100 | −1.91 |
| 19 | 7156.3242 | 329.0525 | A | 7157.3265 | 15.917 | 39821 | 100 | −140.06 |
| 18 | 6827.2717 | 329.0525 | A | 6827.3170 | 15.917 | 56899 | 79.7 | −6.64 |
| 17 | 6498.2192 | 305.0413 | C | 6498.2258 | 15.896 | 30478 | 80 | −1.02 |
| 16 | 6193.1779 | 305.0413 | C | 6193.1808 | 15.928 | 47149 | 78.9 | −0.47 |
| 15 | 5888.1366 | 345.0475 | G | 5888.0997 | 15.924 | 84778 | 80 | 6.27 |
| 14 | 5543.0891 | 306.0253 | U | 5543.1180 | 15.924 | 132659 | 80 | −5.21 |
| 13 | 5237.0638 | 557.2251 | Converted | 5237.0573 | 15.949 | 40639 | 80 | 1.24 |
| | | | ψ | | | | | |
| 12 | 4679.8387 | 329.0525 | A | 4679.8399 | 14.581 | 2275437 | 87.5 | −0.26 |
| 11 | 4350.7862 | 305.0413 | C | 4350.7877 | 14.104 | 356588 | 83.8 | −0.34 |
| 10 | 4045.7449 | 305.0413 | C | 4045.7446 | 14.070 | 158059 | 91 | 0.07 |
| 9 | 3740.7036 | 329.0525 | A | 3740.7018 | 14.437 | 797927 | 100 | 0.48 |
| 8 | 3411.6511 | 306.0253 | U | 3411.6501 | 13.988 | 281415 | 95.7 | 0.29 |
| 7 | 3105.6258 | 306.0253 | U | 3105.6155 | 13.593 | 11367 | 93 | 3.32 |
| 6 | 2799.6005 | 329.0525 | A | 2799.5971 | 14.370 | 30839 | 100 | 1.21 |
| 5 | 2470.5480 | 319.0570 | m⁵C | 2470.5463 | 14.271 | 3260900 | 100 | 0.69 |
| 4 | 2151.4910 | 306.0253 | U | 2151.4861 | 13.612 | 379983 | 78.8 | 2.28 |
| 3 | 1845.4657 | 345.0474 | G | 1845.4639 | 14.006 | 2888007 | 90.9 | 0.98 |
| 2 | 1500.4183 | 329.0525 | A | 1500.4155 | 14.916 | 8695 | 65.8 | 1.87 |
| 1 | 1171.3658 | 345.0475 | G | 1171.3639 | 15.168 | 1223164 | 80 | 1.62 |

TABLE S38
LC/MS analysis of 3′biotin-labeled RNA #12, showing its ψ-unconverted mass ladder components.

| Theoretical | | | | Extracted data file after LC/MS analysis | | | | |
|---|---|---|---|---|---|---|---|---|
| | | Base | | MFE | | | Quality | Error |
| Fragments | Theoretical mass | mass | Base | mass | RT | Volume | Score | ppm |
| 20 | 7234.1769 | 329.0525 | A | 7234.1945 | 14.789 | 341038 | 100 | −2.43 |
| 19 | 6905.1244 | 329.0525 | A | 6905.1351 | 14.831 | 41837 | 80 | −1.55 |
| 18 | 6576.0719 | 329.0525 | A | 6576.0763 | 14.646 | 147954 | 80.9 | −0.67 |
| 17 | 6247.0194 | 305.0413 | C | 6247.0289 | 14.269 | 194023 | 98.6 | −1.52 |
| 16 | 5941.9781 | 305.0413 | C | 5941.9839 | 14.269 | 208740 | 100 | −0.98 |
| 15 | 5636.9368 | 345.0475 | G | 5636.9382 | 14.273 | 30200 | 80.4 | −0.25 |
| 14 | 5291.8893 | 306.0253 | U | 5291.8772 | 14.187 | 12930 | 90.7 | 2.29 |
| 13 | 4985.8640 | 306.0253 | Unconverted | 4985.8436 | 14.236 | 19666 | 90.2 | 4.09 |
| | | | ψ | | | | | |
| 12 | 4679.8387 | 329.0525 | A | 4679.8399 | 14.581 | 2275437 | 87.5 | −0.26 |
| 11 | 4350.7862 | 305.0413 | C | 4350.7877 | 14.104 | 356588 | 83.8 | −0.34 |
| 10 | 4045.7449 | 305.0413 | C | 4045.7446 | 14.070 | 158059 | 91 | 0.07 |
| 9 | 3740.7036 | 329.0525 | A | 3740.7018 | 14.437 | 797927 | 100 | 0.48 |
| 8 | 3411.6511 | 306.0253 | U | 3411.6501 | 13.988 | 281415 | 95.7 | 0.29 |
| 7 | 3105.6258 | 306.0253 | U | 3105.6155 | 13.593 | 11367 | 93 | 3.32 |
| 6 | 2799.6005 | 329.0525 | A | 2799.5971 | 14.370 | 30839 | 100 | 1.21 |
| 5 | 2470.5480 | 319.0570 | m⁵C | 2470.5463 | 14.271 | 3260900 | 100 | 0.69 |
| 4 | 2151.4910 | 306.0253 | U | 2151.4861 | 13.612 | 379983 | 78.8 | 2.28 |
| 3 | 1845.4657 | 345.0474 | G | 1845.4639 | 14.006 | 2888007 | 90.9 | 0.98 |
| 2 | 1500.4183 | 329.0525 | A | 1500.4155 | 14.916 | 8695 | 65.8 | 1.87 |
| 1 | 1171.3658 | 345.0475 | G | 1171.3639 | 15.168 | 1223164 | 80 | 1.62 |

The data was processed as the following:
For FIG. 16B: Maximum plotting window RT set to 20: ax.set_ylim(min_time, 15); Maximum Mass<7000. Take the top 500 by Volume (above and including 3486)
FIG. 16C: plotting window RT set to between 5.5 and 12: ax.set_ylim(5.5,12) Maximum Mass<7500
Take the top 500 by volume (above and including 1219)

### FIG. 17B: Maximum Mass<7000

Take the top 1000 by volume (above and including 33693)

### FIG. 19A: Maximum Mass<8000

Take the top 500 by volume (above and including 241698)

### FIG. 19B: Maximum Mass<8000

Take the top 1000 by volume because the CMC-labeling efficiency was somewhat low (above and including 63110)

### FIG. S2: Maximum Mass<8000

Take the top 300 by volume (above and including 121230)
The second step is to analyze the LC/MS data and automatically recognize the RNA sequences.
A modified version of the algorithm from [JACS 2015] was used.
A modification was first made to the default.cfg file:

### Before

| | |
|---|---|
| −WALK_STEPS_MIN_DRAFT, 10 | # draft minimum number |

of steps per walk (before orientation determination)

| | |
|---|---|
| −WALK_STEPS_MIN_FINAL, 14 | # final minimum number of |
| | steps per walk |

| | |
|---|---|
| −WALK_PPM, 5.0 | # maximum allowable mass |

error (ppm) during walk traversal

### After

| | |
|---|---|
| +WALK_STEPS_MIN_DRAFT, 5 | # draft minimum number |

of steps per walk (before orientation determination)

| | |
|---|---|
| +WALK_STEPS_MIN_FINAL, 8 | # final minimum number of |
| | steps per walk |

| | |
|---|---|
| +WALK_PPM, 10.0 | # maximum allowable mass |

error (ppm) during walk traversal

1) the requirement of a strictly monotonically increasing or decreasing sequence plot was deleted

### Commented Out:

#if len(nextpos) and nextposcall:
#if not tdir and not bidirectional:

# get the RT direction of the first step (up or down)

and use this for the remainder of the walk

```
  #tdir = int(np.sign(nextpos[‘RT’] − pos[−1][‘RT’]))
  # FOR TESTING: calculate tdir as slope
# tdir = (nextpos[‘RT’] − pos[−1][‘RT’])/(nextpos[‘Mass'] −
pos[−1][‘Mass'])
```

2) a mass filtering step was disabled:

# apply the selected filters
#if PARAMS_[‘FILTER_MIN_MASS’] is not None:
#cdb.filter(mass=(PARAMS_[‘FILTER_MIN_MASS’],
startingpos[‘Mass']))

3) For FIG. 16C and later, the following regions of the code were commented out for ease of plotting to remove the labels.

| | |
|---|---|
| ## | for c in cpd: |
| ## | for ft, i in zip(trials, range(len(trials))): |

| | |
|---|---|
| ## | if (c in [f[‘Cpd’] for f in ft]) and (c not in top_c): |
| ## | p = [x for x in compounds if x[‘Cpd’] == c][0] |
| ## | if orientations[i]: |

| | |
|---|---|
| ## | top_m.append(p[‘Mass']) |
| ## | top_v.append(p[‘Vol’]) |
| ## | top_t.append(p[‘RT’]) |
| ## | top_c.append(p[‘Cpd’]) |

| | |
|---|---|
| ## | else: |

| | |
|---|---|
| ## | bottom_m.append(p[‘Mass']) |
| ## | bottom_v.append(p[‘Vol’]) |
| ## | bottom_t.append(p[‘RT’]) |
| ## | bottom_c.append(p[‘Cpd’]) |

if len(bottom_m):
p4 = plt.scatter(bottom_m, bottom_t, c=bottom_v, s=msize, edgecolor=‘k’,

linewidth=1, marker=‘o’, alpha=alphahigh, cmap=cmap, norm=norm, zorder=3)

cbar = fig.colorbar(p4)

```
  if len(top_m):
  p3 = plt.scatter(top_m, top_t, c=top_v, s=msize, edgecolor=‘k’, linewidth=1,
```

marker=‘s',

| alpha=alphahigh, cmap=cmap, norm=norm, zorder=3) | |
|---|---|
| if ‘cbar’ in locals( ): | |
| cbar.vmin = np.min(np.hstack((top_v, bottom_v))) | |
| cbar.vmax = np.max(np.hstack((top_v, bottom_v))) | |
| else: | |
| cbar = fig.colorbar(p3) | |
| ## | #plot trial walks |
| ## | for i in range(len(trials)): |

| | |
|---|---|
| ## | if orientations[i]: |
| ## | plt.plot([f[‘Mass'] for f in trials[i]], [f[‘RT’] for f in trials[i]], ‘k−’, |

| | |
|---|---|
| ## | alpha=alphahigh, linewidth=1, zorder=2) |

| | |
|---|---|
| ## | else: |
| ## | plt.plot([f[‘Mass'] for f in trials[i]], [f[‘RT’] for f in trials[i]], ‘k−’, |

| | |
|---|---|
| ## | alpha=alphahigh, linewidth=1, zorder=2) |

| ## | |
|---|---|
| ## | if plot_labels: |

| | |
|---|---|
| ## | ann1 = [ ] |
| ## | ann0 = [ ] |
| ## | for trial, orientation in zip(trials, orientations): |
| ## | for i in range(len(trial) − 1): |

| | |
|---|---|
| ## | if orientation: |
| ## | a = {‘text’: baselist.findnamebyid(trial[i + 1][‘Call’]), |

| | |
|---|---|
| ## | ‘xy’: (trial[i][‘Mass'], trial[i][‘RT’]), |
| ## | ‘xytext’: (trial[i][‘Mass'] / 2 + trial[i + 1][‘Mass'] / 2 + |

| annotation_offset[0], | |
|---|---|
| ## | trial[i][‘RT’] + annotation_offset[1]), ‘color’: trial[i + |

| 1][‘WalkScore’]} | |
|---|---|
| ## | if a not in ann1: |

| | |
|---|---|
| ## | a = dodgetext(a,ann1,−1) |
| ## | if a is not None: |

| | |
|---|---|
| ## | ann1.append(a) |

| | |
|---|---|
| ## | else: |
| ## | a = {‘text’: baselist.findnamebyid(trial[i + 1][‘Call’]), |

| | |
|---|---|
| ## | ‘xy’: (trial[i][‘Mass'], trial[i][‘RT’]), |
| ## | ‘xytext’: (trial[i][‘Mass'] / 2 + trial[i + 1][‘Mass'] / 2 − |

| annotation_offset[0], | |
|---|---|
| ## | trial[i][‘RT’] − annotation_offset[1]), ‘color’: trial[i + |

| 1][‘WalkScore’]} | |
|---|---|
| ## | if a not in ann0: |

| | |
|---|---|
| ## | a = dodgetext(a,ann0,1) |
| ## | if a is not None: |

| | |
|---|---|
| ## | ann0.append(a) |

| | |
|---|---|
| ## | ann = [ ] |
| ## | for a in chain(ann0, ann1): |
| ## | ann.append(ax.annotate(a[‘text’], a[‘xy’], |

| horizontalalignment=‘center’, verticalalignment=‘center’, | |
|---|---|
| ## | textcoords=‘data’, xytext=a[‘xytext’], |
| ## | arrowprops=dict(arrowstyle=“—”, color=‘#999999’, |

| | |
|---|---|
| ## | alpha=alphalow, |

| ## | connectionstyle=“angle,angleA=0,angleB=90,rad= |
|---|---|
| 0”), | |
| ## | color=‘k’)) |

| ## | |
|---|---|
| ## | elif len(trials): |
| ## | p1 = plt.scatter(m, t, c=v, s=msize, linewidth=0, alpha=alphahigh, |

| cmap=cmap, norm=norm, | |
|---|---|
| ## | zorder=1) |

| | |
|---|---|
| ## | # plot trial walks |
| ## | for i in range(len(trials)): |

| | |
|---|---|
| ## | plt.plot([f[‘Mass'] for f in trials[i]], [f[‘RT’] for f in trials[i]], ‘k−’, |

| | |
|---|---|
| ## | alpha=alphahigh, linewidth=1, zorder=2) |

| | |
|---|---|
| ## | else: |
| ## | p1 = plt.scatter(m, t, c=v, s=msize, linewidth=0, alpha=alphahigh, |

```
  cmap=cmap, norm=norm, zorder=1)
  ##
```

if ‘cbar’ not in locals( ):
cbar = fig.colorbar(p1)

| ## | |
|---|---|
| ## | if plot_midline and len(midline): |
| ## | p2 = plt.plot(midline[:, 0], midline[:, 1], ‘k−.’, zorder=1) |

Additional changes for specific figures were done according to the following:
For FIG. 16B: Maximum plotting window RT set to 20: ax.set_ylim(min_time, 20)

### Maximum Mass<7000

Take the top 500 by Volume (above and including 3486)
The plotting direction was also flipped (changes in bold):

```
    if plot_labels:
  ann1 = [ ]
ann0 = [ ]
  for trial, orientation in zip(trials, orientations):
  for i in range(len(trial) − 1):
if orientation:
```

a = {‘text’: baselist.findnamebyid(trial[i + 1][‘Call’]),

```
  ‘xy’: (trial[i][‘Mass'], trial[i][‘RT’]),
  ‘xytext’: (trial[i][‘Mass'] / 2 + trial[i + 1][‘Mass'] / 2 +
annotation_offset[0],
```

```
trial[i][‘RT’] + annotation_offset[1]), ‘color’: trial[i + 1][‘WalkScore’]}
  if a not in ann1:
    a = dodgetext(a,ann1,−1)
if a is not None:
ann1.append(a)
    else:
a = {‘text’: baselist.findnamebyid(trial[i + 1][‘Call’]),
‘xy’: (trial[i][‘Mass'], trial[i][‘RT’]),
  ‘xytext’: (trial[i][‘Mass'] / 2 + trial[i + 1][‘Mass'] /
  2 − annotation_offset[0],
trial[i][‘RT’] − annotation_offset[1]), ‘color’: trial[i +
1][‘WalkScore’]}
if a not in ann0:
    a = dodgetext(a,ann0,1)
if a is not None:
ann0.append(a)
```

All patents, patent applications and references cited throughout the specification are expressly incorporated by reference.

### REFERENCES

1 Warren, E. N., Elms, P. J., Parker, C. E. & Borchers, C. H. Development of a protein chip: a MS-based method for quantitation of protein expression and modification levels using an immunoaffinity approach. Anal Chem 76, 4082-4092, doi:10.1021/ac049880g (2004).
2 Lu, L. et al. Association of large noncoding RNA HOTAIR expression and its downstream intergenic CpG island methylation with survival in breast cancer. Breast Cancer Res Treat 136, 875-883, doi:10.1007/s10549-012-2314-z (2012).
3 Jiang, J., Aduri, R., Chow, C. S. & SantaLucia, J., Jr. Structure modulation of helix 69 from Escherichia coli 23S ribosomal RNA by pseudouridylations. Nucleic Acids Res 42, 3971-3981, doi:10.1093/nar/gkt1329 (2014).
4 Wang, H. L. & Lai, W. Y. Profiling DNA and RNA Modifications Using Advanced LC-MS/MS Technologies. Lc Gc N Am 35, 521-522 (2017).
5 Thuring, K., Schmid, K., Keller, P. & Helm, M. LC-MS Analysis of Methylated RNA. Methods Mol Biol 1562, 3-18, doi:10.1007/978-1-4939-6807-7_1 (2017).
6 Bjorkbom, A. et al. Bidirectional Direct Sequencing of Noncanonical RNA by Two-Dimensional Analysis of Mass Chromatograms. J Am Chem Soc 137, 14430-14438, doi:10.1021/jacs.5b09438 (2015).
7 Balatti, V., Pekarsky, Y. & Croce, C. M. Role of the tRNA-Derived Small RNAs in Cancer: New Potential Biomarkers and Target for Therapy. Adv Cancer Res 135, 173-187, doi:10.1016/bs.acr.2017.06.007 (2017).
8 Torres, A. G., Batlle, E. & Ribas de Pouplana, L. Role of tRNA modifications in human diseases. Trends Mol Med 20, 306-314, doi:10.1016/j.molmed.2014.01.008 (2014).
9 Hori, H. Methylated nucleosides in tRNA and tRNA methyltransferases. Front Genet 5, 144, doi:10.3389/fgene.2014.00144 (2014).
10 Blanco, S. et al. Aberrant methylation of tRNAs links cellular stress to neuro-developmental disorders. EMBO J 33, 2020-2039, doi:10.15252/embj.201489282 (2014).
11 Zheng, G. et al. Efficient and quantitative high-throughput tRNA sequencing. Nat Methods 12, 835-837, doi:10.1038/nmeth.3478 (2015).
12. Cantara et al. The RNA modification database, RNAMDB:2011 update. Nucleic Acids Research, 2011, Vol. 39, Database issue D195-D201
13. Thomas B & Akoulitchev A V (2006) Mass spectrometry of RNA. Trends Biochem Sci 31(3)173-181].
14. Bjorkbom, A. et al. Bidirectional Direct Sequencing of Noncanonical RNA by Two-Dimensional Analysis of Mass Chromatograms. J Am Chem Soc 137, 14430-14438 (2015).
15. Cole, K., Truong, V., Barone, D. & McGall, G. Direct labeling of RNA with multiple biotins allows sensitive expression profiling of acute leukemia class predictor genes. Nucleic Acids Res 32, e86 (2004).
16. Adachi, H., De Zoysa, M. D. & Yu, Y. T. Post-transcriptional pseudouridylation in mRNA as well as in some major types of noncoding RNAs. Biochim Biophys Acta Gene Regul Mech (2018).
17. Harcourt, E. M., Kietrys, A. M. & Kool, E. T. Chemical and structural effects of base modifications in messenger RNA. Nature 541, 339-346 (2017).
18. Bakin, A. & Ofengand, J. Four newly located pseudouridylate residues in Escherichia coli 23S ribosomal RNA are all at the peptidyltransferase center: analysis by the application of a new sequencing technique. Biochemistry 32, 9754-9762 (1993).
19. Roundtree, I. A., et al. Cell 2017 169:1187-1200;
20. Meyer et al., Annu Rev. Cell Dev Biol 2017 33:319-342)
21. Zhang et al., Proc. Natl. Acd. Sci USA 2013 44:17732-17737)
22. Zhang et al. J/Am. Chem Soc. 2013 135:924-32.

1. An RNA sequencing method, for determining the primary RNA sequence and the presence/identification/location of RNA modifications, comprising the steps of: (i) labeling of the 5′ and/or 3′ end of the RNA; (ii) random degradation of the RNA; (iii) optionally, physical separation of resultant RNA fragments based on 5′ and 3′ end labeling; (iv) separation and detection of the resultant RNA fragment properties; and (v) data analysis resulting in sequence/modification identification.

2. The method of claim 1, wherein the step (iv) separation of resultant RNA fragments is achieved by high performance liquid chromatography or by capillary electrophoresis.

3-4. (canceled)

5. The method of claim 1 wherein the step (iv) detection of resultant RNA fragment properties is achieved through mass spectrometry.

6. The RNA sequencing method of claim 1, wherein the affinity labeling of the 5′ and/or 3′ end of the RNA molecule is selected from the group consisting of (i) a hydrophobic label like a biotin or a fluorescent dye such as CY3 or CY5; (ii) a thiol group; (iii) any biotinylated pCp; (iv) a DNA adapter; and (v) a poly(A) oligonucleotide.

7-10. (canceled)

11. The RNA sequencing method of claim 1, wherein the chemical degradation of the RNA is performed by chemical degradation.

12. (canceled)

13. The RNA sequencing method of claim 1, wherein the degradation of the RNA is performed by enzymatic degradation.

14. (canceled)

15. The RNA sequencing method of claim 1, wherein the chemical degradation is performed before the affinity labeling of the 5′ and 3′ end of the RNA molecule.

16. The RNA sequencing method of claim 1, wherein the chemical degradation is performed after the affinity labeling of the 5′ and 3′ end of the RNA molecule.

17. The RNA sequencing method of claim 1, wherein the RNA sample comprises an RNA selected from the group consisting of the following: (i) a purified RNA sample of limited diversity; (ii) a mixture of RNAs; (iii) a therapeutic RNA molecule; and (iv) an analog of an RNA molecule.

18-19. (canceled)

20. The RNA sequencing method of claim 1, wherein the RNA nucleotide sequence is determined by correlation of MS data output with the mass of know and/or unknown ribonucleosides.

21. The RNA sequencing method of claim 1, wherein the presence of modified ribonucleosides is determined by correlation of MS data output with the mass of known and/or unknown modified ribonucleosides.

22. An RNA sequencing method comprising the steps of: (i) labeling of the 5′ and/or 3′ end of the RNA with a moiety that increases the hydrophobicity of the RNA fragments thereby increasing the retention time of degraded RNA fragments; (ii) random degradation of the RNA; (iii) separation and detection of the resultant RNA fragment properties; and (iv) data analysis resulting in sequence/modification identification.

23. The method of claim 22, wherein the step (iii) separation of resultant RNA fragments is achieved by high performance liquid chromatography or by capillary electrophoresis.

24. The method of claim 22, wherein the high performance liquid chromatography is reverse phase high performance liquid chromatography.

25. (canceled)

26. The method of claim 22 wherein the step (iii) detection of resultant RNA fragment properties is achieved through mass spectrometry.

27. The method of claim 22 wherein (i) the 3′ end of the RNA is labeled with a biotin moiety and the 5′ end of the RNA is labeled with a hydrophobic Cy3 tag or (ii) the 5′ end of the RNA is labeled with a biotin moiety and the 3′ end of the RNA is labeled with a hydrophobic Cy3 tag.

28. A DNA sequencing method comprising the steps of: (i) affinity labeling of the 5′ and/or 3′ end of the DNA; (ii) random degradation of the DNA into mass ladders; (iii) optionally, physical separation of resultant DNA fragments based on an affinity interaction; (iv) measurement of resultant DNA fragments using reverse-phase high performance liquid chromatography (HPLC) or capillary electrophoresis (CE) or other separation methods coupled with mass spectrometry; and (v) MS data analysis resulting in sequence/modification identification.

29. The DNA sequencing method of claim 28, wherein the affinity labeling of the 5′ and/or 3′ end of the DNA molecule is with a biotin label.

30. The DNA sequencing method of claim 28, wherein the degradation of the DNA is performed by enzymatic degradation.

31. (canceled)

32. The DNA sequencing method of claim 1, wherein data analysis is a two (2) dimensional analysis that relies on mass and retention times; or (ii) is performed based on the unique properties of RNA fragments resultant from the RNA sequence.

33. (canceled)

34. The DNA sequencing method of claim 32, wherein the unique properties of RNA fragments are electronic or optical signature signals.

35. The RNA sequencing method of claim 1, wherein RNA containing modified nucleoside pseudouridine (ψ) is treated with CMC, where CMC preferentially reacts with ψ over uridine (U), resulting in a formation of a CMC-ψ adduct and wherein the adduct results in mass and RT shifts over non-CMC-converted ψ including U in the 2-D mass-RT plot.

36. An RNA sequencing method wherein the RNA is ψ-containing, comprising the steps of: (i) treatment of RNA to be sequenced with CMC; (ii) affinity labeling of the 5′ and 3′ end of the RNA; (iii) random degradation of the RNA; (iv) optionally, physical separation of resultant RNA fragments based on an affinity interaction; (v) measurement of resultant RNA fragments using reverse-phase high performance liquid chromatography (HPLC) or capillary electrophoresis (CE) or other separation methods coupled with mass spectrometry; and (v) MS data analysis resulting in sequence/modification identification.

37. (canceled)

38. The RNA sequencing method of claim 1, wherein the RNA sequence including modified nucleobases is determined from a mixture containing both modified and non-modified RNA and wherein the relative percentage of modified nucleobases versus non-modified nucleobases can be quantified.

39-40. (canceled)

41. The RNA sequencing method of claim 17, wherein the analog of the RNA molecule is N3′-P5′-linked phosphoramidate DNA or RNA.
